SEQUENCE LISTING (22 sequences)

SEQ ID NO: 1 - DNA, 660 bp, synthetic construct
  source 1..660: mol_type = other DNA
ggatccgccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggagtctagataagaattc

SEQ ID NO: 2 - DNA, 510 bp, Homo sapiens
  source 1..510: mol_type = genomic DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtg

SEQ ID NO: 3 - DNA, 257 bp, synthetic construct
  source 1..257: mol_type = other DNA
ccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagccc

SEQ ID NO: 4 - DNA, 81 bp, Homo sapiens
  source 1..81: mol_type = genomic DNA
ttttgggtgctggtggtggttggtggagtcctggcttgctatagcttgctagtaacagtggcctttattattttctgggtg

SEQ ID NO: 5 - DNA, 462 bp, Homo sapiens
  source 1..462: mol_type = genomic DNA
aggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 6 - DNA, 1341 bp, synthetic construct
  source 1..1341: mol_type = other DNA
ggatccgccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgctcgaggctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagaattc

SEQ ID NO: 7 - DNA, 201 bp, Homo sapiens
  source 1..201: mol_type = genomic DNA
gagacgggactccagaaagaccatgccctctgggatcacactgcccagaatctccttcggatgggcctggcctttctagtcctggtggctctagtgtggttcctggttgaagactggctcagcaggaagaggactagagagcgagccagcagagcttccacttgggaaggcaggagaaggctgaacacacagactctttga

SEQ ID NO: 8 - DNA, 741 bp, synthetic construct
  source 1..741: mol_type = other DNA
ggatccgccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgctcgaggagacgggactccagaaagaccatgccctctgggatcacactgcccagaatctccttcggatgggcctggcctttctagtcctggtggctctagtgtggttcctggttgaagactggctcagcaggaagaggactagagagcgagccagcagagcttccacttgggaaggcaggagaaggctgaacacacagactctttgatctagagaattc

SEQ ID NO: 9 - DNA, 228 bp, Homo sapiens
  source 1..228: mol_type = genomic DNA
cagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggctga

SEQ ID NO: 10 - DNA, 279 bp, Homo sapiens
  source 1..279: mol_type = genomic DNA
ctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 11 - DNA, 633 bp, Homo sapiens
  source 1..633: mol_type = genomic DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggag

SEQ ID NO: 12 - DNA, 54 bp, Homo sapiens
  source 1..54: mol_type = genomic DNA
agccccgcccaagaagatggcaaagtctacatcaacatgccaggcaggggctga

SEQ ID NO: 13 - DNA, 126 bp, Homo sapiens
  source 1..126: mol_type = genomic DNA
ggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 14 - DNA, 768 bp, synthetic construct
  source 1..768: mol_type = other DNA
ggatccgccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgctcgagcagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggctgatctagagaattc

SEQ ID NO: 15 - DNA, 819 bp, synthetic construct
  source 1..819: mol_type = other DNA
ggatccgccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgctcgagctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgatctagagaattc

SEQ ID NO: 16 - DNA, 711 bp, synthetic construct
  source 1..711: mol_type = other DNA
ggatccgccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggagtctagaagccccgcccaagaagatggcaaagtctacatcaacatgccaggcaggggctgagaattc

SEQ ID NO: 17 - DNA, 783 bp, synthetic construct
  source 1..783: mol_type = other DNA
ggatccgccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggagtctagaggggctgcggaggcagcgacccggaaacagcgtatcactgagaccgagtcgccttatcaggagctccagggtcagaggtcggatgtctacagcgacctcaacacacagaggccgtattacaaatgagaattc

SEQ ID NO: 18 - DNA, 7829 bp, synthetic construct
  source 1..7829: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagtcgaggtcgacggtatcgataagctcgcttcacgagattccagcaggtcgagggacctaataacttcgtatagcatacattatacgaagttatattaagggttccaagcttaagcggccggccgctgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgcccggatcccagtgtggtggtacgggaattcctgcaggcctcgacgagggccggcgcgccgcggccgctacgtaaattccgccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccggccgccagcacagtggtcgaaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 19 - DNA, 7208 bp, synthetic construct
  source 1..7208: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagtcgaggtcgacggtatcgataagctcgcttcacgagattccagcaggtcgagggacctaataacttcgtatagcatacattatacgaagttatattaagggttccaagcttaagcggccgctgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgcccggatccgccaccatggctaagctaggaagcggagagggcagaggcagtctgctgacatgcggtgacgtggaagagaatcccggccctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 20 - DNA, 23 bp, synthetic construct
  source 1..23: mol_type = other DNA
taccgctgcatgatcagctatgg

SEQ ID NO: 21 - DNA, 23 bp, synthetic construct
  source 1..23: mol_type = other DNA
taccatactctaccacatatagg

SEQ ID NO: 22 - DNA, 810 bp, synthetic construct
  source 1..810: mol_type = other DNA
ggatccgccaccatggctaagctaggaagcggagagggcagaggcagtctgctgacatgcggtgacgtggaagagaatcccggccctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattc

(19) United States
(12) Patent Application Publication
(10) Pub. No.: US 2025/0034226 A1
(43) Pub. Date: Jan. 30, 2025

(54) CHIMERIC SWITCH RECEPTORS IN NK CELLS

(71) Applicants: Evren ALICI, Huddinge (SE); Katharina SUSEK, Stockholm (SE); VYCELLIX, INC, Tampa, FL (US)

(72) Inventors: Evren ALICI, Huddinge (SE); Katharina SUSEK, Stockholm (SE)

(73) Assignee: VYCELLIX, INC, Tampa, FL (US)

(21) Appl. No.: 18/716,800

(22) PCT Filed: Dec. 6, 2022
(86) PCT No.: PCT/US2022/052043

§ 371 Date: Jun. 5, 2024

Related U.S. Application Data
(60) Provisional application No. 63/286,205, filed on Dec. 6, 2021.

(51) Int. Cl. C07K 14/705; A61K 39/00; A61P 35/00; C07K 14/725; C12N 5/0783

(52) U.S. Cl. CPC C07K 14/70503; A61K 39/4613; A61K 39/4637; A61K 39/464429; A61P 35/00; C07K 14/7051; C07K 14/70521; C07K 14/70596; C12N 5/0646; A61K 2239/15; A61K 2239/46; C07K 2319/02

(57) ABSTRACT

Multiple myeloma (MM) is an incurable hematological cancer, in which immune checkpoint inhibition (ICI) with monoclonal antibodies (mAbs) has failed due to uncontrollable immune responses in combination therapies and lack of efficacy in monotherapies. NK cells have effector activity within the TME, under continuous ligand exposure. NK cell dysfunctionality may occur due to interaction of PD1 and its ligand PD-L1. We created NK cell specific PD1-based chimeric switch receptors (PD1-CSR) by employing signaling domains of DAP10, DAP12 and CD3 to revert NK cell inhibition and retarget ICI. PD1-CSR modified NK cells showed increased degranulation, cytokine secretion and cytotoxicity upon recognition of PDL1+ target cells.

### PRIORITY AND INCORPORATION BY REFERENCE

This application claims priority to U.S. provisional patent application 63/286,205 filed on Dec. 6, 2021, which application is incorporated by reference herein its entirety. All references cited herein are incorporated by reference in their entireties.

### FUNDING

This work was supported by VINNOVA (2019-00056) and Radiumhemmets forskningsfonder (191063) and the Castenbäcks Stiftelse för cancer research. KHS received a young investigator award from the International Myeloma Society (IMS).

### Ethics Approval and Consent to Participate

Ethical permits were granted by the Swedish Ethical Review board (Etikprövningsmyndighet) for work with patient derived PBMCs and bone marrow samples (permit numbers: 2019-04973 and 2020-02119).

### BACKGROUND

The field of cancer immunotherapy has shown breakthrough advances due to the success of immune checkpoint inhibition (ICI) and chimeric antigen receptor (CAR)-T cell therapy. As resistance towards ICI and adoptive cell therapies occurs, combination therapies are explored [1, 2]. One approach is to genetically modify effector cells to make them less prone to PD-L1/PD-L2-mediated inhibition. Currently, several registered clinical trials employ PD1 knockout (PD1-KO) or PD1 disrupted chimeric antigen receptor (CAR) T cells for various malignancies [3-5]. Although this approach has been proven successful in some tumor models, emerging data indicate that PD1-KO might also impair T cell functionality [6]. Therefore, another novel approach is the utilization of chimeric switch receptors (CSR) that link PD-L1 engagement to an activating signal.

Natural killer (NK) cells are innate lymphoid cells that recognize and kill infected, stressed or malignant cells without prior antigen exposure [7]. They exert direct cytotoxicity against target cells and enhance immune responses via cytokine and chemokine secretion [8]. NK cell activation depends on the balance of several germline-encoded inhibitory and activating receptors [9]. One of the strongest activating receptors is CD16 that binds to the constant region (Fc) of immunoglobulins and induces antibody-dependent cellular cytotoxicity (ADCC). Many activating receptors lack a signaling domain and rather depend on adaptor proteins for a functional response. The most prominent of these are the immunoreceptor tyrosine-based activating motif (ITAM)-bearing adaptor proteins CD3ζ and DAP12 as well as DAP10 which signals via a YINM motif [9-11].

In a recent clinical trial, CD19-CAR-NK cells displayed a good clinical response with seven out of eleven patients reaching a complete remission with only minimal toxicity [12]. Adoptive cell therapies, employing NK cells, are thus increasingly becoming important due to several reasons such as a beneficial risk profile [13]. However, there are still many open questions to ensure the success of NK cell-based immunotherapies in the clinical setting. For example, the concern of NK cell hypofunctionality due to immune-checkpoint receptor engagement in the tumor microenvironment (TME) has not previously been resolved. Although the role of PD1 on NK cells from healthy individuals is not fully understood, it has been shown that tumor-infiltrating NK cells often show increased PD1 expression with reduced effector cell functionality that can be reverted by PD1-PD-L1 blockade with mAb [14-18]. Therefore, there remains a need to assess the ability of PD1-based CSRs to sustain the functionality of NK cells against different PD-L1⁺ tumor targets.

### BRIEF DESCRIPTION OF THE INVENTION

Herein we demonstrate, among other things, that PD1-CSR expressing NK-92 and primary NK (pNK) cells increase degranulation, cytokine secretion, and tumor cell killing upon recognition of PD-L1.

In certain embodiments, provided herein are novel NK cell specific PD1-based chimeric switch receptors (PD1-CSR) comprising signaling domains of DAP10, DAP12 and/or CD3ζ to revert NK cell inhibition and retarget ICI. PD1-CSR modified NK cells showed increased degranulation, cytokine secretion and cytotoxicity upon recognition of PD-L1⁺ target cells. Additionally, PD1-CSR⁺ NK cells infiltrated and killed tumor spheroids. While primary NK cells (pNK), expressing native PD1, showed decreased degranulation and cytokine production against PD-L1⁺ target cells by two-fold, PD1-CSR⁺ pNK cells demonstrated increased activity upon PD-L1⁺ target cell recognition and enhanced antibody-dependent cellular cytotoxicity. PD1-CSR⁺ pNK cells from patients with MM increased degranulation and cytokine expression against autologous CD138⁺ PD-L1⁺ malignant plasma cells. Taken together, the present results demonstrate that PD1-CSR⁺ NK cells enhance and sustain potent anti-tumor activity in a PD-L1⁺ microenvironment and thus represent a promising strategy to advance adoptive NK cell-based immunotherapies towards PD-L1⁺ cancers.

In one embodiment the invention comprises an NK cell specific PD1-based chimeric switch receptor. In another embodiment the invention the chimeric switch receptor optionally contains at least one signaling domain from DAP10, DAP12, NKp46 or CD3ζ In another embodiment the chimeric switch receptor comprises amino acids 1-170 of PD1 (SEQ ID NO: 2), an 85 amino acid long hinge region (SEQ ID NO 3), amino acids 153-220 of CD28 (SEQ ID NO: 4) and amino acids 52-164 of the CD3ζ protein (SEQ ID NO: 5). In another embodiment the chimeric switch receptor comprises amino acids 1-170 of PD1 (SEQ ID NO 2) fused together with amino acids 239-304 of the NKp46 protein (SEQ ID NO 7). In yet another embodiment the chimeric switch receptor comprises amino acids 1 to 170 of PD1 (SEQ ID NO: 2) fused to full length DAP10 (AA 19-93) (SEQ ID NO: 6). In some embodiments the chimeric switch receptor comprises amino acids 1 to 170 of PD1 (SEQ ID NO: 2) fused to the full length DAP12 (AA 22-113) (SEQ ID NO: 7) protein. In another embodiment the chimeric switch receptor comprises amino acids 1 to 212 of PD1 (SEQ ID NO: 11) fused together with AA 77-93 of DAP10 (SEQ ID NO: 12). In another embodiment the chimeric switch receptor comprises amino acids 1 to 212 of PD1 (SEQ ID NO: 11) fused together with amino acids 73-113 of DAP12 (SEQ ID NO 13). In another embodiment the chimeric switch receptor comprises a signaling domain and the signaling domain is truncated, preferably wherein the signaling domain is truncated to omit the transmembrane region. In another embodiment the chimeric switch receptor comprises amino acids 52-164 of the CD3ζ protein (SEQ ID NO: 5). In another embodiment the chimeric switch receptor comprises amino acids 239-304 of the NKp46 protein (SEQ ID NO 7). In another embodiment the NK cell comprises the DAP 10 construct of SEQ ID NO 14. In another embodiment the NK cell comprises the DAP 12 construct of SEQ ID NO 15. In another embodiment the NK cell comprises the DAP 10 construct of SEQ ID NO 16. In another embodiment the NK cell comprises the DAP 12 construct of SEQ ID NO 17.

In some embodiments the invention comprises treating multiple myeloma in a patient in need thereof comprising administering an NK cell specific PD1-based chimeric switch receptor. In some embodiments the chimeric switch receptor comprises at least one signaling domain from DAP10, DAP12, NKp46 or CD3ζ. In some embodiments the chimeric switch receptor comprises amino acids 1-170 of PD1 (SEQ ID NO: 2), an 85 amino acid long hinge region (SEQ ID NO 3), amino acids 153-220 of CD28 (SEQ ID NO: 4) and amino acids 52-164 of the CD3ζ protein (SEQ ID NO: 5). In some embodiments the chimeric switch receptor comprises amino acids 1-170 of PD1 (SEQ ID NO 2) fused together with amino acids 239-304 of the NKp46 protein (SEQ ID NO 7). In some embodiments the chimeric switch receptor comprises amino acids 1 to 170 of PD1 (SEQ ID NO: 2) fused to the full length DAP10 (AA 19-93) (SEQ ID NO: 6). In some embodiments the chimeric switch receptor comprises amino acids 1 to 170 of PD1 (SEQ ID NO: 2) fused to the full length DAP12 (AA 22-113) (SEQ ID NO: 7) protein. In some embodiments the chimeric switch receptor comprises amino acids 1 to 212 of PD1 (SEQ ID NO: 11) fused together with amino acids 77-93 of DAP10 (SEQ ID NO: 12). In some embodiments the chimeric switch receptor comprises amino acids 1 to 212 of PD1 (SEQ ID NO: 11) fused together with amino acids 73-113 of DAP12 (SEQ ID NO 13). In some embodiments the signaling domain is truncated to omit the transmembrane region. In some embodiments the chimeric switch receptor comprises amino acids 52-164 of the CD3ζ protein (SEQ ID NO: 5). In some embodiments the chimeric switch receptor comprises amino acids 239-304 of the NKp46 protein (SEQ ID NO 7). In other embodiments the chimeric switch receptor comprises the DAP 10 construct of SEQ ID NO 14. In some embodiments the chimeric switch receptor comprises the DAP 12 construct of SEQ ID NO 15. In some embodiments the chimeric switch receptor comprises the DAP 10 construct of SEQ ID NO 16. In other embodiments the chimeric switch receptor comprises the DAP 12 construct of SEQ ID NO 17.

Some embodiments comprise a genetic engineering construct comprising SEQ ID NO 5, SEQ ID NO 7, SEQ ID NO 14, SEQ ID NO 15, SEQ ID NO 16, or SEQ ID NO 17.

Some embodiments comprise a nucleic acid encoding an NK cell specific PD1-based chimeric switch receptor.

Some embodiments comprise a cell comprising a nucleic acid encoding an exogenous chimeric switch receptor. In some embodiments the chimeric switch receptor comprises at least one signaling domain from DAP10, DAP12, NKp46 or CD3ζ.

In some embodiments the invention comprises DNA, in others it comprises the corresponding RNA or even protein. Delivery of DNA or RNA into cells can be via any scientifically acceptable means.

### DESCRIPTION OF THE FIGURES

FIGS. 1A-F are Table and Vector Maps and graphs showing that PD1-based chimeric switch receptors are stably expressed in NK-92 cells.

FIGS. 2A-B are graphs showing that PD1-CSR⁺ NK-92 cells increase degranulation, cytokine production and killing of PD-L1⁺ target cells.

FIGS. 3A-G are images and graphs showing PD1-CSR⁺ NK-92 cells increase cytotoxicity against PD-L1⁺ tumor spheroids.

FIGS. 4A-I show graphs showing PD1-CSR⁺ pNK cells increase degranulation and cytokine production against PD-L1⁺ target cells.

FIGS. 5A-M are graphs showing that PD1-CSR⁺ pNK cells increase degranulation and cytokine production against PD-L1⁺ autologous tumor samples.

FIGS. 6A-B are flow cytometry plots showing the expression of ligands for activating NK cell receptors are not altered on target cell lines.

FIGS. 7A-D are graphs showing PD1-CSR⁺ NK-92 cells increase degranulation and cytokine production against PD-L1⁺ target cells.

FIGS. 8A-E are graphs showing that NK-92 cells stably express PD1-CSR and show no differences in PD-L1 independent degranulation.

FIGS. 9A-E are graphs showing that PD1-CSR⁺ NK-92 cells do not increase killing of PD-L1⁺ Raji cells.

FIGS. 10A-E are graphs showing that PD1-CSR⁺ NK-92 increase killing of tumor spheroids, but do not show altered proliferation.

FIG. 11 consists of graphs showing the expression of activating and inhibitory NK cell receptors on PD1-CSR⁺ pNK compared to unmodified pNK cells.

FIGS. 12A-L are graphs showing PD1-CSR⁺ pNK cells increase degranulation against PD-L1⁺ Raji cells with and without the addition of Rituximab.

FIGS. 13A-F are graphs showing that PD1-CSR-PD1^{dim} pNK cells do not increase degranulation towards PD-L1⁺ Raji cells.

FIGS. 14A-C are graphs showing PD1-CSR⁺ pNK cells do not impact target cell killing but maintain proliferation compared to PD1⁺ WT cells.

FIGS. 15A-B are graphs showing that CD38⁺CD138⁺ malignant plasma cells express ligands for activating NK cell receptors.

FIG. 16 is A map of the vector LeGO_MCS-GSG-T2A-eGFP

### DETAILED DESCRIPTION OF THE INVENTION

Multiple myeloma (MM) is an incurable hematological cancer, in which immune checkpoint inhibition (ICI) with monoclonal antibodies (mAbs) has failed due to uncontrollable immune responses in combination therapies and lack of efficacy in monotherapies. Although NK cell specific checkpoint targets such as NKG2A and KIRs are currently being evaluated in clinical trials, the clinical impact of NK cells on the PD1 cascade is less well understood compared to T cells. Furthermore, while NK cells have effector activity within the TME, under continuous ligand exposure, NK cell dysfunctionality may occur due to interaction of PD1 and its ligand PD-L1. Due to abovementioned factors, we designed novel NK cell specific PD1-based chimeric switch receptors (PD1-CSR) by employing signaling domains of DAP10, DAP12 and CD3ζ to revert NK cell inhibition and retarget ICI. PD1-CSR modified NK cells showed increased degranulation, cytokine secretion and cytotoxicity upon recognition of PD-L1⁺ target cells. Additionally, PD1-CSR⁺ NK cells infiltrated and killed tumor spheroids. While primary NK cells (pNK), expressing native PD1, showed decreased degranulation and cytokine production against PD-L1⁺ target cells by two-fold, PD1-CSR⁺ pNK cells demonstrated increased activity upon PD-L1⁺ target cell recognition and enhanced antibody-dependent cellular cytotoxicity. PD1-CSR⁺ pNK cells from patients with MM increased degranulation and cytokine expression against autologous CD138⁺PD-L1⁺ malignant plasma cells. Taken together, the present results demonstrate that PD1-CSR⁺ NK cells enhance and sustain potent anti-tumor activity in a PD-L1⁺ microenvironment and thus represent a promising strategy to advance adoptive NK cell-based immunotherapies towards PD-L1⁺ cancers.

### Methods

### Cells

All cell lines were purchased from ATCC. The B cell lymphoma cell line Raji (ATCC® CCL-86™) and the renal cell carcinoma cell line 786-0 (ATCC® CRL-1932™) were maintained in RPMI medium (Gibco), supplemented with 10% FBS (Gibco). NK-92 cells (ATCC® CRL-2407™) were maintained in SCGM (CellGenix), supplemented with 20% FBS (Gibco). Cell lines were split every 2-3 days. Interleukin-2 (R&D) was added at a final concentration of 500 U/ml to the cell culture medium of NK-92 cells.

### PBMC and Primary NK Cell Isolation and Culture

Peripheral blood mononuclear cells (PBMCs) were obtained from buffy coats.

According to institutional guidelines ethical permits were not required for healthy donors due to de-identification of donors. Ethical permits were granted for work with patient derived PBMCs and bone marrow samples (permit numbers: 2019-04973 and 2020-02119). PBMC isolation was performed with LymphoPrep™ (Fresenius Kabi) according to the manufacturer's recommendations. Isolated PBMCs were cultured in SCGM medium (CellGenix), supplemented with 5% human serum (Biowittaker). CD3 Ab (Miltenyi, clone OKT3) was added to the culture at a final concentration of 10 ng/ml on the day of isolation. Interleukin (IL)-2 (R&D) was added to the culture at a final concentration of 500 U/ml on days 1 to 4 (daily), and then five times/week. pNK cells were isolated from PBMCs by negative selection and magnetic separation according to the manufacturer's recommendations (Miltenyi, 130-092-657). pNK cells were cultured in SCGM medium (CellGenix), supplemented with 10% human serum. IL-21 (ImmunoTools) was added on the day of isolation at a concentration of 20 ng/ml. IL-2 (R&D) was added daily to the culture at a final concentration of 1,000 U/ml.

Isolation of bone marrow mononuclear cells (BM MNC)

Bone marrow aspirates were obtained from patients with MM after obtaining informed consent and according to our ethical permit (permit numbers: 2019-04973 and 2020-02119). BM MNC were isolated using Ficoll-Paque (Sigma-Aldrich) according to the manufacturer's recommendations. Isolated BM MNC were passaged in RPMI medium (Gibco), supplemented with 10% FBS (Gibco).

### Generation of Chimeric Switch Receptors

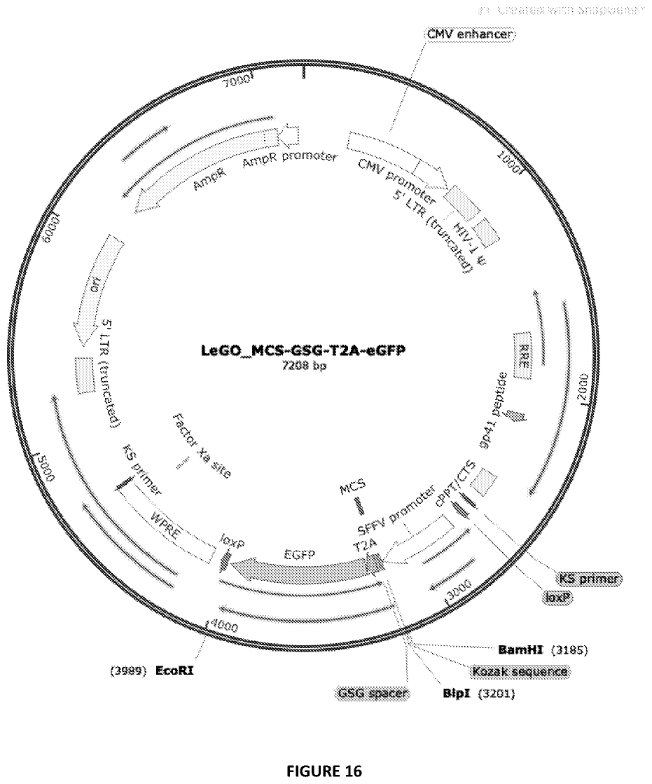

For the generation of PD1-based CSR, the canonical human cDNA sequence was used without further modification or codon optimization. A truncated version of PD1 (amino acid (AA) 1-211) (SEQ ID NO: 1), which lacks the intracellular signaling domains, was designed and is hereafter referred to as PD1_{EC-TM} CSR. The PD1-CD28-CD3ζ CSR consists of AA 1-170 of PD1 (SEQ ID NO: 2), an 85AA long hinge region, (SEQ ID NO 3) the AA sequence 153-220 of CD28 (SEQ ID NO: 4) and AA 52-164 of the CD3ζ protein (SEQ ID NO: 5). PD1-CD28-CD3ζ CSR is shown in SEQ ID NO: 6. For the PD1-NKp46 CSR, PD1 AA 1-170 (SEQ ID NO 2) was fused together with AA 239-304 of the NKp46 protein (SEQ ID NO 7). PD1-NKp46 CSR is shown as SEQ ID NO: 8). NKp46 lacks an intracellular signaling domain but can associate with ITAM-bearing CD3ζ homodimers or CD3ζ/FcεRIγ heterodimers through oppositely charged residues within the transmembrane region [19]. In the PD1_{Ec}DAP10_{TM-IC} and PD1_{Ec}DAP12_{TM-IC} constructs, PD1 protein from AA 1 to 170 (SEQ ID NO: 2) was fused to the full length DAP10 (AA 19-93) (SEQ ID NO: 9) or DAP12 (AA 22-113) (SEQ ID NO: 10) protein. In the PD1_{EC-TM}DAP10_{IC} and PD1_{EC-TM}DAP12_{IC} CSR the PD1 AA sequence 1 to 212 (SEQ ID NO: 11) was fused together with AA 77-93 of DAP10 (SEQ ID NO: 12) or AA 73-113 of DAP12 (SEQ ID NO 13). PD1_{EC-TM}DAP10_{IM-IC} is shown in SEQ ID NO: 14 PD1_{Ec}DAP12_{TM-IC} is shown in SEQ ID NO: 15. PD1_{EC-TM}DAP10_{IC} is shown in SEQ ID NO 16 and PD1_{EC-TM}DAP12_{IC} is shown in SEQ ID NO: 17. The designed constructs were cloned into the LeGoiG2 or LeGo_T2A-eGFP vector, upstream of the IRES or T2A under the control of the SFFV promoter. The LeGo_T2A-eGFP vector was designed by replacing the IRES of the LeGoG2 vector with a T2A sequence (LeGO-iG2 and LeGo-G2 were a kind gift from Dr B. Fehse) The LeGo_T2A-eGFP is shown in FIG. 16. These plasmids were used to produce VSV-G-pseudotyped lentiviral vectors.

The LeGoiG2 (Addgene plasmid 27341) vector is set forth below as SEQ ID NO: 18.
(SEQ ID NO: 18)
gtcgacggatcgggagatctcccgatcccctatggtgcactctcagtacaatctgctctgatgccgcatagttaa
gccagtatctgctccctgcttgtgtgttggaggtcgctgagtagtgcgcgagcaaaatttaagctacaacaaggcaaggctt
gaccgacaattgcatgaagaatctgcttagggttaggcgttttgcgctgcttcgcgatgtacgggccagatatacgcgttga
cattgattattgactagttattaatagtaatcaattacggggtcattagttcatagcccatatatggagttccgcgttacataa
cttacggtaaatggcccgcctggctgaccgcccaacgacccccgcccattgacgtcaataatgacgtatgttcccatagta
acgccaatagggactttccattgacgtcaatgggtggagtatttacggtaaactgcccacttggcagtacatcaagtgtatc
atatgccaagtacgccccctattgacgtcaatgacggtaaatggcccgcctggcattatgcccagtacatgaccttatggga
ctttcctacttggcagtacatctacgtattagtcatcgctattaccatggtgatgcggttttggcagtacatcaatgggcgtgg
atagcggtttgactcacggggatttccaagtctccaccccattgacgtcaatgggagtttgttttggcaccaaaatcaacgg
gactttccaaaatgtcgtaacaactccgccccattgacgcaaatgggcggtaggcgtgtacggtgggaggtctatataagc
agcgcgttttgcctgtactgggtctctctggttagaccagatctgagcctgggagctctctggctaactagggaacccactg
cttaagcctcaataaagcttgccttgagtgcttcaagtagtgtgtgcccgtctgttgtgtgactctggtaactagagatccctc
agacccttttagtcagtgtggaaaatctctagcagtggcgcccgaacagggacttgaaagcgaaagggaaaccagagga
gctctctcgacgcaggactcggcttgctgaagcgcgcacggcaagaggcgaggggcggcgactggtgagtacgccaaaa
attttgactagcggaggctagaaggagagagatgggtgcgagagcgtcagtattaagcgggggagaattagatcgcgat
gggaaaaaattcggttaaggccagggggaaagaaaaaatataaattaaaacatatagtatgggcaagcagggagctag
aacgattcgcagttaatcctggcctgttagaaacatcagaaggctgtagacaaatactgggacagctacaaccatcccttc
agacaggatcagaagaacttagatcattatataatacagtagcaaccctctattgtgtgcatcaaaggatagagataaaa
gacaccaaggaagctttagacaagatagaggaagagcaaaacaaaagtaagaccaccgcacagcaagcggccggccg
cgctgatcttcagacctggaggaggagatatgagggacaattggagaagtgaattatataaatataaagtagtaaaaatt
gaaccattaggagtagcacccaccaaggcaaagagaagagtggtgcagagagaaaaaagagcagtgggaataggagc
tttgttccttgggttcttgggagcagcaggaagcactatgggcgcagcgtcaatgacgctgacggtacaggccagacaatt
attgtctggtatagtgcagcagcagaacaatttgctgagggctattgaggcgcaacagcatctgttgcaactcacagtctgg
ggcatcaagcagctccaggcaagaatcctggctgtggaaagatacctaaaggatcaacagctcctggggatttggggttg
ctctggaaaactcatttgcaccactgctgtgccttggaatgctagttggagtaataaatctctggaacagatttggaatcac
acgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggct
gtggtatataaaattattcataatgatagtaggaggcttggtaggtttaagaatagtttttgctgtactttctatagtgaatag
agttaggcagggatattcaccattatcgtttcagacccacctcccaaccccgaggggacccgacaggcccgaaggaatag
aagaagaaggtggagagagagacagagacagatccattcgattagtgaacggatcggcactgcgtgcgccaattctgca
gacaaatggcagtattcatccacaattttaaaagaaaaggggggattggggggtacagtgcaggggaaagaatagtaga
cataatagcaacagacatacaaactaaagaattacaaaaacaaattacaaaaattcaaaattttcgggtttattacaggg
acagcagagatccagtttggttagtaccgggcccgctctagtcgaggtcgacggtatcgataagctcgcttcacgagattc
cagcaggtcgagggacctaataacttcgtatagcatacattatacgaagttatattaagggttccaagcttaagcggccgg
ccgctgaaagaccccacctgtaggtttggcaagctagctgcagtaacgccattttgcaaggcatggaaaaataccaaacc
aagaatagagaagttcagatcaagggcgggtacatgaaaatagctaacgttgggccaaacaggatatctgcggtgagca
gtttcggccccggcccggggccaagaacagatggtcaccgcagtttcggccccggcccgaggccaagaacagatggtcc
ccagatatggcccaaccctcagcagtttcttaagacccatcagatgtttccaggctcccccaaggacctgaaatgaccctgc
gccttatttgaattaaccaatcagcctgcttctcgcttctgttcgcgcgcttctgcttcccgagctctataaaagagctcacaa
cccctcactcggcgcgccagtcctccgattgactgagtcgcccggatcccagtgtggtggtacgggaattcctgcaggcctc
gacgagggccggcgcgccgcggccgctacgtaaattccgccccccccccccctctccctcccccccccctaacgttactgg
ccgaagccgcttggaataaggccggtgtgcgtttgtctatatgttattttccaccatattgccgtcttttggcaatgtgagggc
ccggaaacctggccctgtcttcttgacgagcattcctaggggtctttcccctctcgccaaaggaatgcaaggtctgttgaatg
tcgtgaaggaagcagttcctctggaagcttcttgaagacaaacaacgtctgtagcgaccctttgcaggcagcggaaccccc
cacctggcgacaggtgcctctgcggccaaaagccacgtgtataagatacacctgcaaaggcggcacaaccccagtgcca
cgttgtgagttggatagttgtggaaagagtcaaatggctctcctcaagcgtattcaacaaggggctgaaggatgcccagaa
ggtaccccattgtatgggatctgatctggggcctcggtgcacatgctttacatgtgtttagtcgaggttaaaaaaacgtctag
gccccccgaaccacggggacgtggttttcctttgaaaaacacgatgataatatggccacaaccatggtgagcaagggcga
ggagctgttcaccggggtggtgcccatcctggtcgagctggacggcgacgtaaacggccacaagttcagcgtgtccggcg
agggcgagggcgatgccacctacggcaagctgaccctgaagttcatctgcaccaccggcaagctgcccgtgccctggccc
accctcgtgaccaccctgacctacggcgtgcagtgcttcagccgctaccccgaccacatgaagcagcacgacttcttcaag
tccgccatgcccgaaggctacgtccaggagcgcaccatcttcttcaaggacgacggcaactacaagacccgcgccgaggt
gaagttcgagggcgacaccctggtgaaccgcatcgagctgaagggcatcgacttcaaggaggacggcaacatcctgggg
cacaagctggagtacaactacaacagccacaacgtctatatcatggccgacaagcagaagaacggcatcaaggtgaact
tcaagatccgccacaacatcgaggacggcagcgtgcagctcgccgaccactaccagcagaacacccccatcggcgacgg
ccccgtgctgctgcccgacaaccactacctgagcacccagtccgccctgagcaaagaccccaacgagaagcgcgatcac
atggtcctgctggagttcgtgaccgccgccgggatcactctcggcatggacgagctgtacaagtaaagcggccggccgcc
agcacagtggtcgaaattcgtcgagggacctaataacttcgtatagcatacattatacgaagttatacatgtttaagggttc
cggttccactaggtacaattcgatatcaagcttatcgataatcaacctctggattacaaaatttgtgaaagattgactggtat
tcttaactatgttgctccttttacgctatgtggatacgctgctttaatgcctttgtatcatgctattgcttcccgtatggctttcat
tttctcctccttgtataaatcctggttgctgtctctttatgaggagttgtggcccgttgtcaggcaacgtggcgtggtgtgcact
gtgtttgctgacgcaacccccactggttggggcattgccaccacctgtcagctcctttccgggactttcgctttccccctccct
attgccacggcggaactcatcgccgcctgccttgcccgctgctggacaggggctcggctgttgggcactgacaattccgtg
gtgttgtcggggaaatcatcgtcctttccttggctgctcgcctgtgttgccacctggattctgcgcgggacgtccttctgctac
gtcccttcggccctcaatccagcggaccttccttcccgcggcctgctgccggctctgcggcctcttccgcgtcttcgccttcgc
cctcagacgagtcggatctccctttgggccgcctccccgcatcgataccgtcgacctcgatcgagacctagaaaaacatgg
agcaatcacaagtagcaatacagcagctaccaatgctgattgtgcctggctagaagcacaagaggaggaggaggtgggt
tttccagtcacacctcaggtacctttaagaccaatgacttacaaggcagctgtagatcttagccactttttaaaagaaaagg
ggggactggaagggctaattcactcccaacgaagacaagatatccttgatctgtggatctaccacacacaaggctacttcc
ctgattggcagaactacacaccagggccagggatcagatatccactgacctttggatggtgctacaagctagtaccagttg
agcaagagaaggtagaagaagccaatgaaggagagaacacccgcttgttacaccctgtgagcctgcatgggatggatga
cccggagagagaagtattagagtggaggtttgacagccgcctagcatttcatcacatggcccgagagctgcatccggact
gtactgggtctctctggttagaccagatctgagcctgggagctctctggctaactagggaacccactgcttaagcctcaata
aagcttgccttgagtgcttcaagtagtgtgtgcccgtctgttgtgtgactctggtaactagagatccctcagacccttttagtc
agtgtggaaaatctctagcagcatgtgagcaaaaggccagcaaaaggccaggaaccgtaaaaaggccgcgttgctggcg
tttttccataggctccgcccccctgacgagcatcacaaaaatcgacgctcaagtcagaggtggcgaaacccgacaggact
ataaagataccaggcgtttccccctggaagctccctcgtgcgctctcctgttccgaccctgccgcttaccggatacctgtccg
cctttctcccttcgggaagcgtggcgctttctcatagctcacgctgtaggtatctcagttcggtgtaggtcgttcgctccaagc
tgggctgtgtgcacgaaccccccgttcagcccgaccgctgcgccttatccggtaactatcgtcttgagtccaacccggtaag
acacgacttatcgccactggcagcagccactggtaacaggattagcagagcgaggtatgtaggcggtgctacagagttctt
gaagtggtggcctaactacggctacactagaagaacagtatttggtatctgcgctctgctgaagccagttaccttcggaaa
aagagttggtagctcttgatccggcaaacaaaccaccgctggtagcggtggtttttttgtttgcaagcagcagattacgcgc
agaaaaaaaggatctcaagaagatcctttgatcttttctacggggtctgacgctcagtggaacgaaaactcacgttaaggg
attttggtcatgagattatcaaaaaggatcttcacctagatccttttaaattaaaaatgaagttttaaatcaatctaaagtata
tatgagtaaacttggtctgacagttaccaatgcttaatcagtgaggcacctatctcagcgatctgtctatttcgttcatccata
gttgcctgactccccgtcgtgtagataactacgatacgggagggcttaccatctggccccagtgctgcaatgataccgcga
gacccacgctcaccggctccagatttatcagcaataaaccagccagccggaagggccgagcgcagaagtggtcctgcaa
ctttatccgcctccatccagtctattaattgttgccgggaagctagagtaagtagttcgccagttaatagtttgcgcaacgttg
ttgccattgctacaggcatcgtggtgtcacgctcgtcgtttggtatggcttcattcagctccggttcccaacgatcaaggcga
gttacatgatcccccatgttgtgcaaaaaagcggttagctccttcggtcctccgatcgttgtcagaagtaagttggccgcagt
gttatcactcatggttatggcagcactgcataattctcttactgtcatgccatccgtaagatgcttttctgtgactggtgagtac
tcaaccaagtcattctgagaatagtgtatgcggcgaccgagttgctcttgcccggcgtcaatacgggataataccgcgcca
catagcagaactttaaaagtgctcatcattggaaaacgttcttcggggcgaaaactctcaaggatcttaccgctgttgagat
ccagttcgatgtaacccactcgtgcacccaactgatcttcagcatcttttactttcaccagcgtttctgggtgagcaaaaaca
ggaaggcaaaatgccgcaaaaaagggaataagggcgacacggaaatgttgaatactcatactcttcctttttcaatattat
tgaagcatttatcagggttattgtctcatgagcggatacatatttgaatgtatttagaaaaataaacaaataggggttccgc
gcacatttccccgaaaagtgccacctgac
The vector LeGO-MCS-GSG-T2A-eGFP is set forth below as SEQ ID NO: 19:
(SEQ ID NO: 19)
gtcgacggatcgggagatctcccgatcccctatggtgcactctcagtacaatctgctctgatgccgcatagttaag
ccagtatctgctccctgcttgtgtgttggaggtcgctgagtagtgcgcgagcaaaatttaagctacaacaaggcaaggcttga
ccgacaattgcatgaagaatctgcttagggttaggcgttttgcgctgcttcgcgatgtacgggccagatatacgcgttgacatt
gattattgactagttattaatagtaatcaattacggggtcattagttcatagcccatatatggagttccgcgttacataacttacgg
taaatggcccgcctggctgaccgcccaacgacccccgcccattgacgtcaataatgacgtatgttcccatagtaacgccaat
agggactttccattgacgtcaatgggtggagtatttacggtaaactgcccacttggcagtacatcaagtgtatcatatgccaag
tacgccccctattgacgtcaatgacggtaaatggcccgcctggcattatgcccagtacatgaccttatgggactttcctacttg
gcagtacatctacgtattagtcatcgctattaccatggtgatgcggttttggcagtacatcaatgggcgtggatagcggtttgac
tcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagcgcgttttgcctgt
actgggtctctctggttagaccagatctgagcctgggagctctctggctaactagggaacccactgcttaagcctcaataaag
cttgccttgagtgcttcaagtagtgtgtgcccgtctgttgtgtgactctggtaactagagatccctcagacccttttagtcagtgt
ggaaaatctctagcagtggcgcccgaacagggacttgaaagcgaaagggaaaccagaggagctctctcgacgcaggact
cggcttgctgaagcgcgcacggcaagaggcgaggggcggcgactggtgagtacgccaaaaattttgactagcggaggct
agaaggagagagatgggtgcgagagcgtcagtattaagcgggggagaattagatcgcgatgggaaaaaattcggttaag
gccagggggaaagaaaaaatataaattaaaacatatagtatgggcaagcagggagctagaacgattcgcagttaatcctgg
cctgttagaaacatcagaaggctgtagacaaatactgggacagctacaaccatcccttcagacaggatcagaagaacttag
atcattatataatacagtagcaaccctctattgtgtgcatcaaaggatagagataaaagacaccaaggaagctttagacaagat
agaggaagagcaaaacaaaagtaagaccaccgcacagcaagcggccggccgcgctgatcttcagacctggaggagga
gatatgagggacaattggagaagtgaattatataaatataaagtagtaaaaattgaaccattaggagtagcacccaccaagg
caaagagaagagtggtgcagagagaaaaaagagcagtgggaataggagctttgttccttgggttcttgggagcagcagga
agcactatgggcgcagcgtcaatgacgctgacggtacaggccagacaattattgtctggtatagtgcagcagcagaacaat
ttgctgagggctattgaggcgcaacagcatctgttgcaactcacagtctggggcatcaagcagctccaggcaagaatcctg
gctgtggaaagatacctaaaggatcaacagctcctggggatttggggttgctctggaaaactcatttgcaccactgctgtgcc
ttggaatgctagttggagtaataaatctctggaacagatttggaatcacacgacctggatggagtgggacagagaaattaaca
attacacaagcttaatacactccttaattgaagaatcgcaaaaccagcaagaaaagaatgaacaagaattattggaattagata
aatgggcaagtttgtggaattggtttaacataacaaattggctgtggtatataaaattattcataatgatagtaggaggcttggta
ggtttaagaatagtttttgctgtactttctatagtgaatagagttaggcagggatattcaccattatcgtttcagacccacctccca
accccgaggggacccgacaggcccgaaggaatagaagaagaaggtggagagagagacagagacagatccattcgatt
agtgaacggatcggcactgcgtgcgccaattctgcagacaaatggcagtattcatccacaattttaaaagaaaaggggggat
tggggggtacagtgcaggggaaagaatagtagacataatagcaacagacatacaaactaaagaattacaaaaacaaattac
aaaaattcaaaattttcgggtttattacagggacagcagagatccagtttggttagtaccgggcccgctctagtcgaggtcga
cggtatcgataagctcgcttcacgagattccagcaggtcgagggacctaataacttcgtatagcatacattatacgaagttata
ttaagggttccaagcttaagcggccgctgaaagaccccacctgtaggtttggcaagctagctgcagtaacgccattttgcaa
ggcatggaaaaataccaaaccaagaatagagaagttcagatcaagggcgggtacatgaaaatagctaacgttgggccaaa
caggatatctgcggtgagcagtttcggccccggcccggggccaagaacagatggtcaccgcagttteggccccggcccg
aggccaagaacagatggtccccagatatggcccaaccctcagcagtttcttaagacccatcagatgtttccaggctccccca
aggacctgaaatgaccctgcgccttatttgaattaaccaatcagcctgcttctcgcttctgttegcgcgcttctgcttcccgagct
ctataaaagagctcacaacccctcactcggcgcgccagtcctccgattgactgagtcgcccGGATCCGCCACC
ATGGCTAAGCTAGGAAGCGGAGAGGGCAGAGGCAGTCTGCTGACATGCGG
TGACGTGGAAGAGAATCCCGGCCCTgtgagcaagggcgaggagctgttcaccggggggtgccc
atcctggtcgagctggacggcgacgtaaacggccacaagttcagcgtgtccggcgagggcgagggcgatgccacctac
ggcaagctgaccctgaagttcatctgcaccaccggcaagctgcccgtgccctggcccaccctcgtgaccaccctgacctac
ggcgtgcagtgcttcagccgctaccccgaccacatgaagcagcacgacttcttcaagtccgccatgcccgaaggctacgtc
caggagcgcaccatcttcttcaaggacgacggcaactacaagacccgcgccgaggtgaagttcgagggcgacaccctgg
tgaaccgcatcgagctgaagggcatcgacttcaaggaggacggcaacatcctggggcacaagctggagtacaactacaa
cagccacaacgtctatatcatggccgacaagcagaagaacggcatcaaggtgaacttcaagatccgccacaacatcgagg
acggcagcgtgcagctcgccgaccactaccagcagaacacccccatcggcgacggccccgtgctgctgcccgacaacc
actacctgagcacccagtccgccctgagcaaagaccccaacgagaagcgcgatcacatggtcctgctggagttcgtgacc
gccgccgggatcactctcggcatggacgagctgtacaagTAAGAATTCgtcgagggacctaataacttcgtatagc
atacattatacgaagttatacatgtttaagggttccggttccactaggtacaattcgatatcaagcttatcgataatcaacctctgg
attacaaaatttgtgaaagattgactggtattcttaactatgttgctccttttacgctatgtggatacgctgctttaatgcctttgtatc
atgctattgcttcccgtatggctttcattttctcctccttgtataaatcctggttgctgtctctttatgaggagttgtggcccgttgtca
ggcaacgtggcgtggtgtgcactgtgtttgctgacgcaacccccactggttggggcattgccaccacctgtcagctcctttcc
gggactttcgctttccccctccctattgccacggcggaactcatcgccgcctgccttgcccgctgctggacaggggctcggc
tgttgggcactgacaattccgtggtgttgtcggggaaatcatcgtcctttccttggctgctcgcctgtgttgccacctggattctg
cgcgggacgtccttctgctacgtcccttcggccctcaatccagcggaccttccttcccgcggcctgctgccggctctgcggc
ctcttccgcgtcttcgccttcgccctcagacgagtcggatctccctttgggccgcctccccgcatcgataccgtcgacctcgat
cgagacctagaaaaacatggagcaatcacaagtagcaatacagcagctaccaatgctgattgtgcctggctagaagcacaa
gaggaggaggaggtgggttttccagtcacacctcaggtacctttaagaccaatgacttacaaggcagctgtagatcttagcc
actttttaaaagaaaaggggggactggaagggctaattcactcccaacgaagacaagatatccttgatctgtggatctaccac
acacaaggctacttccctgattggcagaactacacaccagggccagggatcagatatccactgacctttggatggtgctaca
agctagtaccagttgagcaagagaaggtagaagaagccaatgaaggagagaacacccgcttgttacaccctgtgagcctg
catgggatggatgacccggagagagaagtattagagtggaggtttgacagccgcctagcatttcatcacatggcccgagag
ctgcatccggactgtactgggtctctctggttagaccagatctgagcctgggagctctctggctaactagggaacccactgct
taagcctcaataaagcttgccttgagtgcttcaagtagtgtgtgcccgtctgttgtgtgactctggtaactagagatccctcaga
cccttttagtcagtgtggaaaatctctagcagcatgtgagcaaaaggccagcaaaaggccaggaaccgtaaaaaggccgc
gttgctggcgtttttccataggctccgcccccctgacgagcatcacaaaaatcgacgctcaagtcagaggtggcgaaaccc
gacaggactataaagataccaggcgtttccccctggaagctccctcgtgcgctctcctgttccgaccctgccgcttaccggat
acctgtccgcctttctcccttcgggaagcgtggcgctttctcatagctcacgctgtaggtatctcagttcggtgtaggtcgttcg
ctccaagctgggctgtgtgcacgaaccccccgttcagcccgaccgctgcgccttatccggtaactatcgtcttgagtccaac
ccggtaagacacgacttatcgccactggcagcagccactggtaacaggattagcagagcgaggtatgtaggcggtgctac
agagttcttgaagtggtggcctaactacggctacactagaagaacagtatttggtatctgcgctctgctgaagccagttacctt
cggaaaaagagttggtagctcttgatccggcaaacaaaccaccgctggtagcggtggtttttttgtttgcaagcagcagatta
cgcgcagaaaaaaaggatctcaagaagatcctttgatcttttctacggggtctgacgctcagtggaacgaaaactcacgttaa
gggattttggtcatgagattatcaaaaaggatcttcacctagatccttttaaattaaaaatgaagttttaaatcaatctaaagtatat
atgagtaaacttggtctgacagttaccaatgcttaatcagtgaggcacctatctcagcgatctgtctatttcgttcatccatagttg
cctgactccccgtcgtgtagataactacgatacgggagggcttaccatctggccccagtgctgcaatgataccgcgagacc
cacgctcaccggctccagatttatcagcaataaaccagccagccggaagggccgagcgcagaagtggtcctgcaactttat
ccgcctccatccagtctattaattgttgccgggaagctagagtaagtagttcgccagttaatagtttgcgcaacgttgttgccatt
gctacaggcatcgtggtgtcacgctcgtcgtttggtatggcttcattcagctccggttcccaacgatcaaggcgagttacatga
tcccccatgttgtgcaaaaaagcggttagctccttcggtcctccgatcgttgtcagaagtaagttggccgcagtgttatcactc
atggttatggcagcactgcataattctcttactgtcatgccatccgtaagatgcttttctgtgactggtgagtactcaaccaagtc
attctgagaatagtgtatgcggcgaccgagttgctcttgcccggcgtcaatacgggataataccgcgccacatagcagaact
ttaaaagtgctcatcattggaaaacgttcttcggggcgaaaactctcaaggatcttaccgctgttgagatccagttcgatgtaac
ccactcgtgcacccaactgatcttcagcatcttttactttcaccagcgtttctgggtgagcaaaaacaggaaggcaaaatgcc
gcaaaaaagggaataagggcgacacggaaatgttgaatactcatactcttcctttttcaatattattgaagcatttatcagggtta
ttgtctcatgagcggatacatatttgaatgtatttagaaaaataaacaaataggggttccgcgcacatttccccgaaaagtgcc
acctgac

The above vector LeGO-MCS-GSG-T2A-eGFP has been generated by replacing the sequence of the LeGO-G2 vector (Addgene 25917) with the following sequence between the BamH1 and EcoR1 restriction sites:

(SEQ ID NO: 22)
GGATCCGCCACCATGGCTAAGCTAGGAAGCGGAGAGGGCAGAGGCAGTCT
GCTGACATGCGGTGACGTGGAAGAGAATCCCGGCCCTgtgagcaagggcg
aggagctgttcaccggggtggtgcccatcctggtcgagctggacggcgac
gtaaacggccacaagttcagcgtgtccggcgagggcgagggcgatgccac
ctacggcaagctgaccctgaagttcatctgcaccaccggcaagctgcccg
tgccctggcccaccctcgtgaccaccctgacctacggcgtgcagtgcttc
agccgctaccccgaccacatgaagcagcacgacttcttcaagtccgccat
gcccgaaggctacgtccaggagcgcaccatcttcttcaaggacgacggca
actacaagacccgcgccgaggtgaagttcgagggcgacaccctggtgaac
cgcatcgagctgaagggcatcgacttcaaggaggacggcaacatcctggg
gcacaagctggagtacaactacaacagccacaacgtctatatcatggccg
acaagcagaagaacggcatcaaggtgaacttcaagatccgccacaacatc
gaggacggcagcgtgcagctcgccgaccactaccagcagaacacccccat
cggcgacggccccgtgctgctgcccgacaaccactacctgagcacccagt
ccgccctgagcaaagaccccaacgagaagcgcgatcacatggtcctgctg
gagttcgtgaccgccgccgggatcactctcggcatggacgagctgtacaa
gTAAGAATTC

Generation of PD-L1⁺ and PD-L1⁻ Target Cell Lines

Raji cells were transfected with PD-L1 plasmid (GenScript, OHu22144), using the Amaxa Cell Line Nucleofector kit V (Lonza, VCA-1003) according to the manufacturer's recommendations. PD-L1 protein was knocked out in 786-0 cells with CRISPR-Cas9 technology according to previously published protocols [21]. Two different guide RNAs were used to generate KO1 (guide RNA sequence: TACCGCTGCATGATCAGCTATGG) (SEQ ID NO: 20) and KO2 (guide RNA sequence: TACCATACTCTACCACATATAGG) (SEQ ID NO: 21) to ensure obtained results were due to PD-L1 KO and not unwanted off-target alterations associated with CRISPR technology.

### Production of Lentiviral Vectors

Lentiviruses were generated by calcium-phosphate based transfection (Sigma, CAPHOS-1KT) of either 1×10⁶ HEK293FT cells (CRISPR plasmids) or 14×10⁶ HEK293FT cells (PD1-CSR plasmids) according to the manufacturer's recommendations. Briefly, the plasmids of interest were co-transfected with the envelope plasmid pCMV-VSV-G and two packaging plasmids, pDMLg/pPRE and pRSV-Rev to produce VSV-G-pseudotyped lentiviruses. For the transduction of NK cells, lentiviruses were concentrated prior to freezing with the Lenti-X™ concentrator according to the manufacturer's recommendations (Takara Bio, 631232). All lentiviruses were titrated on HEK293FT cells. Briefly, 5×10⁴ cells per well of a 24-well plate were seeded in medium, containing different amounts of concentrated virus, in the presence of 8 μg/μl protamine sulfate (Sigma-Aldrich P3369-10G). Cells were spinoculated for one hour at 1000×g and 32° C. after which incubation for 6 hours at 37° C. and 5% CO₂ followed. Protamine-sulfate containing medium was then replaced with fresh medium. Green fluorescent protein (GFP) percentage was analysed by flow cytometry three days post-transduction. Lentiviral titer was calculated with the formula

$$\frac{\% of GFP positive cells \times 50.}{viral supernatant in ml}$$

### Lentiviral Transduction of NK-92, pNK Cells and 786-0) Cells

NK cells were transduced as described previously [24]. pNK cells were isolated from healthy donor PBMCs. Briefly, cells were seeded at 5×10⁵ cells/ml in viral supernatant at an MOI of 4 (NK-92) or 15 (pNK) in the presence of (5Z)-7-Oxozeanol (Biotechne) and 8 μg/μl protamine sulfate (Sigma-Aldrich P3369-10G). Cells were spinoculated for one hour at 1,000×g and 32° C. after which incubation for five hours at 37° C. and 5% CO₂ followed. Protamine-sulfate containing medium was then replaced with fresh medium, containing IL-2 at a final concentration of 500 U/ml (NK-92) or 1000 U/ml (pNK) (R&D 202-IL-500). 786-0 cells were plated at a density of 3,300 cells/cm² in the presence of viral supernatant and 8 μg/μl protamine sulfate (Sigma-Aldrich P3369-10G). Cells were spinoculated for one hour at 800×g and 32° C. after which incubation for five hours at 37° C. and 5% CO₂ followed. Medium was then replaced with fresh medium. GFP, PD1 and PD-L1 expression were analysed three days after transduction. Cells were sorted using BD FACS AriaFusion.

### Flow Cytometry

The following mAb were used for flow cytometry analysis: CD56 (clone NCAM1), CD16 (clone 3G8) CD3 (clone SK3), CD11b (clone ICRF44), CD14 (clone MOP9) PD1 (clone EH12.1), PD-L1 (clone MIH1), PD-L2 (clone MIH18), CD138 (clone MI15) MICA/B (clone 6D4), CD155 (clone TX24), NKG2A (clone 131411), NKp44 (clone p44-8), TIM3 (clone 7D3), TIGIT (clone 741182), DNAM1 (clone DX11) from BD Biosciences; HLA-ABC (clone W6/32), CD38 (clone HIT2), NKp30 (clone p30-15), NKp46 (9E2/NKp46), LAG3 (clone 11C3C65), NKG2D (clone 1D11), CD112 (clone TX31) from BioLegend; CD158a/h/g (clone HP-MA4), CD158e (clone DX9) from Thermofisher, CD158B (clone GL183) from Invitrogen, ULBP256 (clone 165903) from R&D. All antibodies were titrated prior to usage. Briefly, cells were collected and washed once in PBS. Cells were stained with Aqua live dead cell staining (ThermoFisher) for 20 minutes at 4° C., in the dark. Cells were washed once with PBS, containing 2% FBS. Surface staining was performed for 25 minutes at 4° C., in the dark. Cells were washed with PBS, containing 2% FBS, centrifuged and fixed with 1% paraformaldehyde for 10 minutes. Acquisition was performed the following day with Beckman Coulter Cytoflex flow cytometers. Analysis was performed with FlowJo analysis software version 10. Gates were—unless otherwise specified—placed based on the unstained control or FMO (fluorescence minus one).

### NK Cell Degranulation Assay and Evaluation of IFNγ and TNF Intracellular Staining

0.03×10⁶ 786-O cells were seeded in a flat 96-well plate 24 hours before the assay to allow cells to attach. 0.15×10⁶ NK-92 or pNK cells were co-incubated with either 0.15×10⁶ Raji cells or 786-O cells in a final volume of 200 μl at 37° C. and 5% CO₂ for four hours in the presence of CD107a antibody (BioLegend, clone H4A3). Where indicated, Rituximab was added to the co-culture at a final concentration of 2.5 μg/ml. As controls, 0.15×10⁶ NK-92 or pNK cells were incubated alone or with phorbol 12-myristate 13-acetate (PMA) and ionomycin (0.5 μg/mL, Sigma-Aldrich), together with CD107a antibody for 4 hours. After one hour of incubation, monensin (GolgiStop, BD Biosciences) was added to cultures to inhibit protein transportation. Subsequently, surface staining with CD56 (clone NCAM16.2), CD16 (clone 3G8), CD3 (clone UCHT1) and PD1 (clone EH12.1) was performed for 25 min at 4° C., in the dark. For intracellular staining of IFNγ (clone B27) and TNF (clone MAb11) (all from BD Bioscience) cells were washed with PBS followed by fixation and permeabilization with cytofix/cytoperm (BD Biosciences). Cell were incubated for 30 min at RT with intracellular antibodies. Cells were washed and resuspended in PBS. Acquisition was performed with Beckman Coulter Cytoflex or BD Symphony flow cytometers. Analysis was performed with FlowJo analysis software version 10. Gates were placed on the unstimulated samples for the readout of CD107a, IFNγ and TNF.

### Chromium Release Assay

NK cell cytotoxicity was measured in a standard ⁵¹Cr-release assay against tumor target cells. Briefly, target cells were labeled with 100 μL sodium chromate (PerkinElmer) for one hour at 37° C., after which they were washed three times with PBS. NK cells were mixed with the labeled target cells at different effector to target ratios and incubated for four hours. 20 μL of the supernatant was transferred to LumaPlate-96 and subsequently analyzed with a MicroBeta2 counter (PerkinElmer).

### Live Cell Imaging Assays

Live cell imaging was performed as recently described [25]. Briefly, 3×10⁴ PD-L1⁺ 786-O WT or PD-L1⁻ 786-O KO cells that were previously transduced to express the fluorescent protein tdTomato were seeded per well in a low-attachment 96-well plate and incubated at 37° C., 5% CO₂ for 72 hours to allow spheroids to form spontaneously. Prior to analysis, 3×10³ NK-92 or pNK cells were added to the culture. The number of killed target cells was monitored by imaging every four hours over 48 hours (NK-92) to seven days (pNK) using an IncuCyte S3 Live Cell Analysis System (Sartorius). Percent of killing was quantified as decrease of red intensity and normalized to the red fluorescence intensity at the beginning of the assay with the formula

$$\frac{red cell count at timepoint x}{red cell count at timepoint i} .$$

### Proliferation Assays

NK-92 and pNK cells were labelled with Cell Trace Violet (ThermoFisher) according to the manufacturer's recommendations. Cells were cultured alone or co-cultured in the presence of PD-L1⁺ 786-O WT or PD-L1⁻ 786-O KO cells at an effector to target ratio of 1:1. Acquisition was performed with Beckman coulter Cytoflex flow cytometers. Analysis was performed with FlowJo analysis software version 10.

### Statistical Analysis

The Student's t test was used to compare the means of two groups. Two-way ANOVA test was used to compare the means between several groups. p<0.05 was determined as statistically significant (*), p<0.01 (**), p<0.001 (***), p<0.0001 (****) as statistically highly significant. Statistical analysis was performed with GraphPad Prism software version 9 (GraphPad, La Jolla, USA).

### Results

### Expression of PD1-Based Chimeric Switch Receptors in NK-92 Cells

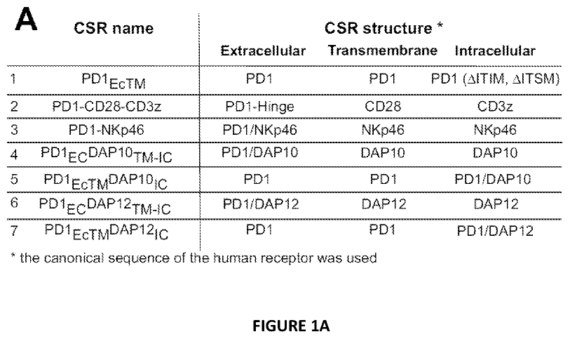

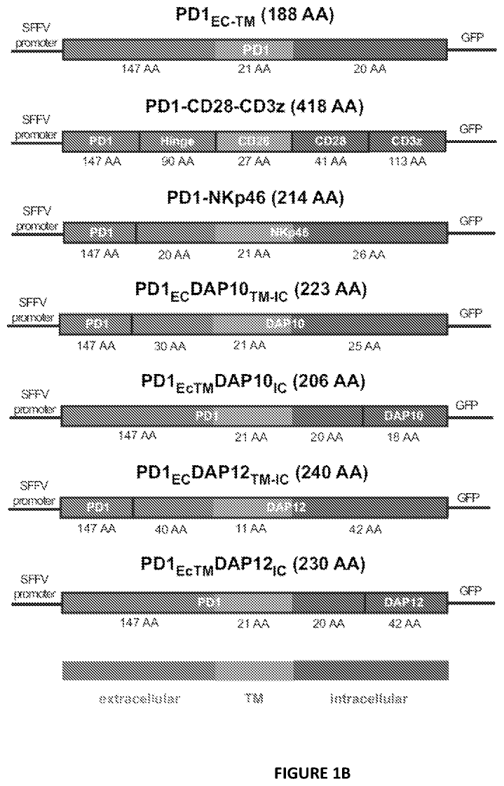

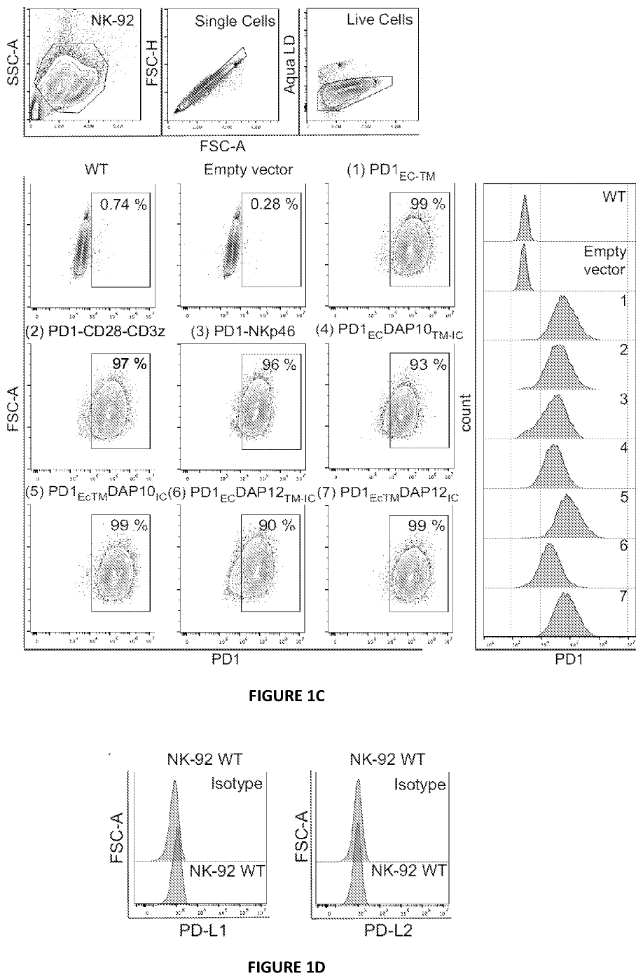

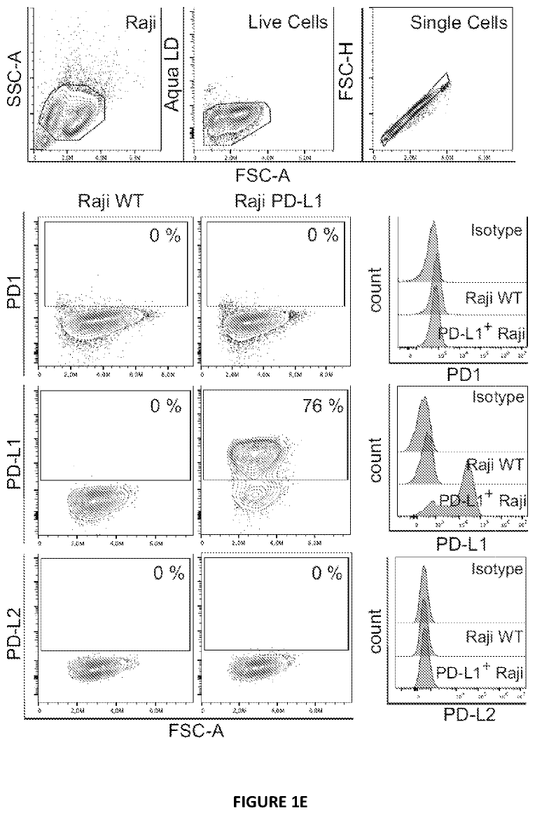

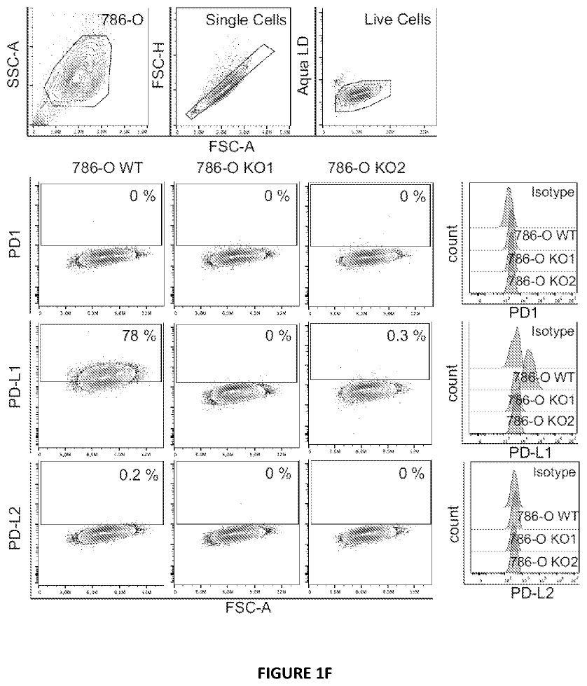

Initially, six different CSR constructs were generated with the purpose of determining optimal signaling in NK cells. All CSR expressed the unmodified human PD1 extracellular domain fused with various activating intracellular domains. Specifically, DAP10, DAP12, CD3ζ and NKp46 were utilized. Furthermore, a control coding for a truncated, signaling-deficient PD1 construct was generated (FIG. 1A, B). PD1 surface expression among the untransduced or empty vector transduced NK-92 cell lines remained below 2% of the total population. After sorting, all other transduced cell lines stably expressed the transgenes as confirmed by positive PD1 staining. As expected, expression levels differed between the constructs (FIG. 1 C). Importantly, neither PD-L1 nor PD-L2 expression was detected in NK-92 wildtype (WT) cells (FIG. 1 D).

FIG. 1A-D are Table and Vector Maps showing that PD1-based chimeric switch receptors are stably expressed in NK-92 cells. FIGS. 1A-B show Table and vector maps depicting the design of the truncated PD1 receptor (PD1_{EcTM}) and six chimeric switch receptors (CSR) with different signaling domains. FIG. 1C shows NK-92 cells containing different PD1-CSR and sorted for positive PD1 surface staining. FIG. 1 D shows PD-L1 and PD-L2 expression on NK-92 cells. FIG. 1E shows PD1, PD-L1 and PD-L2 expression on PD-L1⁺ Raji and PD-L1⁻ Raji WT cells. FIG. 1F shows PD1, PD-L1 and PD-L2 expression on PD-L1⁺ 786-O WT and PD-L1⁻ 786-O KO cells.

### Generation of Target Cell Lines

To study the function of the PD1-CSR⁺ NK-92 cell lines, PD-L1⁺ and PD-L1⁻ target cell lines were generated. As target cell lines 786-O and Raji cells were chosen, with the first expressing PD-L1 and the latter being devoid of PD-L1. The target cell lines were chosen due to their different potential to activate NK cells. Based on these cell lines, PD-L1 knock-out 786-O (786-O KO) cell lines and a PD-L1⁺ Raji cell line were generated. Hereafter, the target cell lines are referred to as PD-L1⁺ 786-O WT, PD-L1⁻ 786-O KO1, PD-L⁻ 786-O KO2, PD-L1⁺ Raji and PD-L1⁻ Raji WT. Neither of the target cell lines expressed PD1 or PD-L2 (FIG. 1 E, F). FIG. 1E shows that PD1, PD-L1 and PD-L2 expression on PD-L1⁺ Raji and PD-L1⁻ Raji WT cells. FIG. 1 F shows that PD1, PD-L1 and PD-L2 expression on PD-L1⁺ 786-O WT and PD-L1⁻ 786-O KO cells.

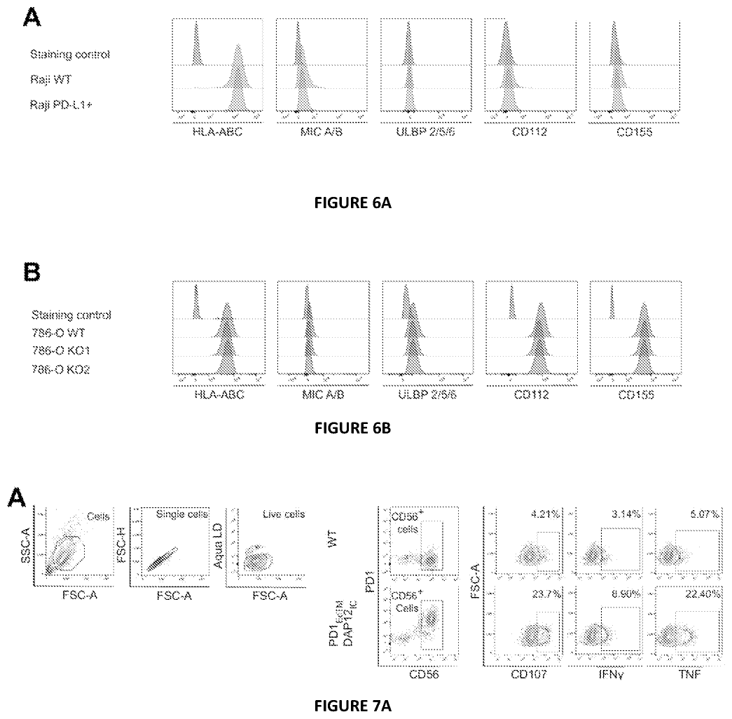

The genetic modification of the target cell lines did not alter the expression of other ligands for activating NK cell receptors (FIG. 6A, B). FIGS. 6A-B show flow cytometry plots depict the expression of ligands for activating NK cell receptors on Raji WT and PD-L1⁺ Raji cells as well as 786-O WT, 786-O KO1 and 786-O KO2 cell lines compared to a staining control.

### PD1-CSR Transduced NK-92 Cell Lines Show Superior Degranulation and Cytokine Secretion

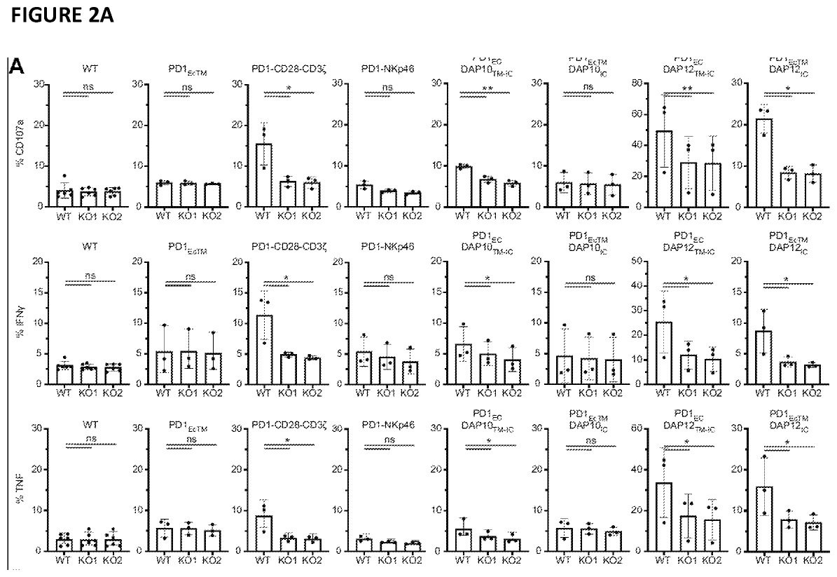

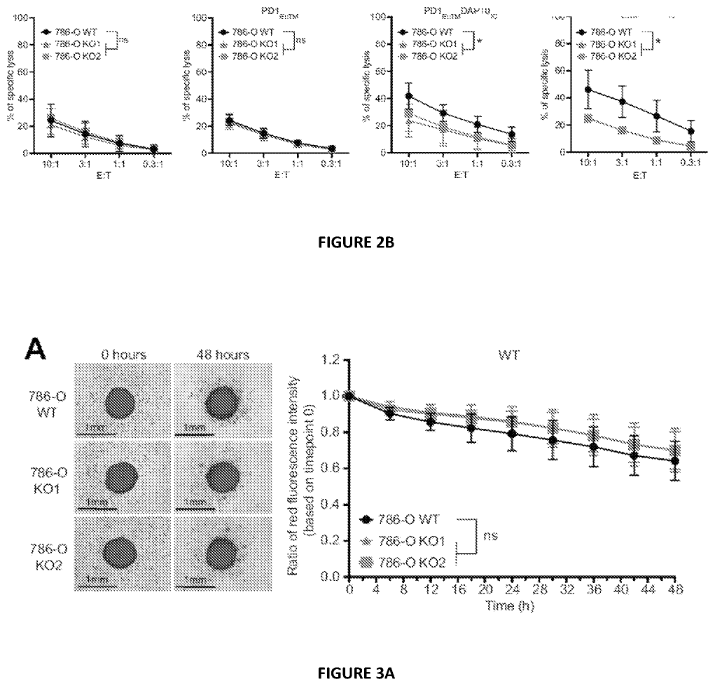

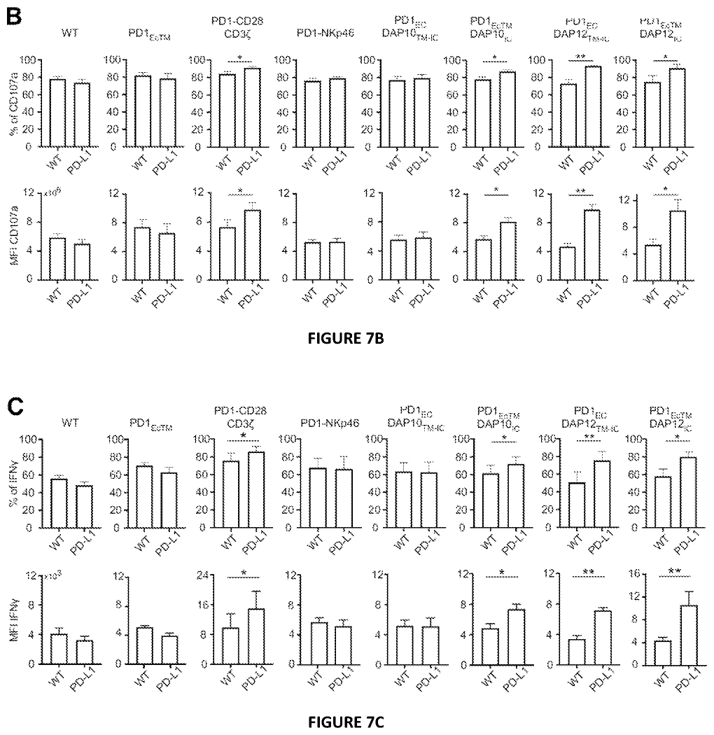

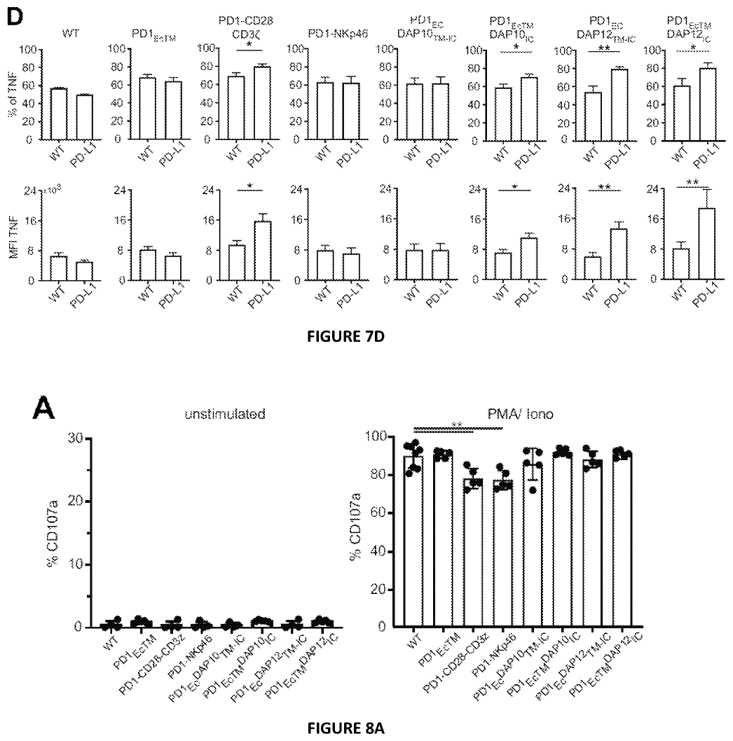

To evaluate the induction of degranulation and cytokine expression, CD107a, IFNγ and TNF expression by all generated PD1-CSR⁺ NK-92 cell lines and controls was assessed against PD-L1⁺ 786-O WT and PD-L1⁻ 786-O KO cell lines (FIG. 2A, FIG. 7A). Generally, 786-O cells are resistant to NK cell mediated cytotoxicity and the PD1-CSR⁺ NK-92 cells were tested for their ability to circumvent the resistance. In line with this, CD107a, IFNγ and TNF expression by NK-92 WT and PD1_{EcTM}⁺ NK-92 remained below 10% against both PD-L1⁺ 786-O WT and PD-L1⁻ 786-O KO cell lines, with no significant differences between the three target cell lines. FIG. 2A shows the percentage of CD107a, IFNγ and TNF by different PD1-CSR⁺ NK-92 cells against PD-L1⁺ 786-O WT and two PD-L1⁻ 786-O KO cell lines. Each data point represents the mean (+/−SD) of three independent experiments performed in triplicates. FIG. 7A shows flow cytometry plots depicting the gating strategy for degranulation assays.

The PD1-CD28-CD3ζ and PD1_{EcTM}DAP12_{IC} transduced NK-92 cell lines showed a three- to four-fold increase in CD107a expression and corresponding increase in IFNγ and TNF expression against PD-L1⁺ 786-O WT compared to both PD-L1⁻ 786-O KO cell lines. CD107a, IFNγ and TNF production was 1.5-fold higher by PD1_{EC}DAP10_{TM-IC}⁺ NK-92 against PD-L1⁺ 786-O WT cells. CD107a, IFNγ and TNF expression by PD1_{EC}DAP12_{TM-IC}⁺ NK-92 cells showed the highest degranulation against PD-L1⁺ 786-O WT cells among the PD1-CSR⁺ NK-92 cell lines, approaching 50% (+/−12%), 30% (+/−8.5%) and 30% (+/−8.5%), respectively. Notably, PD1-NKp46 and PD1_{EcTM}DAP10_{IC} expressing NK-92 cell lines did not show increased CD107a nor IFNγ or TNF expression against PD-L1⁺ 786-O WT compared to PD-L1⁻ 786-O KO cell lines. To conclude, PD1-CD28-CD3ζ, PD1_{Ec}DAP10_{TM-IC}⁺, PD1_{Ec}DAP12_{TM-IC}⁺ and PD1_{EcTM}DAP12_{IC}⁺ NK-92 cells increased degranulation and cytokine expression against PD-L1⁺ 786-O WT cells which are inherently resistant to NK cell mediated cytotoxicity.

### PD1-CSR Transduced NK-92 Cell Lines Show Superior Degranulation and Cytokine Secretion Against PD-L1⁺ Raji Cells

To extend these results to a cell line that is already highly susceptible to NK cell killing, all generated PD1-CSR⁺ NK-92 cell lines were assessed against PD-L1⁺ Raji cells and PD-L1⁻ Raji WT cells (FIG. 7 B-D). FIGS. 7B-D show the percentage and MFI of CD107a (B), IFNγ (C) and TNF (D) by different PD1-CSR⁺ NK-92 cells against PD-L1⁺ Raji cells and PD-L1⁻ Raji WT cells. Each data point represents the mean (+/−SD) of three independent experiments performed in triplicates. Statistical significance (* p<0.05; ** p<0.01) was determined with a two-way ANOVA test. NK92 WT and PD1_{EcTM}⁺ NK92 cells showed a high CD107a, IFNγ and TNF expression against both PD-L1⁻ Raji WT cells and PD-L1⁺ Raji cells with values reaching up to 80% (+/−7%), 70% (+/−6%) and 70% (+/−1.2%), respectively. Despite the high baseline values, the expression of CD107a, IFNγ and TNF by PD1-CD28-CD3ζ, PD1_{EcTM}DAP10_{IC}⁺, PD1_{Ec}DAP12_{TM-IC}⁺ and PD1_{EcTM}DAP12_{IC}⁺ NK-92 cells was significantly higher against PD-L1⁺ Raji cells compared to PD-L1⁻ Raji WT cells. PD1-NKp46 and PD1_{Ec}DAP10_{TM-IC} expressing NK-92 cell lines did not increase CD107a, IFNγ or TNF expression against PD-L1⁺ Raji cells compared to PD-L1⁻ Raji WT cells. Taken together, PD1-CD28-CD3ζ, PD1_{EcTM}DAP10_{IC}⁺, PD1_{Ec}DAP12_{TM-IC}⁺ and PD1_{EcTM}DAP12_{IC}⁺ NK-92 cells increased degranulation and cytokine expression against PD-L1⁺ Raji cells.

### Receptor-Independent Degranulation and Cytokine Production by PD1-CSR⁺ NK-92 Cells

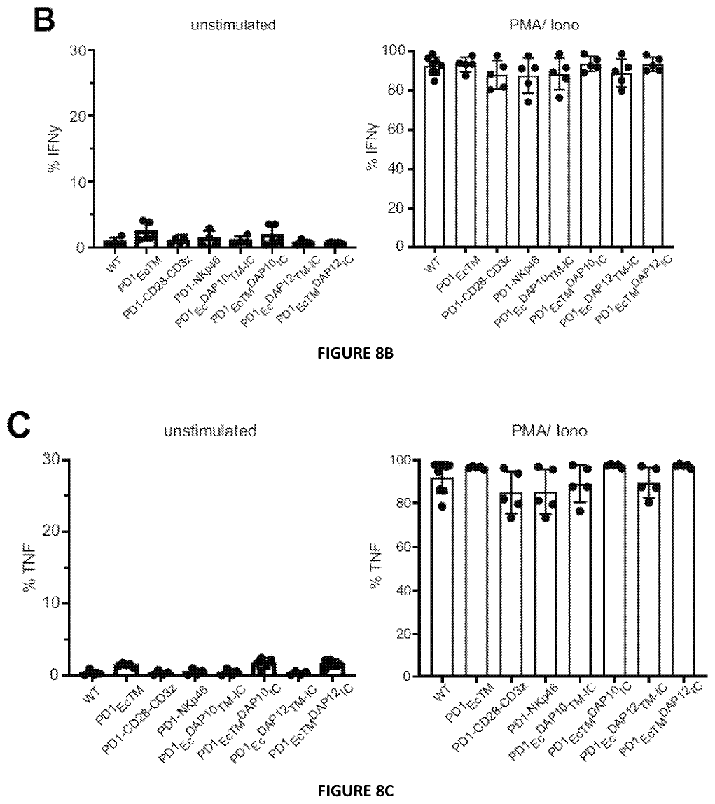

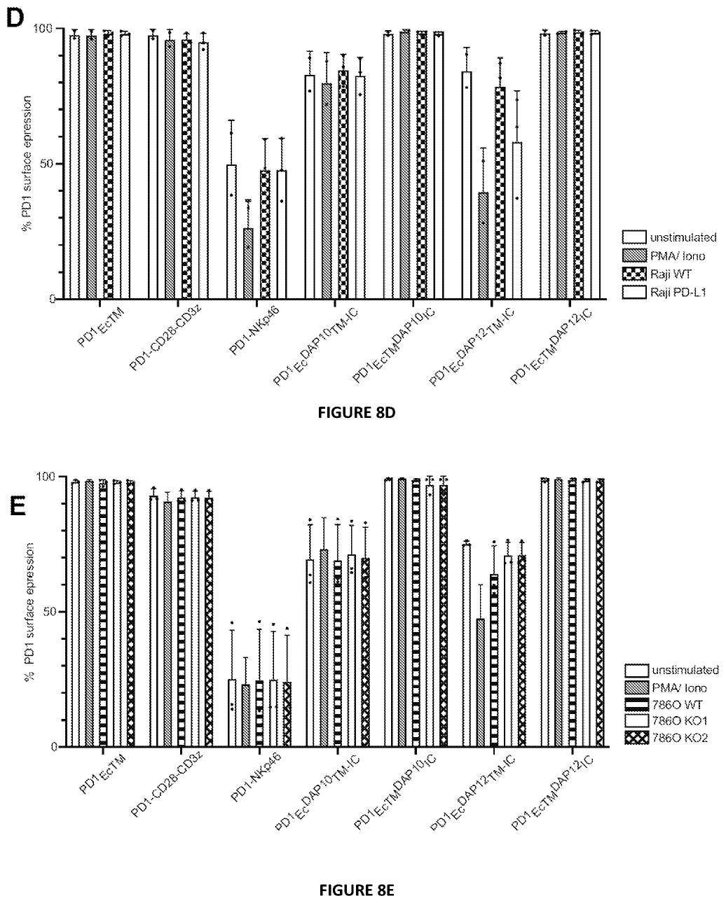

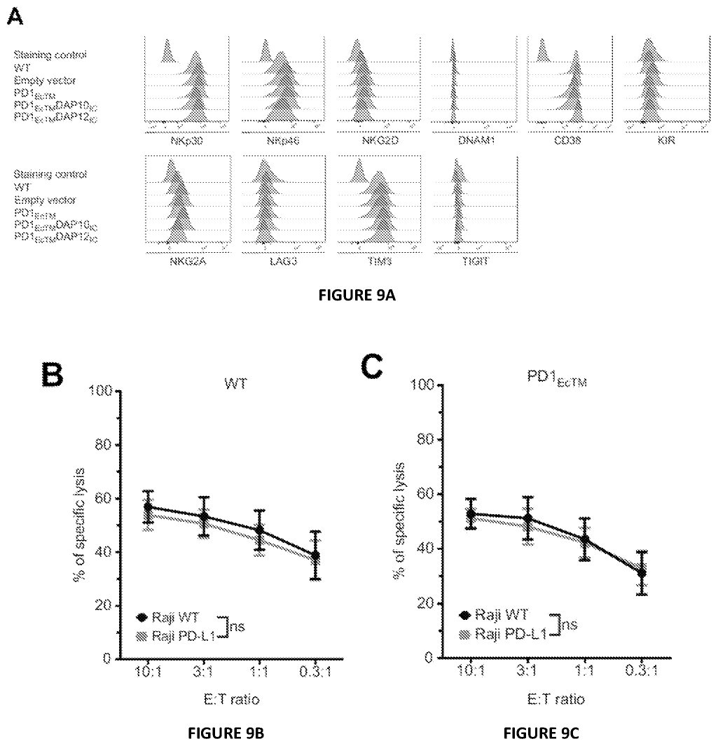

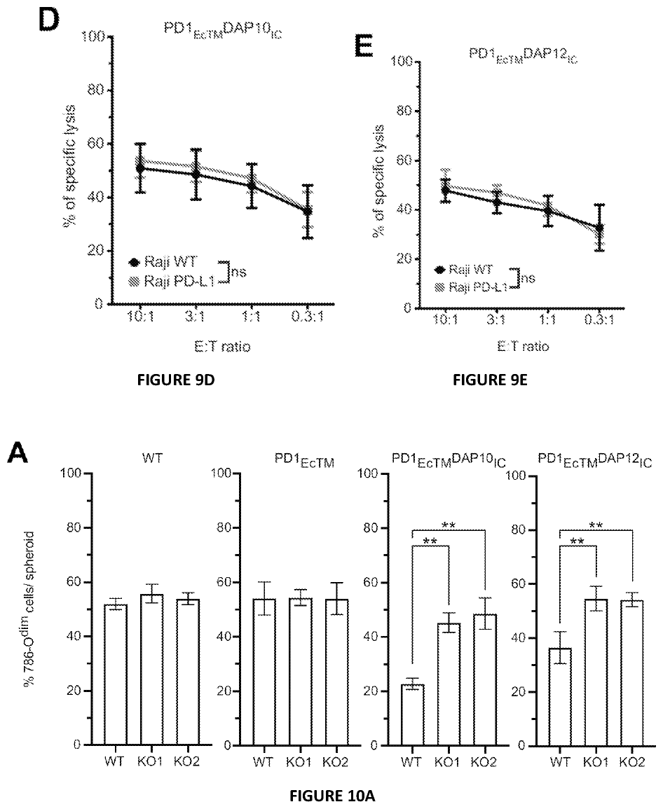

To confirm that the induction of CD107a, IFNγ and TNF expression is based on PD1-PD-L1 interaction, degranulation and cytokine expression by unstimulated or maximal chemically (PMA/Iono) stimulated PD1-CSR⁺ NK-92 cell lines was measured (FIG. 8A-C). FIGS. 8A-C show the percentage of CD107a (A), IFNγ (B) and TNF (C) expression of unstimulated or maximal chemically stimulated with phorbol-myristate-acetate (PMA) PD1-CSR⁺ NK-92 cells. No significant differences in CD107a, IFNγ or TNF expression by unstimulated PD1-CSR⁺ NK-92 cell lines was observed. However, chemical stimulation of PD1-CD28-CD3ζ⁺ and PD1-NKp46⁺ NK-92 cells resulted in reduced CD107a, but not IFNγ or TNF expression, compared to NK-92 WT cells. The other PD1-CSR⁺ NK-92 cells did not show differences in CD107a, IFNγ or TNF expression after chemical stimulation compared to NK-92 WT cells. PD1 positivity decreased in the PD1-NKp46⁺ NK-92 cell line in the absence of stimulation as well as after chemical stimulation or target cell recognition. After chemical stimulation or recognition of PD-L1⁺ target cells, PD1 surface expression also decreased in the PD1_{Ec}DAP 12_{TM-IC} expressing cell line and remained stable for the other PD1-CSR⁺ NK-92 cell lines (FIG. 8D-E). FIGS. 8D-E show PD1 surface expression in PD1-CSR⁺ NK92 cells in unstimulated cells, maximal chemically stimulated (PMA) cells or during co-culture with target cells. Each data point represents the mean (+/−SD) of three independent experiments performed in duplicates. Statistical significance (* p<0.05; ** p<0.01) was determined with a two-way ANOVA test. Taken together, PD1_{EcTM}DAP10_{IC}⁺ and PD1_{EcTM}DAP12_{IC}⁺ NK-92 cells showed a stable PD1 surface staining, a higher CD107a, IFNγ and TNF expression upon PD-L1⁺ target cell recognition and no alteration in degranulation or cytokine secretion in the unstimulated or maximal chemically stimulated controls compared to NK-92 WT cells. Genetic modification did not alter the expression of other activating and inhibitory NK cell receptors on mock-transduced, PD1_{EcTM}, PD1_{EcTM}DAP10_{IC} and PD1_{EcTM}DAP 12_{IC} transduced NK92 cells (FIG. 9A) FIG. 9A shows flow ctometry plots depict the expression of activating and inhibitory NK cell receptors on NK92 WT and PD1-CSR⁺ NK92 cell lines compared to a staining control. Therefore, PD1_{EcTM}DAP10_{IC} and PD1_{EcTM}DAP 12_{IC} CSR were chosen for the following assays.

PD1_{EcTM}DAP10_{IC}⁺ and PD1_{EcTM}DAP12_{IC}⁺ NK-92 Cells Show Higher Killing of PD-L1⁺ 786-O WT cells

Since PD1_{EcTM}DAP10_{IC}⁺ and PD1_{EcTM}DAP12_{IC}⁺ NK-92 cells showed higher efficacy, we explored whether these cells would also directly kill tumor target cells. Killing of ⁵¹Cr labelled PD-L1⁺ 786-O WT or PD-L1⁻ 786-O KO cells by either NK-92 WT, PD1_{EcTM}⁺, PD1_{EcTM}DAP10_{IC}⁺ or PD1_{EcTM}DAP12_{IC}⁺ NK-92 cell lines was assessed at different effector to target (E:T) ratios (FIG. 2B). There was no significant difference in killing of PD-L1⁺ 786-O WT cells compared to both PD-L1⁻ 786-O KO cell lines by either NK-92 WT or PD1_{EcTM}⁺ NK92 cells. PD1_{EcTM}DAP10_{IC}⁺ NK-92 cells increased killing of PD-L1⁺ 786-O WT cells two-fold compared to both PD-L1⁻ 786-O KO cell lines. Similarly, PD1_{EcTM}DAP12_{IC}⁺ NK-92 cells increased killing of PD-L1⁺ 786-O WT cells by two-fold at higher E:T ratios and 3.5 fold at lower E:T ratios compared to PD-L1⁻ 786-O KO cells. FIG. 2B shows the killing of PD-L1⁺ 786-O WT versus PD-L1⁻ 786-O KO1 and PD-L1⁻ 786-O KO2 cells by NK-92 WT, PD1_{EcTM}³⁰, PD1_{EcTM}DAP10_{IC}⁺ or PD1_{EcTM}DAP12_{IC}⁺ NK-92 cells. Each data point represents the mean (+/−SD) of three independent experiments performed in quadruplets. Statistical significance (* p<0.05; ** p<0.01) was determined with a two-way ANOVA test. No significant difference in killing of PD-L1⁺ Raji compared to PD-L1⁻ Raji WT cells was observed by either of the PD1-CSR⁺ NK-92 cells (FIG. 9B-E). FIG. 9B shows flow ctometry plots depict the expression of activating and inhibitory NK cell receptors on NK92 WT and PD1-CSR⁺ NK92 cell lines compared to a staining control. FIGS. 9C-E show the killing of PD-L1⁺ Raji versus PD-L1⁻ Raji WT by NK-92 WT, PD1_{EcTM}⁺, PD1_{EcTM}DAP10_{IC}⁺ or PD1_{EcTM}DAP12_{IC}⁺ NK-92 cells. Each data point represents the mean (+/−SD) of three independent experiments performed in triplicates.

PD1_{EcTM}DAP10_{IC}⁺ and PD1_{EcTM}DAP12_{IC}⁺ NK-92 Show Increased Killing of Large PD-L1⁺ 786-O WT Tumor Spheroids

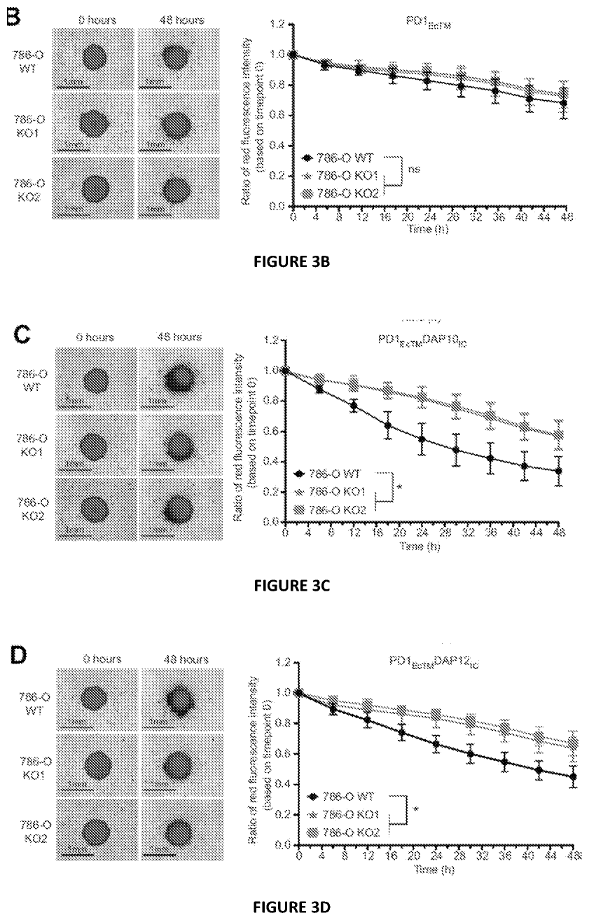

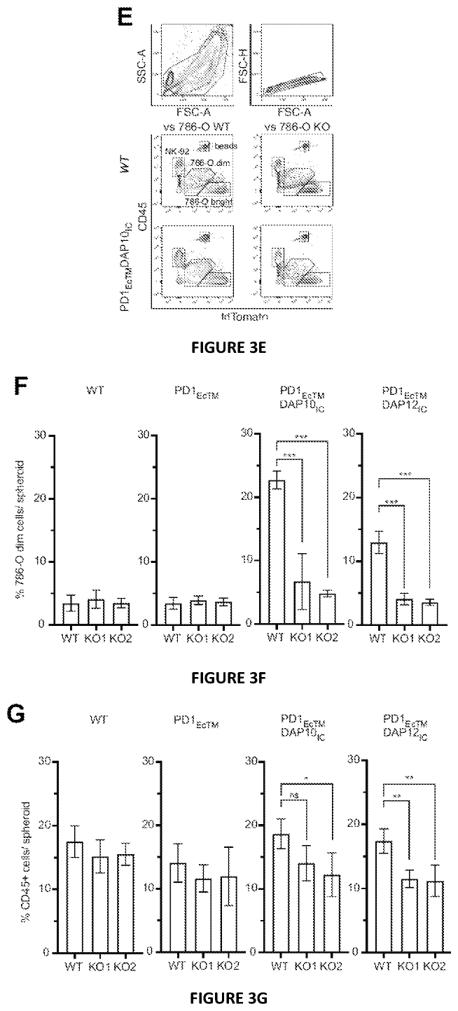

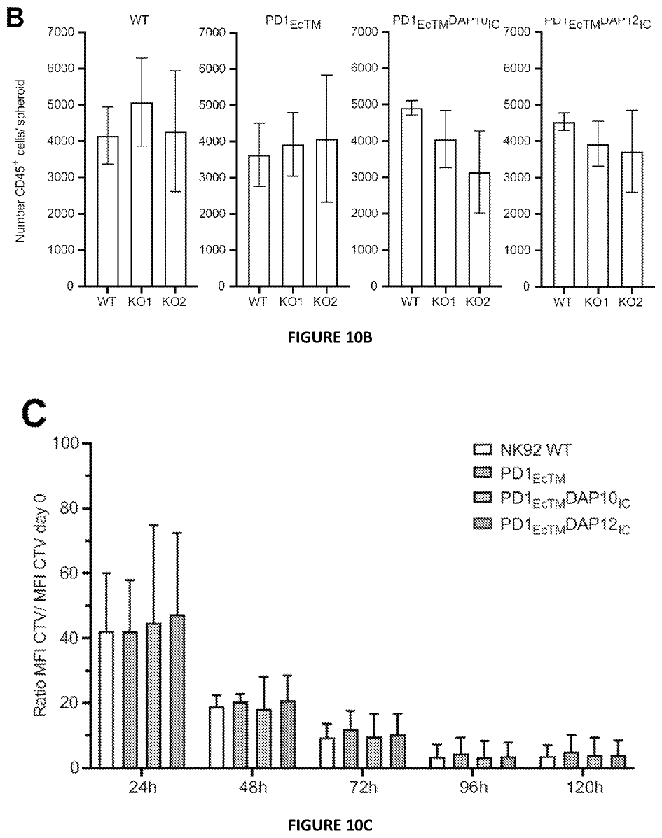

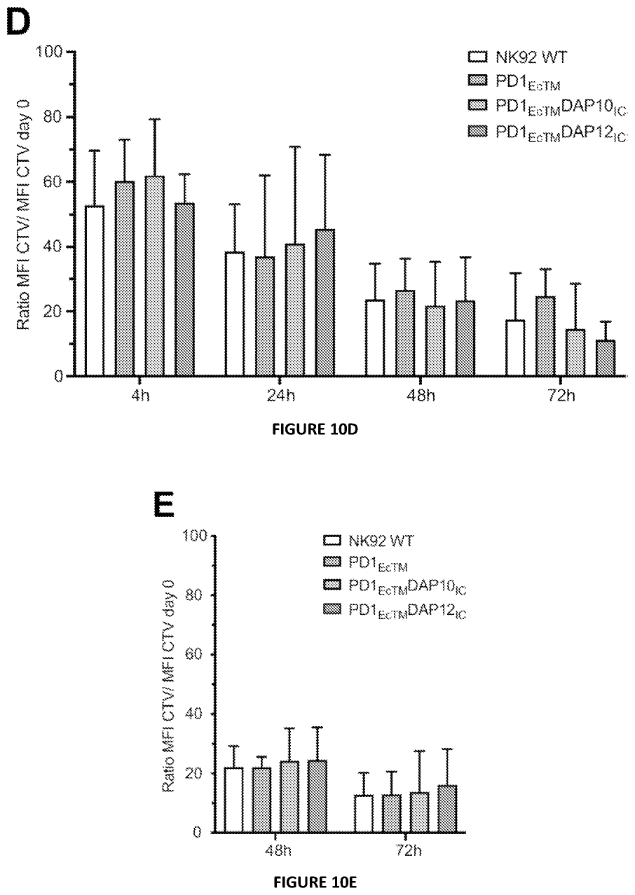

Since 786-O cells can form large tumor spheroids with diameters reaching up to one millimeter, the ability of PD1_{EcTM}DAP10_{IC}⁺ and PD1_{EcTM}DAP12_{IC}⁺ NK-92 cells to kill PD-L1⁺ 786-O WT cells was tested in the 3D co-culture model as it more accurately resembles the TME than a 2D co-culture model. For this purpose, PD-L1⁺ 786-O WT and both PD-L1⁻ 786-O KO cell lines were further transduced to express the red fluorescence protein tdTomato. Killing of 786-O spheroids was assessed based on the decrease of red fluorescence intensity as captured with the IncuCyte live cell imager (FIG. 3A-D;) and by flow cytometry (FIG. 3 E-F). FIGS. 3A-D show killing of PD-L⁺ 786-O WT versus PD-L1⁻ 786-O KO1 and PD-L1⁻ 786-O KO2 tumor spheroids by NK-92 WT, PD1_{EcTM}⁺, PD1_{EcTM}DAP10_{IC}⁺ or PD1_{EcTM}DAP12_{IC}⁺ NK-92 cells. Each data point represents the mean (+/−SD) of six independent experiments performed in duplicates. Red fluorescence intensity decreased from 100% to 70% (+/−11%) in PD-L1⁺ 786-O WT and PD-L1⁻ 786-O KO cell lines over a 48 hours period when NK-92 WT or PD1_{EcTM}⁺ NK-92 cells were added to the tumor spheroids, with no significant differences between the three target cell lines. PD1_{EcTM}DAP10_{IC}⁺ NK-92 cells resulted in a reduction of red fluorescence intensity from 100% to 35% (+/−10%) in the PD-L1⁺ 786-O WT cells and from 100% to 60% (+/−9%) in the PD-L1⁻ 786-O KO cell lines, with statistical significance between the PD-L1⁺ and PD-L1⁻ target cell lines. Similarly, there was a significant decrease in red fluorescence intensity in PD-L1⁺ 786-O WT cells (100% to 45% (+/−7%)) compared to PD-L1⁻ 786-O KO cell lines (100% to 65% (+/−8%)) when PD1_{EcTM}DAP12_{IC}⁺ NK-92 cells were added. The tumor spheroids were therafter harvested, washed and dissociated to assess spheroid killing by flow cytometry (FIG. 3 E-F; FIG. 10A). In FIGS. 3E-G, Tumor spheroids were collected, washed, dissociated and analysed by flow cytometry. Displayed is the gating strategy (E), the percentage of 786-O^{dim} cells per spheroid (F) and percentage of CD45⁺ NK-92 cells per spheroid (G). Each data point represents the mean (+/−SD) of three independent experiments performed in duplicates. Statistical significance (* p<0.05; ** p<0.01; *** p<0.001) was determined with a two-way ANOVA test. FIG. 10A shows the percentage of 786-O^{bright} cells per spheroid (A) and the total number of CD45⁺ PD1-CSR⁺ NK-92 cells per spheroid (B). Each data point represents the mean (+/−SD) of three independent experiments performed in duplicates. Statistical significance (* p<0.5) was determined with a two-way ANOVA test. Live 786-O cells showed a high expression of tdTomato and were classified as 786-O^{bright} while dying 786-O cells gradually lost tdTomato expression and were classified as 786-O^{dim} cells. As expected, both PD1_{EcTM}DAP10_{IC}⁺ and PD1_{EcTM}DAP12_{IC}⁺ NK-92 cells, but not NK-92 WT or PD1_{EcTM}⁺ NK-92 cells, led to a significant increase in the 786-O^{dim} population and corresponding decrease in the 786-O^{bright} population against the PD-L1⁺ 786-O WT but not PD-L1⁻ 786-O KO cell lines. Furthermore, the percentage and total amount of CD45⁺ cells within the 786-O tumor spheroids was assessed (FIG. 3 G, FIG. 10B). FIG. 10B shows the percentage of 786-O^{bright} cells per spheroid (A) and the total number of CD45⁺ PD1-CSR⁺ NK-92 cells per spheroid (B). Each data point represents the mean (+/−SD) of three independent experiments performed in duplicates. Statistical significance (* p<0.5) was determined with a two-way ANOVA test. There was a statistically significant difference in the percentage of CD45⁺ cells within the PD-L1⁻ 786-O WT spheroid compared to the PD-L1⁻ 786-O KO spheroids when PD1_{EcTM}DAP10_{IC}⁺ and PD1_{EcTM}DAP12_{IC}⁺ NK-92 cells were added. However, no statistical significant difference was observed for the absolute numbers of CD45⁺ cells. Lastly, proliferation of PD1-CSR⁺ NK-92 cells was measured. No significant differences in proliferation of PD1_{EC-TM}DAP10_{IC}⁺ or PD1_{EC-TM}DAP12_{IC}⁺ NK-92 cells compared to WT or PD1_{EC-TM}⁺ NK-92 cells were observed when cultured alone (FIG. 10 C), exposed one-time to PD-L1⁺ 786-O WT cells (FIG. 10 D) or repetitively exposed to PD-L1⁺ 786-O WT cells (FIG. 10 E). FIGS. 10C-E show the proliferation of PD1-CSR⁺ NK-92 cells without stimulation (C), stimulated once with PD-L1⁺ 786-O WT cells (D) or stimulated on two consecutive days with PD-L1⁺ 786-O WT cells (E). Displayed is the mean fluorescence intensity (MFI) of CTV at the indicated timepoints. Each data point represents the mean (+/−SD) of one experiment performed in duplicates.

In summary, both PD1_{Ec-TM}DAP10_{IC}⁺ and PD1_{Ec-TM}DAP12_{IC}⁺ NK-92 cells increased killing of large PD-L1⁺ 786-O WT tumor spheroids over a 48 hours period.

PD1_{EcTM}DAP10_{IC}⁺ and PD1_{EcTM}DAP12_{IC}⁺ pNK Cells Increase Degranulation and Cytokine Expression Against PD-L1⁺ Raji Cells

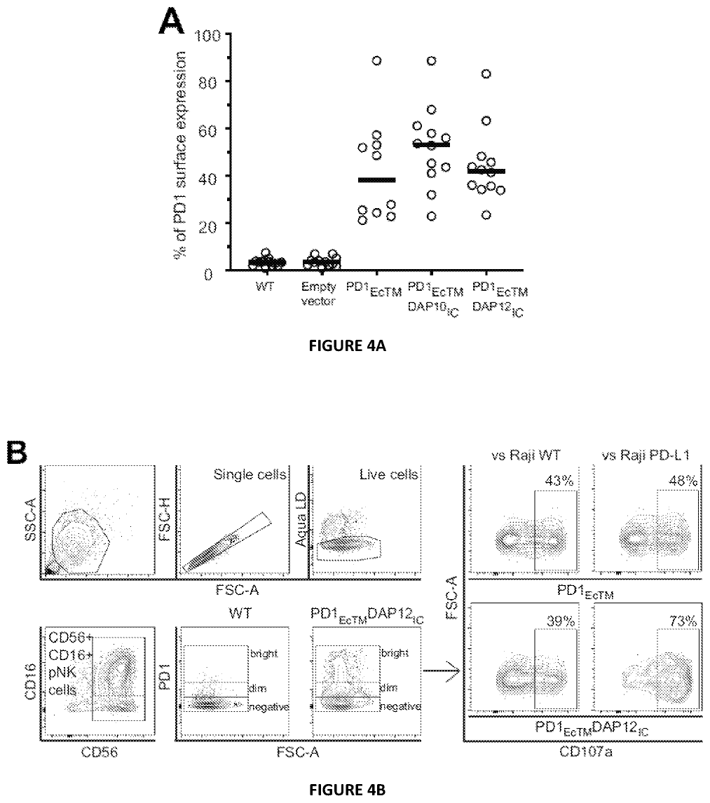

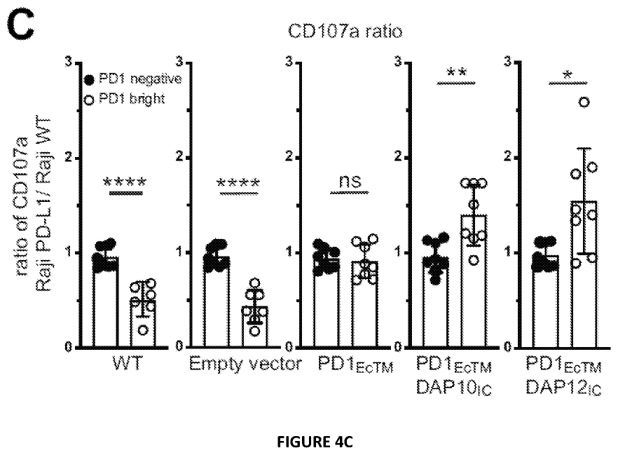

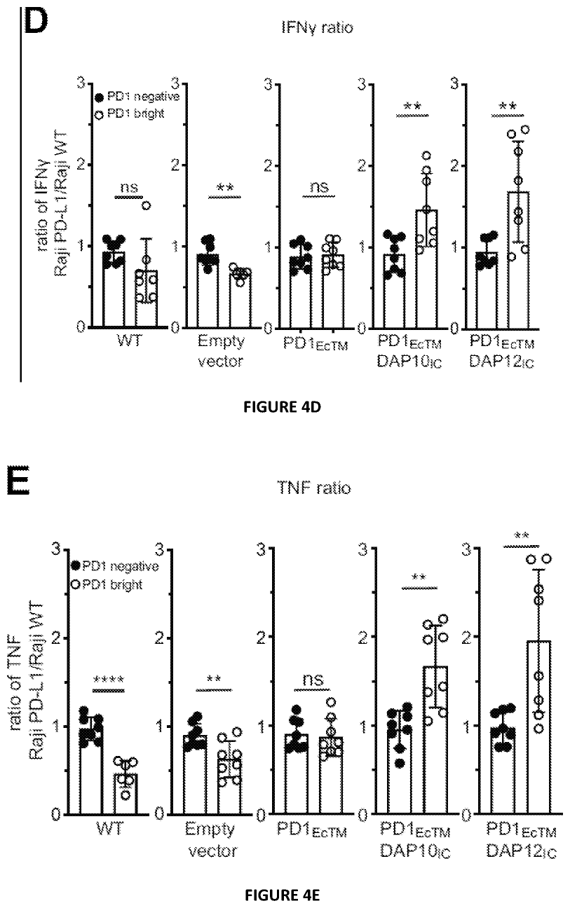

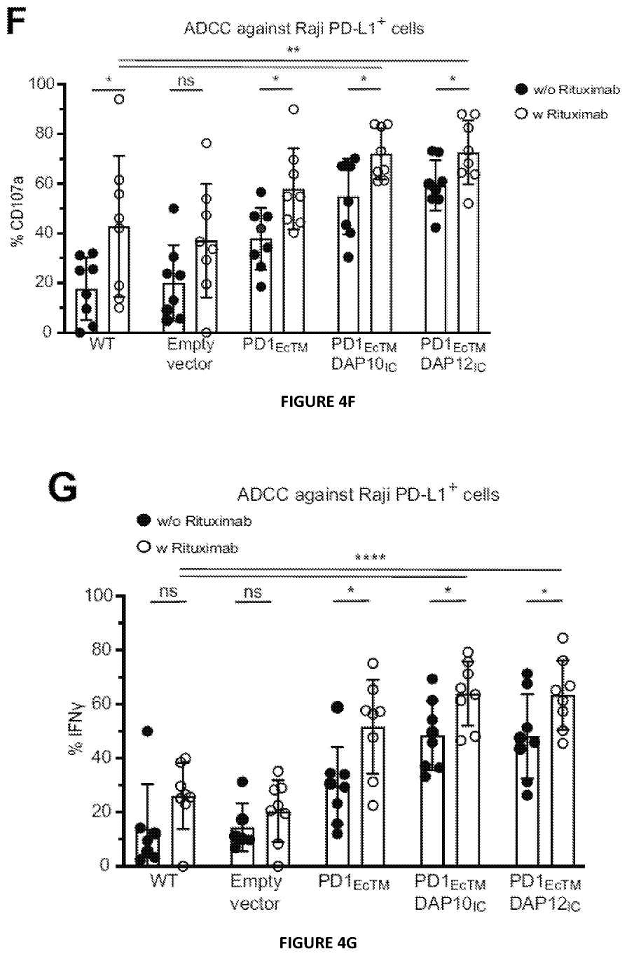

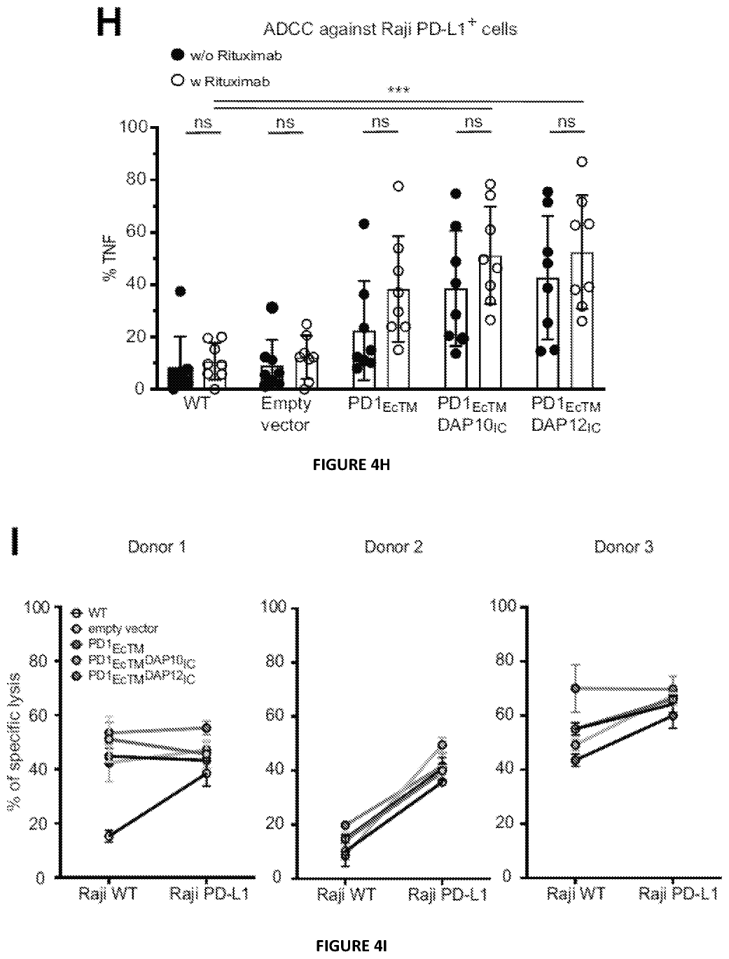

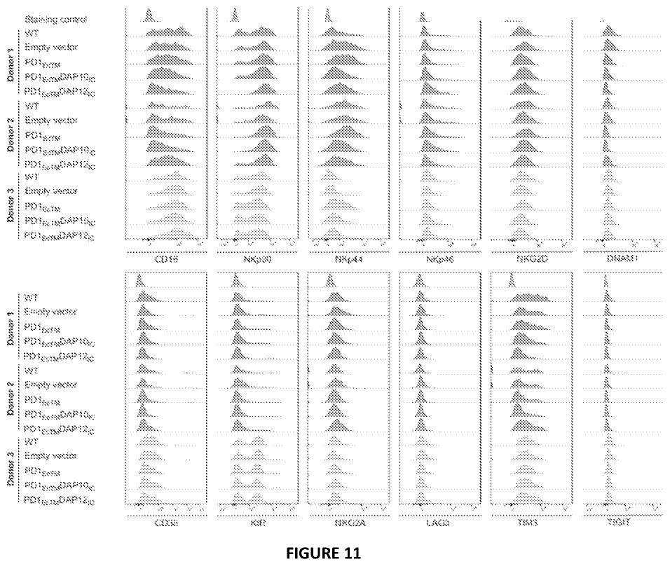

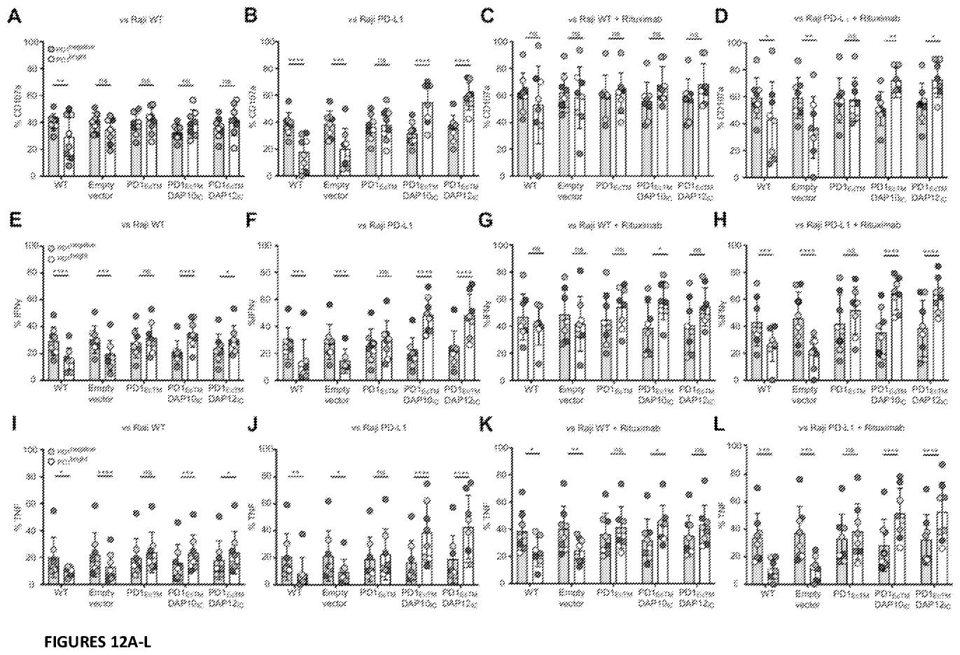

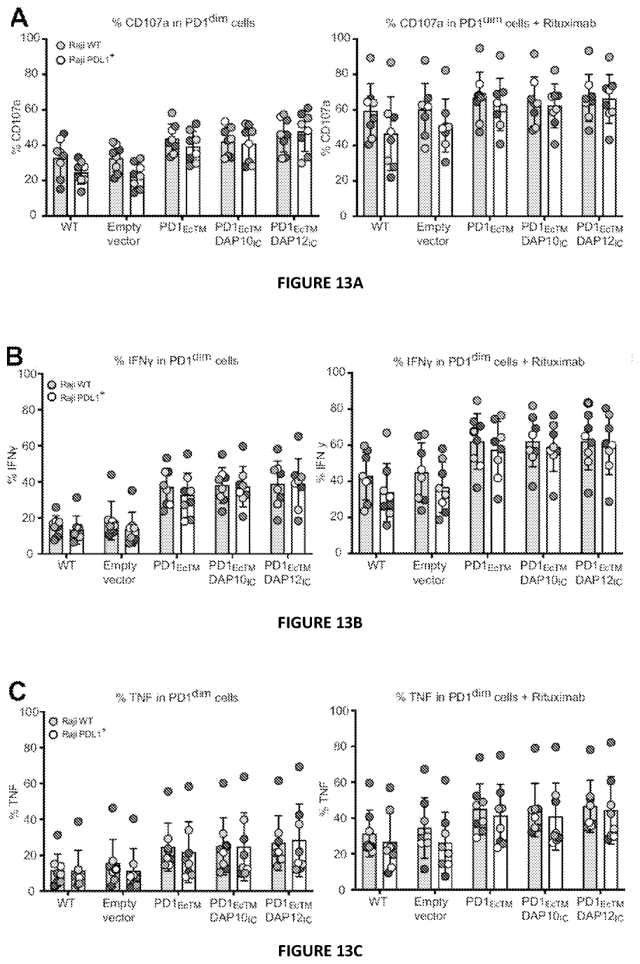

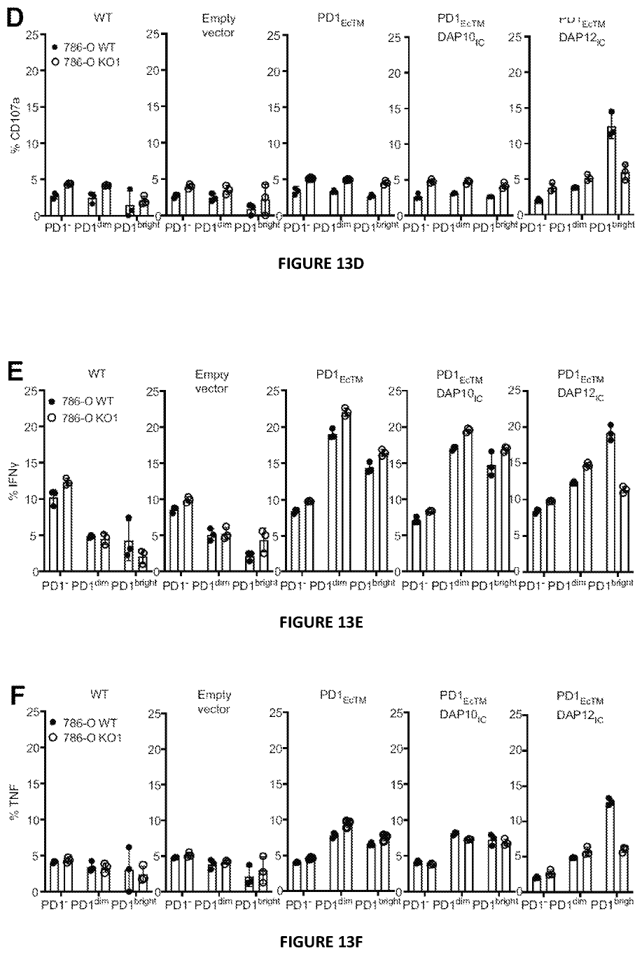

With the aim to implement PD1-CSR for adoptive cell therapies, the function of PD1_{EcTM}DAP10_{IC} and PD1_{EcTM}DAP12_{IC} constructs in pNK cells, isolated from healthy donor PBMCs, was tested. While PD1 surface expression in untransduced pNK cells or mock-transduced (empty vector) pNK cells remained below 5%, its expression increased on average to 42% (+/−22%), 52% (+/−17%) and 45% (+/−16%) in the pNK cells transduced with either PD1_{EcTM}, PD1_{EcTM}DAP10_{IC} or PD1_{EcTM}DAP12_{IC} CSR, respectively (FIG. 4A). FIG. 4A shows PD1 surface expression on untransduced or transduced CD56⁺ CD3⁻ pNK cells. Each dot represents PD1 surface expression on one individual donor (n=12). The mean of all 12 donors is displayed. Two separate levels of PD1 surface expression were observed and CD56⁺ CD16⁺ pNK cells were classified as either PD1^{dim} or PD 1^{bright} (FIG. 4 B). FIG. 4B shows a gating strategy for degranulation assays. WT and empty vector transduced cells were gated on PD 1^{dim} population while PD1-CSR⁺ cells were gated on the PD 1^{high} population. The expression of the main activating and inhibitory NK cell receptors on PD1⁺ PD1-CSR⁺ pNK cells compared to PD1⁺ WT or mock-transduced pNK cells from three different donors was measured by flow cytometry (FIG. 11). FIG. 11 shows flow ctometry plots depict the expression activating and inhibitory NK cell receptors on WT or PD1-CSR+ pNK cells from three different donors. Although inter-individual differences in receptor expression were observed, the genetic modification of pNK cells with PD1-CSR did not cause any consistent intra-individual phenotypic changes of pNK cells. CD107a, IFNγ and TNF expression were measured in a degranulation assay against PD-L1⁺ Raji cells and PD-L1⁻ Raji WT cells (FIG. 4 C-E, FIG. 12A-L, FIG. 13A-C). FIGS. 12A-L show the percentage of CD107a (A-D), IFNγ (E-H) or TNF (I-L) against PD-L1⁺ Raji cells or PD-L1⁻ Raji WT cells by PD1^{dim} WT or mock-transduced pNK cells or PD 1^{high} PD1-CSR⁺ pNK cells vs PD 1^{negative} pNK cells with or without the addition of Rituximab. Each dot represents the mean of one donor performed in duplicates or triplicates (n=8). Colour code refers to the same donor. The mean+/−SD of all 8 donors is displayed. Statistical significance (* p<0.05; ** p<0.01) was determined with a two-way ANOVA test. FIGS. 13A-C show the percentage of CD107a (A), IFNγ (B) or TNF (C) against PD-L1⁺ Raji cells or PD-L1⁻ Raji WT cells by PD1^{dim} WT or mock-transduced pNK cells or PD 1^{dim} PD1-CSR⁺ pNK cells vs PD 1^{negative} pNK cells with or without the addition of Rituximab. Each dot represents the mean of one donor performed in duplicates or triplicates (n=8). Colour code refers to the same donor. The mean+/−SD of all 8 donors is displayed. To facilitate direct comparison, the fold ratio of CD107a, IFNγ and TNF expression against PD-L1⁺ Raji cells compared to PD-L1⁻ Raji WT cells is displayed, with numbers below one indicating a reduction and numbers above one an increase upon PD-L1 engagement in the respective parameter (FIGS. 4C-E). FIGS. 4C-E show the Ratio of CD107a (C), IFNγ (D) and TNF (E) expression by different PD-CSR⁺ pNK cells against PD-L1⁺ Raji cells vs PD-L1⁻ Raji WT cells. Each dot represents the mean of one individual donor, performed in duplicates (n=8) The mean+/−SD of all 8 donors is displayed. PD 1^{dim} pNK cells from WT or mock-transduced pNK cells showed a lower CD107a expression against PD-L1⁺ Raji cells, significantly reducing the ratio below one. On the contrary, PD1^{bright} PD1_{EcTM}⁺ pNK cells increased degranulation against PD-L1⁺ Raji cells compared to WT or mock-transduced pNK cells, raising the ratio to one. PD1^{bright} PD1_{EcTM}DAP10_{IC}⁺ and PD1^{bright} PD1_{EcTM}DAP12_{IC}⁺ pNK cells significantly increased CD107a expression against PD-L1⁺ Raji cells compared to PD-L1⁻ Raji WT cells, raising the ratio to 1.5. Similarly, the ratio of IFNγ and TNF expression against PD-L1⁺ Raji cells compared to PD-L1⁻ Raji WT cells was higher than one for the PD1^{bright} PD1_{EcTM}DAP10_{IC}⁺ and PD1^{bright} PD1_{EcTM}DAP12_{IC}⁺ pNK cells but not PD1^{bright} PD1_{EcTM}⁺ pNK cells or PD1^{dim} WT or PD1^{dim} mock-transduced pNK cells. In conclusion, PD1_{EcTM}⁺ pNK cells blocked native PD1-PD-L1 mediated pNK cell inhibition, while both PD1_{EcTM}DAP10_{IC}⁺ and PD1_{EcTM}DAP12_{IC}⁺ pNK cells reverted inhibition into an increased degranulation and cytokine expression against PD-L1⁺ Raji cells.

PD1_{EcTM}DAP10_{IC}⁺ and PD1_{EcTM}DAP12_{IC}⁺ pNK Cells Increase Degranulation and Cytokine Expression Against PD-L1⁺ Raji Cells Together with ADCC

After demonstrating functionality of PD1_{EcTM}DAP10_{IC} and PD1_{EcTM}DAP12_{IC} CSR in pNK cells, their ability to synergize with CD16 mediated ADCC was tested to evaluate their potential in combinatorial treatment approaches. The percentages of CD107a, IFNγ and TNF expression against PD-L1⁺ Raji cells and PD-L1⁻ Raji WT cells with or without the addition of the anti-CD20 mAb Rituximab were measured (FIG. 4 F-H, FIG. 12). FIGS. 4F-H show the Percentage of CD107a (F), IFNγ (G) and TNF (H) expression by different PD-CSR⁺ pNK cells against PD-L1⁺ Raji cells. Each dot represents the mean of one individual donor, performed in duplicates (n=8) The mean+/−SD of all 8 donors is displayed. Rituximab increased CD107a, IFNγ and TNF expression against PD-L1⁺ Raji cells in both PD1^{dim} WT and mock-transduced pNK cells compared to no ADCC. Likewise, PD1^{bright} PD_{EcTM}, PD1_{EcTM}DAP10_{IC}⁺ and PD1_{EcTM}DAP12_{IC}⁺ pNK cells showed increased CD107a, IFNγ and TNF expression with the addition of Rituximab compared to no ADCC. Overall, PD1^{bright} PD1_{EcTM}DAP10_{IC}⁺ and PD1_{EcTM}DAP12_{IC}⁺ pNK showed the highest increase in CD107a, IFNγ and TNF expression with values reaching up to 72% (+/−12%), 63% (+/−12%) and 51% (+/−19%), respectively, compared to WT or mock-transduced pNK cells with values approaching 42% (+/−28), 26% (+/−12) and 11% (+/−8%), respectively. Compared to PD1^{bright} PD1-CSR⁺ pNK cells, PD1^{dim} PD1_{EcTM}DAP10_{IC}⁺ and PD1_{EcTM}DAP12_{IC}⁺ pNK cells did not increase degranulation and cytokine production against PD-L1⁺ Raji cells but equalized it to the CD107a, IFNγ and TNF expression levels against PD-L1⁻ Raji cells (FIGS. 13A-C). In conclusion, both PD1_{EcTM}DAP10_{IC}⁺ and PD1_{EcTM}DAP12_{IC}⁺ pNK cells increased degranulation and cytokine expression against PD-L1⁺ Raji cells with or without the addition of Rituximab and thus reverted native PD1 mediated NK cell inhibition.

PD1_{EcTM}DAP12_{IC}⁺ pNK Cells Increase Degranulation and Cytokine Expression Against PD-L1⁺ 786-O WT Cells

To extend these results, degranulation and cytokine expression of PD1-CSR⁺ pNK cells was also measured against PD-L1⁺ 786-O WT and PD-L1⁻ 786-O KO cells (FIGS. 13D-F). FIGS. 13D-F show the percentage of CD107a (D), IFNγ (E) or TNF (F) against PD-L1⁺ 786-O WT cells or PD-L1⁻ 786-O KO1 cells by different PD1^{negative} vs PD 1^{dim} vs PD 1^{high} PD1-CSR⁺ pNK cells. Experiment was performed with one donor in triplicates. Statistical significance (* p<0.05; ** p<0.01) was determined with a two-way ANOVA test.

Similar to the data from NK92 cell lines, WT or mock-transduced pNK cells showed a low expression of CD107a, IFNγ and TNF against both PD-L1⁺ 786-O WT and PD-L1⁻ 786-O KO cells, with no significant differences between the target cell lines. Compared to that PD1^{bright} PD1_{EcTM}DAP12_{IC}⁺ pNK cells, but not PD1^{dim} PD1_{EcTM}DAP12_{IC}⁺ pNK cells, significantly increased CD107a, IFNγ and TNF expression against PD-L1⁻ 786-O WT cells compared to PD-L1⁻ 786-O KO cells. While PD1_{EcTM}⁺ and PD1_{EcTM}DAP10_{IC}⁺ pNK cells did not correlate with a higher CD107a expression, they showed a higher IFNγ and TNF expression against both PD-L1⁺ 786-O WT and PD-L1⁻ 786-O KO cells. All in all, PD1^{bright} PD1_{EcTM}DAP 12_{IC}⁺ pNK cells increased degranulation and cytokine expression against PD-L1⁺ 786-O WT cells.

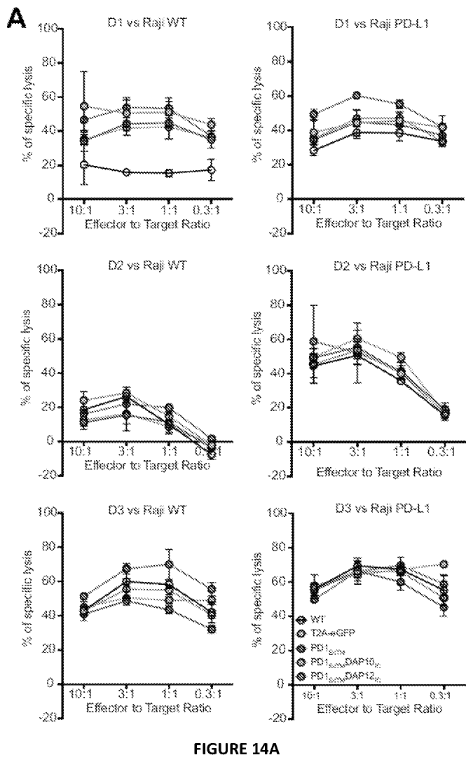

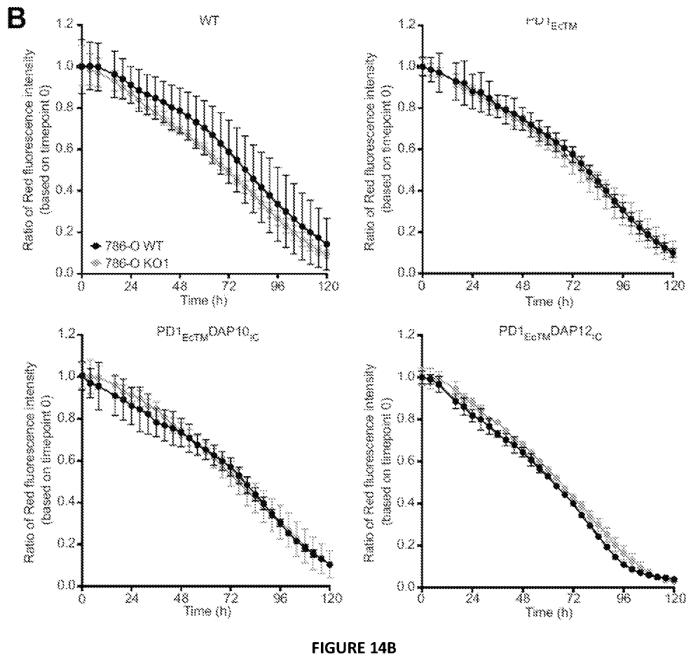

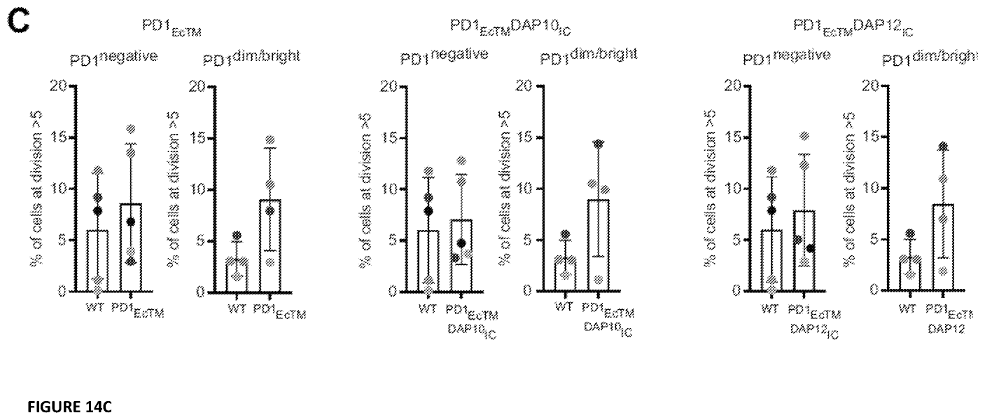

PD1_{EcTM}DAP10_{IC}⁺ and PD1_{EcTM}DAP12_{IC}⁺ pNK Cells do not Alter Killing of PD-L1⁺ Target Cells
A) Next, the ability of the PD1-CSR enriched pNK cells to kill PD-L1⁺ and PD-L1⁻ target cells in a 2D and 3D co-culture model was assessed. No difference in killing of PD-L1⁺ Raji cells compared to PD-L1⁻ Raji WT cells by PD1_{EcTM}DAP10_{IC}⁺ or PD1_{EcTM}DAP12_{IC}⁺ pNK cells from three different donors compared to WT, mock-transduced or PD1_{EcTM}⁺ pNK cells was observed (FIG. 41, FIG. 14A). FIG. 4I shows the killing of PD-L1⁺ Raji cells versus PD-L1⁻ Raji WT cells by different PD-CSR⁺ pNK cells at an E:T of 1:1. Displayed are data from 3 independent donors with each data point representing the mean (+/−SD) of one experiment performed in triplicates. Statistical significance was determined with a Students t test (* p<0.05, ** p<0.01, *** p<0.001, **** p<0.0001). FIG. 14A shows killing of PD-L1⁺ Raji cells versus PD-L1⁻ Raji WT cells by different PD1-CSR⁺ pNK cells. Displayed are data from 3 independent donors with each data point representing the mean (+/−SD) of one experiment performed in triplicates. Similarly, neither PD1_{EcTM}DAP10_{IC}⁺ nor PD1_{EcTM}DAP12_{IC}⁺ pNK cells increased killing of PD-L1⁺ 786-O WT tumor spheroids compared to PD-L1⁻ 786-O KO tumor spheroids (FIG. 14 B). FIG. 14B shows the killing of PD-L1⁺ 786-O WT versus PD-L1⁻ 786-O KO1 tumor spheroids by different PD1-CSR⁺ pNK cells. Displayed are data from one donor, representative of a total of four donors assessed. Each data point represents the mean (+/−SD) of one experiment performed in triplicates. However, this lack of killing ability might be due to the fact that pNK cells were not sorted for high expression of PD1-CSR prior to use. Finally, the proliferative capacity of PD1-CSR⁺ pNK cells was measured (FIG. 14C). FIG. 14C shows the proliferation of PD1^{dim} WT pNK cells or different PD1^{bright} PD1⁺ CSR-pNK cells versus PD1^{negative} pNK cells in co-culture with PD-L1⁺ Raji cells. Each dot represents the mean of one donor performed in triplicates (n=4). The mean+/−SD of all 4 donors is displayed. Statistical significance (* p<0.05; ** p<0.01) was determined with a two-way ANOVA test. PD 1^{dim} WT pNK cells showed a lower proliferation rate compared to PD1^{negative} WT pNK cells. In contrast, PD1^{bright} PD1-CSR⁺ pNK cells increased the proliferation rate above the value observed in PD1^{dim} WT pNK cells closer to the value observed in PD1^{negative} pNK cells. All in all, pNK cells, enriched with PD1_{EcTM}DAP10_{IC}⁺ and PD1_{EcTM}DAP12_{IC}⁺ pNK cells, did not show an increased killing of PD-L1⁺ tumor target cells.
PD1_{EcTM}DAP10_{IC}⁺ and PD1_{EcTM}DAP12_{IC}⁺ pNK Cells from Patients with Newly Diagnosed MM Increase Degranulation and Cytokine Production Against Autologous PD-L1⁺ Tumor Samples

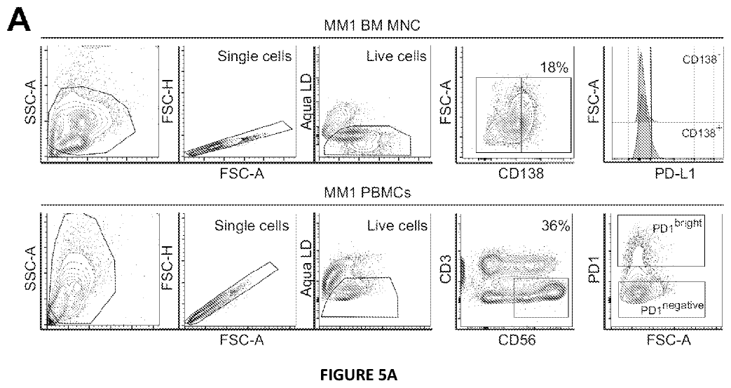

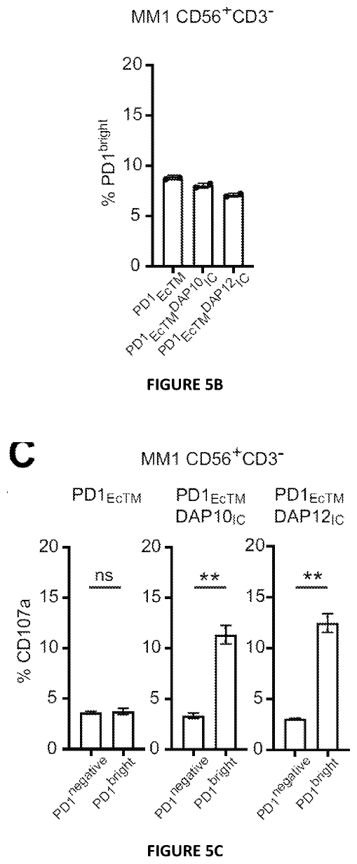

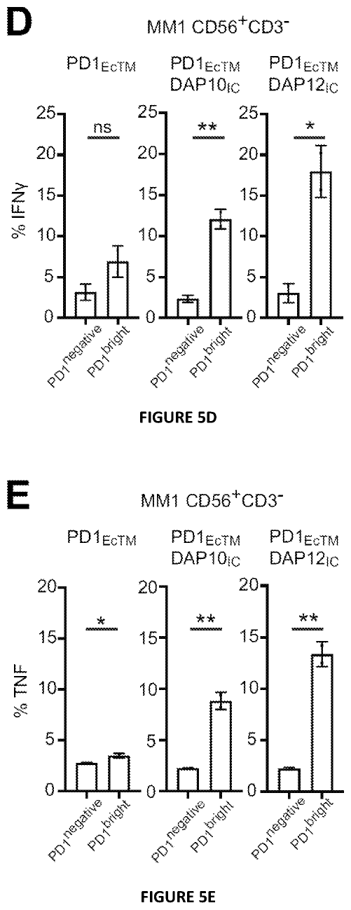

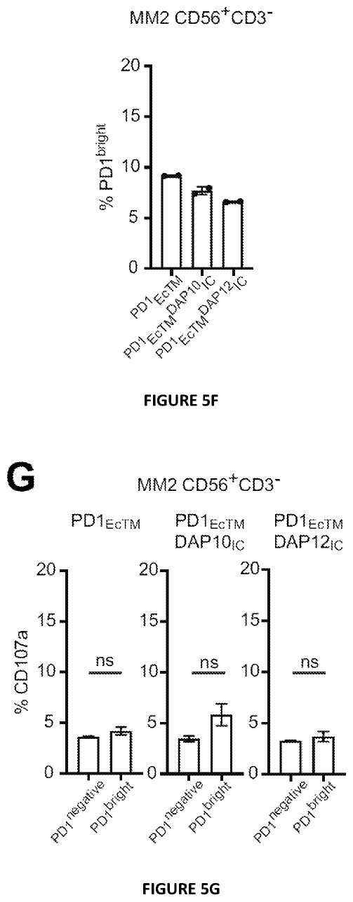

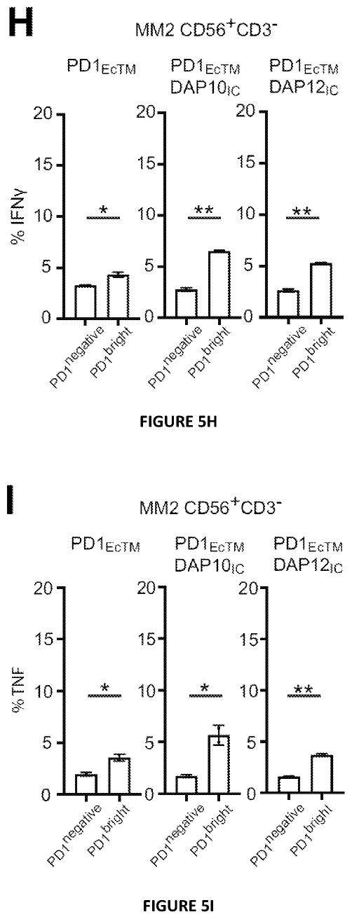

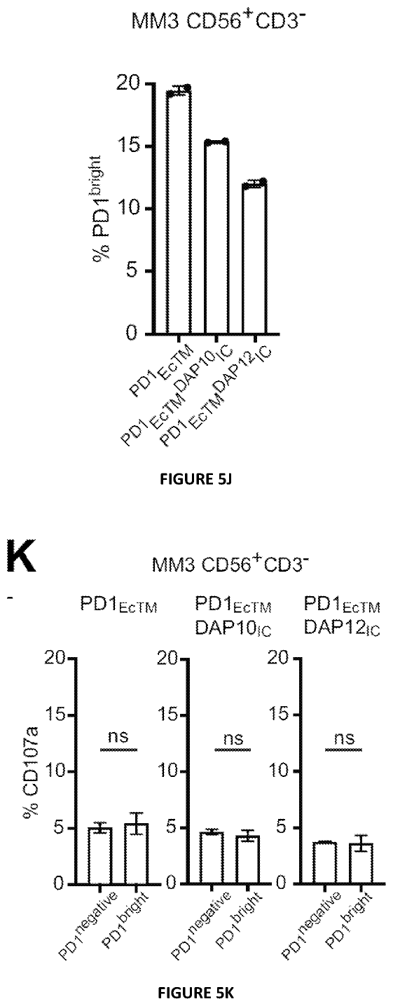

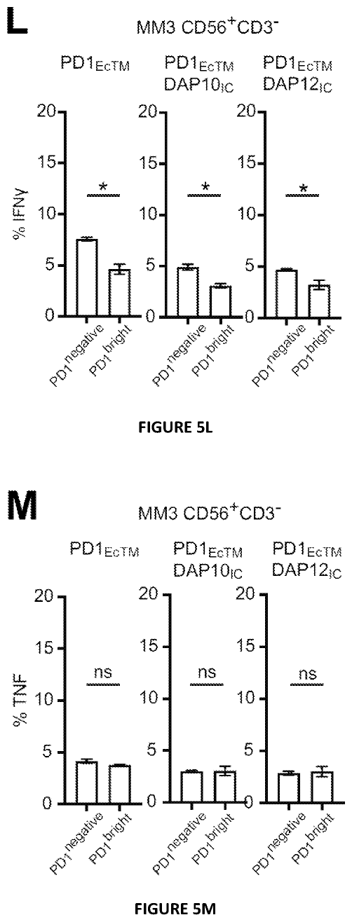

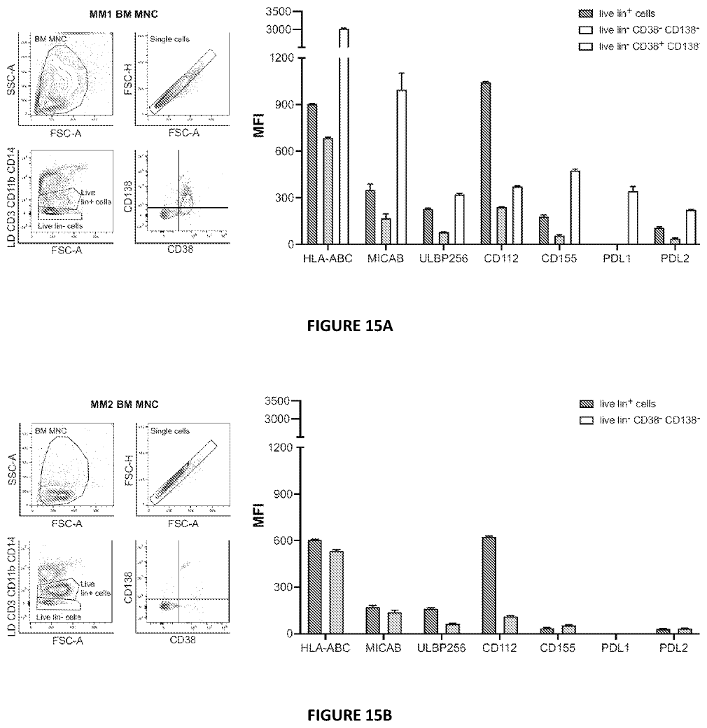

After establishing that both PD1_{EcTM}DAP10_{IC}⁺ and PD1_{EcTM}DAP12_{IC}⁺ pNK cells from healthy donors increased degranulation and cytokine expression against PD-L1⁺ tumor cell lines, their function in pNK cells from patients with MM against autologous bone marrow mononuclear cells (BM MNC) was evaluated. For this, PBMCs from three patients with newly diagnosed MM were expanded for 13 days prior to transduction with lentiviral vectors, encoding PD1_{EcTM}, PD1_{EcTM}DAP10_{IC} and PD1_{EcTM}DAP12_{IC} CSR. Degranulation was performed on day 4 after transduction with approximately 40% CD56⁺CD3⁻ pNK cells among the expanded PBMCs (FIG. 5A). FIG. 5A contains flow cytometry plots show the gating strategy of BM MNC (upper panel) and PBMCs (lower panel) from newly diagnosed MM patients. CD138 expression on BM MNC, indicative of malignant plasma cells, was measured by flow cytometry and only detected in BM MNC from donor 1 (MM1 BM MNC), but not donor 2 (MM2 BM MNC) or donor 3 (MM3 BM MNC). A more detailed phenotypic analysis of BM MNC from donor 1 and donor 2 is provided in figure S10. PD-L1 and PD-L2 expression was detected on CD138⁺ cells, but only at very low levels on CD138⁻ BM MNCs (FIG. 5A, FIGS. 15A-B). FIGS. 15A-B show that specifically, BM MNC were gated on single cells and thereafter live lineage⁺ (lin⁺) cells or live lineage⁻ (lin⁻) cells. Lin⁺ cells are defined as CD3⁺ CD11b⁺ CD14⁺ cells. The live lin⁻ cells were further classified based on the expression of CD38 and CD138 cells to be able to distinguish double positive malignant plasma cells. Thus, the expression of NK cell ligands was analysed on three different cell populations, namely live lin⁺ cells versus live lin⁻ CD38⁻CD138⁻ cells versus live lin⁻ CD38⁺CD138⁺ cells, for donor 1. Since no malignant plasma cells could be detected in the BM MNC from donor 2 only two cell populations, namely live lin⁺ cells versus live lin⁻ CD38⁻CD138⁻ cells were further analysed. The histograms depict the MFI of different NK cell ligands on the above described cell populations. The percentage of PD1^{bright} cells on CD56⁺CD3⁻ pNK cells ranged between 7% to 20% depending on the CSR construct and donor (FIG. 5 B, F, J). FIG. 5 B shows the Percentage of PD1^{bright} cells among CD56⁺CD3⁻ pNK cells from MM donor 1. FIG. 5F shows the percentage of PD1^{bright} cells among CD56⁺CD3⁻ pNK cells from MM donor 2 FIG. 5J shows the percentage of PD1^{bright} cells among CD56⁺CD3⁻ pNK cells from MM donor 3 Both PD 1 bright PD1_{EcTM}DAP10_{IC}⁺ and PD1_{EcTM}DAP12_{IC}⁺ pNK cells from donor 1 increased CD107a, IFNγ and TNF expression significantly against autologous BM MNC two- to three fold compared to PD1^{negative} pNK cells (FIG. 5 C-E). FIG. 5 C-E show the percentage of CD107a (C), IFNγ (D) and TNF (E) by different PD1^{negative} vs PD1^{bright} PD1-CSR⁺ pNK cells from donor 1 against autologous BM MNC. PD1^{bright} PD1_{EcTM}DAP10_{IC}⁺ and PD1_{EcTM}DAP12_{IC}⁺ pNK cells from donor 2 did not increase CD107a expression, but showed an increased IFNγ and TNF expression compared to PD1^{negative} pNK cells (FIG. 5 G-I). FIG. 5 G-I show the percentage of CD107a (G), IFNγ (H) and TNF (I) by different PD 1^{negative} vs PD1^{bright} PD-CSR⁺ pNK cells from donor 2 against autologous BM MNC. PD1^{bright} PD1_{EcTM}DAP10_{IC}⁺ and PD1_{EcTM}DAP12_{IC}⁺ pNK cells from donor 3 did not increase CD107a, IFNγ or TNF expression, but showed a slight decreased IFNγ response (FIG. 5 K-M). FIG. 5 K-M show the percentage of CD107a (K), IFNγ (L) and TNF (M) by different PD1^{negative} vs PD 1^{bright} PD-CSR⁺ pNK cells from donor 3 against autologous BM MNC. Displayed are data from each donor with each data point representing the mean (+/−SD) of one experiment performed in duplicates. Statistical significance was determined with a Students t test (* p<0.05, ** p<0.01). These data confirm that PD1_{EcTM}DAP10_{IC}⁺ and PD1_{EcTM}DAP12_{IC}⁺ pNK augment degranulation and cytokine expression against autologous CD138⁺ PD-L1⁺ malignant bone marrow cells.

### DISCUSSION

In this paper, we demonstrate that PD1-based CSR revert NK cell inhibition imposed by PD1-PD-L1 engagement and, hence, are able to skew the response towards NK cell activation. The results emphasize that replacement of the ITIM and ITSM domain of PD1 by either an ITAM or YINM motif confers a higher degranulation and cytokine production by both NK-92 and pNK cells towards PD-L1 expressing target cells in 2D and 3D tumor co-culture models. Most importantly, pNK cells from patients with MM were successfully transduced to express PD1_{EcTM}DAP10_{IC} or PD1_{EcTM}DAP12_{IC} CSR and showed higher degranulation and cytokine expression against autologous CD138⁺ PD-L1⁺ tumor samples.

Different to CAR, the present CSR shall enhance NK cell cytotoxicity in concert with general target cell recognition and tip the balance towards activation in an immunosuppressive TME. The aim in the present study was to design CSR that are not activating NK cells towards healthy tissue where PD1 ligands are abundantly expressed and can explain common side-effects of immune-checkpoint blockade with mAb [26]. Therefore, the human canonical sequence of PD1 without any further modification was employed. Furthermore, only one signaling domain was used compared to the second and third generation CARs that are designed with different costimulatory domains. Whether PD-L1 targeting CAR NK cells would, however, cause severe side-effects is yet not elucidated. PD-L1 targeting high-affinity NK-92 cells (PD-L1-t-haNK) showed promising preclinical results and are currently in early phase clinical trials (NCT04050709, NCT04847466, NCT04927884) [27]. The results and toxicity profile of these PD-L1 CAR expressing NK-92 cells are eagerly awaited. However, employing the extracellular domain of PD1, instead of the single-chain fragment targeting PD-L1, poses the advantage of recognizing both PD-L1 and PD-L2. In humans, PD-L2 is mainly expressed on professional antigen-presenting cells and over-expressed in cancer cells as well as stromal and epithelial cells of several tumor types [28]. Targeting both PD1 ligands was associated with a better clinical outcome in lung cancer [29]. Besides PD1, NK cells express a plethora of canonical checkpoints that are important both for control of activation as well as retention of educated state upon adoptive transfer [30, 31]. The surface retained checkpoints include NKG2A, T cell immunoreceptor with Ig and ITIM domains (TIGIT), Lymphocyte Activating Gene 3 (LAG3), T cell immunoglobulin domain and mucin domain 3 (TIM3) as well as inhibitory KIRs. It is conceivable that some of these receptors could also be engineered in a similar fashion as described here for PD1. PD1-CSR⁺ pNK cells from MM patient number 3 showed a decrease in IFNγ production against autologous BM MNCs. Unfortunately, we were not able to determine the factors leading to this small but significant reduction in cytokine expression. Taken the complexity of the immunosuppressive TME into account, it is conceivable that PD1-CSR expressing NK cells might be inhibited by other soluble or receptor-mediated factors. A solution could be the combination of PD1-CSR⁺ pNK cells with other immune checkpoint targeting therapies. Monalizumab, a monoclonal antibody against NKG2A that is widely used in clinical trials, promoted both NK and CD8⁺ T cell anti-cancer functions, especially in combination with PD1-PD-L1 blockade [30]. In line with this, disruption of NKG2A in primary NK cells improved NK cell cytotoxicity against primary MM cells [32].

Blockade of immune checkpoint receptors such as PD1 or TIGIT with mAb was shown to restore NK cell effector functions against tumor cells [15, 33]. However, the majority of available antibodies merely blocks the PD1-PD-L1 interaction and does not induce ADCC to enhance NK cell functions. So far, avelumab is the only PD-L1-targeting antibody available with ADCC function [34]. To date, no clinical study evaluated the combination of avelumab with adoptive NK cell therapy. Here, we show that both PD1_{EcTM}DAP10_{IC} and PD1_{EcTM}DAP12_{IC} revert PD1 based NK cell inhibition, with PD1_{EcTM}DAP12_{IC}⁺ cells eliciting a higher increase. In line with our present findings, a PD1-NKG2D CSR with 4-1BB costimulatory domain enhanced killing of PD-L1⁺ target cells, but did not increase cytokine release [35]. NKG2D is a type-II transmembrane protein that dimerizes and forms a hexameric structure with four DAP10 molecules [36]. Moreover, a DAP12 based CAR increased both target cell killing and IFNγ production by pNK cells [37]. The effector cell functionality of CAR-DAP12 transduced NK cells was higher than CAR-CD3ζ transduced cells. Both PD1-CSR constructs were able to revert NK cell hypofunctionality induced by native PD1-PD-L1 signaling. However, this increase was only observed in transduced PD1^{bright} pNK cells. In contrast, PD1^{dim} cells blocked native PD1-PD-L1 engagement and restored degranulation and cytokine secretion. With 5-10% of PD1^{bright} cells within the NK cell product, we have not observed an overall higher target cell killing.

The present findings indicate that PD1-CSR⁺ pNK cells could be employed in combinatorial treatment approaches such as in combination with mAb. Therefore, the ability of PD1-CSR⁺ pNK to engage in ADCC was studied and demonstrated that both PD1_{EcTM}DAP10_{IC}⁺ and PD1_{EcTM}DAP12_{IC}⁺ synergistically increased degranulation against PD-L1⁺ Raji cells in combination with Rituximab. Another mAb that is known to work mainly via ADCC is Daratumumab that targets CD38 expressed on malignant plasma cells [38]. Daratumumab is approved as a frontline therapy in patients with newly diagnosed MM [39]. Furthermore, NK cell based therapies are currently in early-phase clinical trials for MM (NCT04558853, EudraCT: 2020-000994-26) [40, 41]. The results of these trials are eagerly awaited. We envision an indication for PD1-CSR⁺ pNK cells in patients with MM, a disease in which immune checkpoint blockade with mAb has failed. Monotherapy with the monoclonal PD1 antibody nivolumab in heavily pretreated MM patients only led to a stable disease without significant disease regression [42]. Two phase III clinical trials, studying the combinatorial application of PD1 receptor blockade by pembrolizumab with an immunomodulatory drug (IMiD) and dexamethasone (Keynote-183, Keynote-185), had to be suspended in 2017 due to dissatisfactory interim results, revealing increased death rates among patients that were enrolled in the experimental arm [43, 44]. Specifically, severe cardiac events, myocarditis and pneumonia were higher in the group that received pembrolizumab, causing increased death rates. Studies are ongoing to determine patient cohorts, combination regimens and treatment agents to efficiently target the PD1-PD-L1 axis in MM and improve patient outcome. Recently, avelumab showed a good toxicity profile but unfortunately no clinical benefit in combination with radiotherapy for relapsed or refractory MM [45]. Promisingly, our preclinical data show that both PD1_{EcTM}DAP10_{IC}⁺ and PD1_{EcTM}DAP12_{IC}⁺ pNK cells from newly diagnosed MM patients increase degranulation and cytokine production against autologous PD-L1⁺ CD138⁺ BM MNC while sparing PD-L1⁻ CD138⁻ samples. However, PD1-CSR⁺ pNK cells could potentially also target PD-L1 expressed on other cells of the TME such as myeloid-derived suppressor cells (MDSC) or tumor-associated macrophages (TAM) and thus re-shape the TME via increased cytokine expression or reduction of pro-tumorigenic cell numbers. Further studies to advance PD1-CSR⁺ pNK cells for the treatment of MM; e.g., in combination with Daratumumab, are warranted. Specifically, PD1-CSR should be tested in pNK cells from a larger cohort of patients with MM to confirm our observations reported here.

In conclusion, we have here demonstrated that PD1_{EcTM}DAP10_{IC}⁺ and PD1_{EcTM}DAP12_{IC}⁺ CSR revert PD1-PD-L1 induced NK cell inhibition. PD1-CSR⁺ NK cells hence represent a feasible approach for future adoptive NK cell-based immunotherapy platforms in human cancer treatment.

SEQ ID NOS

| | |
|---|---|
| SEQ ID NO: 1 | PD1_{EcTM} SEQ ID NO 1 |
| SEQ ID NO: 2 | AA 1-170 of PD1 SEQ ID NO 2 |
| SEQ ID NO: 3 | A long hinge region SEQ ID NO 3 |
| SEQ ID NO: 4 | AA sequence 153-220 of CD28 SEQ ID NO 4 |
| SEQ ID NO: 5 | AA 52-164 of the CD3ζ protein SEQ ID NO 5 |
| SEQ ID NO: 6 | PD1-CD28-CD3z SEQ ID NO 6 |
| SEQ ID NO: 7 | AA 239-304 of the NKp46 protein SEQ ID NO 7 |
| SEQ ID NO: 8 | PD1-NKp46 SEQ ID NO 8 |
| SEQ ID NO: 9 | full length DAP10 (AA 19-93) SEQ ID NO 9 |
| SEQ ID NO: 10 | DAP12 (AA 22-113) SEQ ID No: 10 |
| SEQ ID NO: 11 | PD1 AA sequence 1 to 212 SEQ ID NO 11 |
| SEQ ID NO: 12 | AA 77-93 of DAP10 SEQ ID NO 12 |
| SEQ ID NO: 13 | AA 73-113 of DAP12 SEQ ID NO 13 |
| SEQ ID NO: 14 | PD1EcDAP10TM-IC SEQ ID NO 14 |
| SEQ ID NO: 15 | PD1EcDAP12TM-IC SEQ ID NO 15 |
| SEQ ID NO: 16 | PD1EcTMDAP10IC SEQ ID NO: 16 |
| SEQ ID NO: 17 | PD1EcTMDAP12IC SEQ ID NO 17 |
| SEQ ID NO: 18 | LeGO-iG2 (Addgene plasmid 27341) SEQ ID NO 18 |
| SEQ ID NO: 19 | LeGO-MCS-GSG-T2A-eGFP SEQ ID NO 19 |
| SEQ ID NO: 20: | KO1 (guide RNA sequence TACCGCTGCATGATCAGCTATGG) |
| | (SEQ ID NO: 20) |
| SEQ ID NO: 21 | KO2 (guide RNA sequence: TACCATACTCTACCACATATAGG) |
| | (SEQ ID NO: 21) |
| SEQ ID NO: 22 | Sequence inserted into Vector MAP for vector LeGO_MCS-GSG-T2A- |
| | eGFP |

PD1_{EcTM} PD1_{EcTM}
SEQ ID NO 1
ggatccgccaccatgcagatcccacaggcgccctggccagtcgtctgggcggtgctacaactgggctggcggccaggatggttcttag
actccccagacaggccctggaacccccccaccttctccccagccctgctcgtggtgaccgaaggggacaacgccaccttcacctgca
gcttctccaacacatcggagagcttcgtgctaaactggtaccgcatgagccccagcaaccagacggacaagctggccgccttccccg
aggaccgcagccagcccggccaggactgccgcttccgtgtcacacaactgcccaacgggcgtgacttccacatgagcgtggtcaggg
cccggcgcaatgacagcggcacctacctctgtggggccatctccctggcccccaaggcgcagatcaaagagagcctgcgggcagag
ctcagggtgacagagagaagggcagaagtgcccacagcccaccccagcccctcacccaggccagccggccagttccaaaccctggt
ggttggtgtcgtgggcggcctgctgggcagcctggtgctgctagtctgggtcctggccgtcatctgctcccgggccgcacgagggaca
ataggagccaggcgcaccggccagcccctgaaggagtctagataagaattc
SEQ ID No 2
ATGCAGATCCCACAGGCGCCCTGGCCAGTCGTCTGGGCGGTGCTACAACTGGGCTGGCGGCCAGGAT
GGTTCTTAGACTCCCCAGACAGGCCCTGGAACCCCCCCACCTTCTCCCCAGCCCTGCTCGTGGTGACC
GAAGGGGACAACGCCACCTTCACCTGCAGCTTCTCCAACACATCGGAGAGCTTCGTGCTAAACTGGTA
CCGCATGAGCCCCAGCAACCAGACGGACAAGCTGGCCGCCTTCCCCGAGGACCGCAGCCAGCCCGGC
CAGGACTGCCGCTTCCGTGTCACACAACTGCCCAACGGGCGTGACTTCCACATGAGCGTGGTCAGGG
CCCGGCGCAATGACAGCGGCACCTACCTCTGTGGGGCCATCTCCCTGGCCCCCAAGGCGCAGATCAA
AGAGAGCCTGCGGGCAGAGCTCAGGGTGACAGAGAGAAGGGCAGAAGTGCCCACAGCCCACCCCA
GCCCCTCACCCAGGCCAGCCGGCCAGTTCCAAACCCTGGTG
SEQ ID No 3
CCACCACGACGCCAGCGCCGCGACCACCAACACCGGCGCCCACCATCGCGTCGCAGCCCCTGTCCCT
GCGCCCAGAGGCGTGCCGGCCAGCGGCGGGGGGCGCAGTGCACACGAGGGGGCTGGACTTCGCCC
CTAGGAAAATTGAAGTTATGTATCCTCCTCCTTACCTAGACAATGAGAAGAGCAATGGAACCATTATCC
ATGTGAAAGGGAAACACCTTTGTCCAAGTCCCCTATTTCCCGGACCTTCTAAGCCC
SEQ ID No 4
TTTTGGGTGCTGGTGGTGGTTGGTGGAGTCCTGGCTTGCTATAGCTTGCTAGTAACAGTGGCCTTTAT
TATTTTCTGGGTG
SEQ ID No 5
AGGAGTAAGAGGAGCAGGCTCCTGCACAGTGACTACATGAACATGACTCCCCGCCGCCCCGGGCCCA
CCCGCAAGCATTACCAGCCCTATGCCCCACCACGCGACTTCGCAGCCTATCGCTCCAGAGTGAAGTTC
AGCAGGAGCGCAGACGCCCCCGCGTACCAGCAGGGCCAGAACCAGCTCTATAACGAGCTCAATCTAG
GACGAAGAGAGGAGTACGATGTTTTGGACAAGAGACGTGGCCGGGACCCTGAGATGGGGGGAAAG
CCGAGAAGGAAGAACCCTCAGGAAGGCCTGTACAATGAACTGCAGAAAGATAAGATGGCGGAGGCC
TACAGTGAGATTGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCCCTCGCTAA

| | |
|---|---|
| SEQ ID NO 6 | |
| ggatccgcca ccatgcagat cccacaggcg ccctggccag tcgtctgggc ggtgctacaa | 60 |
| ctgggctggc ggccaggatg gttcttagac tccccagaca ggccctggaa cccccccacc | 120 |
| ttctccccag ccctgctcgt ggtgaccgaa ggggacaacg ccaccttcac ctgcagcttc | 180 |
| tccaacacat cggagagctt cgtgctaaac tggtaccgca tgagccccag caaccagacg | 240 |
| gacaagctgg ccgccttccc cgaggaccgc agccagcccg gccaggactg ccgcttccgt | 300 |
| gtcacacaac tgcccaacgg gcgtgacttc cacatgagcg tggtcagggc ccggcgcaat | 360 |
| gacagcggca cctacctctg tggggccatc tccctggccc ccaaggcgca gatcaaagag | 420 |
| agcctgcggg cagagctcag ggtgacagag agaagggcag aagtgcccac agcccacccc | 480 |
| agcccctcac ccaggccagc cggccagttc caaaccctgg tgctcgaggc taagcccacc | 540 |
| acgacgccag cgccgcgacc accaacaccg gcgcccacca tcgcgtcgca gcccctgtcc | 600 |
| ctgcgcccag aggcgtgccg gccagcggcg gggggcgcag tgcacacgag ggggctggac | 660 |
| ttcgccccta ggaaaattga agttatgtat cctcctcctt acctagacaa tgagaagagc | 720 |
| aatggaacca ttatccatgt gaaagggaaa cacctttgtc caagtcccct atttcccgga | 780 |
| ccttctaagc ccttttgggt gctggtggtg gttggtggag tcctggcttg ctatagcttg | 840 |
| ctagtaacag tggcctttat tattttctgg gtgaggagta agaggagcag gctcctgcac | 900 |
| agtgactaca tgaacatgac tccccgccgc cccgggccca cccgcaagca ttaccagccc | 960 |
| tatgccccac cacgcgactt cgcagcctat cgctccagag tgaagttcag caggagcgca | 1020 |
| gacgcccccg cgtaccagca gggccagaac cagctctata acgagctcaa tctaggacga | 1080 |
| agagaggagt acgatgtttt ggacaagaga cgtggccggg accctgagat ggggggaaag | 1140 |
| ccgagaagga agaaccctca ggaaggcctg tacaatgaac tgcagaaaga taagatggcg | 1200 |
| gaggcctaca gtgagattgg gatgaaaggc gagcgccgga ggggcaaggg gcacgatggc | 1260 |
| ctttaccagg gtctcagtac agccaccaag gacacctacg acgcccttca catgcaggcc | 1320 |
| ctgccccctc gctaagaatt c | 1341 |

Sequence ID No 7
Gagacgggactccagaaagaccatgccctctgggatcacactgcccagaatctccttcggatgggcctggcctttctagtcctggtgg
ctctagtgtggttcctggttgaagactggctcagcaggaagaggactagagagcgagccagcagagcttccacttgggaaggcagga
gaaggctgaacacacagactctttga
SEQ ID NO 8 PD1-NKp46 PD1-NKp46
GGATCCGCCACCATGCAGATCCCACAGGCGCCCTGGCCAGTCGTCTGGGCGGTGCTACAACTGGGCT
GGCGGCCAGGATGGTTCTTAGACTCCCCAGACAGGCCCTGGAACCCCCCCACCTTCTCCCCAGCCCTG
CTCGTGGTGACCGAAGGGGACAACGCCACCTTCACCTGCAGCTTCTCCAACACATCGGAGAGCTTCGT
GCTAAACTGGTACCGCATGAGCCCCAGCAACCAGACGGACAAGCTGGCCGCCTTCCCCGAGGACCGC
AGCCAGCCCGGCCAGGACTGCCGCTTCCGTGTCACACAACTGCCCAACGGGCGTGACTTCCACATGA
GCGTGGTCAGGGCCCGGCGCAATGACAGCGGCACCTACCTCTGTGGGGCCATCTCCCTGGCCCCCAA
GGCGCAGATCAAAGAGAGCCTGCGGGCAGAGCTCAGGGTGACAGAGAGAAGGGCAGAAGTGCCCA
CAGCCCACCCCAGCCCCTCACCCAGGCCAGCCGGCCAGTTCCAAACCCTGGTGctcgaggagacgggactcc
agaaagaccatgccctctgggatcacactgcccagaatctccttoggatgggcctggcctttctagtcctggtggctctagtgtggttcc
tggttgaagactggctcagcaggaagaggactagagagcgagccagcagagcttccacttgggaaggcaggagaaggctgaacac
acagactctttgatctagagaattc
Sequence ID No 9
cagacgactccaggagagagatcatcactccctgccttttaccctggcacttcaggctcttgttccggatgtgggtccctctctctgccg
ctcctggcaggcctcgtggctgctgatgcggtggcatcgctgctcatcgtgggggcggtgttcctgtgcgcacgcccacgccgcagccc
cgcccaagaagatggcaaagtctacatcaacatgccaggcaggggctga
Sequence ID No 10
Ctccgtcctgtccaggcccaggcccagagcgattgcagttgctctacggtgagcccgggcgtgctggcagggatcgtgatgggagac
ctggtgctgacagtgctcattgccctggccgtgtacttcctgggccggctggtccctcgggggcgaggggctgcggaggcagcgaccc
ggaaacagcgtatcactgagaccgagtcgccttatcaggagctccagggtcagaggtcggatgtctacagcgacctcaacacacaga
ggccgtattacaaatga
Sequence ID No 11
atgcagatcccacaggcgccctggccagtcgtctgggcggtgctacaactgggctggcggccaggatggttcttagactccccagaca
ggccctggaacccccccaccttctccccagccctgctcgtggtgaccgaaggggacaacgccaccttcacctgcagcttctccaacac
atcggagagcttcgtgctaaactggtaccgcatgagccccagcaaccagacggacaagctggccgccttccccgaggaccgcagcc
agcccggccaggactgccgcttccgtgtcacacaactgcccaacgggcgtgacttccacatgagcgtggtcagggcccggcgcaatg
acagcggcacctacctctgtggggccatctccctggcccccaaggcgcagatcaaagagagcctgcgggcagagctcagggtgaca
gagagaagggcagaagtgcccacagcccaccccagcccctcacccaggccagccggccagttccaaaccctggtggttggtgtcgt
gggcggcctgctgggcagcctggtgctgctagtctgggtcctggccgtcatctgctcccgggccgcacgagggacaataggagccag
gcgcaccggccagcccctgaaggag
Sequence ID No 12
agccccgcccaagaagatggcaaagtctacatcaacatgccaggcaggggctga
Sequence ID No 13
Ggggctgcggaggcagcgacccggaaacagcgtatcactgagaccgagtcgccttatcaggagctccagggtcagaggtcggatgt
ctacagcgacctcaacacacagaggccgtattacaaatga
SEQ ID NO: 14 PD1_{Ec}DAP10_{TM-IC}
GGATCCGCCACCATGCAGATCCCACAGGCGCCCTGGCCAGTCGTCTGGGCGGTGCTACAACTGGGCT
GGCGGCCAGGATGGTTCTTAGACTCCCCAGACAGGCCCTGGAACCCCCCCACCTTCTCCCCAGCCCTG
CTCGTGGTGACCGAAGGGGACAACGCCACCTTCACCTGCAGCTTCTCCAACACATCGGAGAGCTTCGT
GCTAAACTGGTACCGCATGAGCCCCAGCAACCAGACGGACAAGCTGGCCGCCTTCCCCGAGGACCGC
AGCCAGCCCGGCCAGGACTGCCGCTTCCGTGTCACACAACTGCCCAACGGGCGTGACTTCCACATGA
GCGTGGTCAGGGCCCGGCGCAATGACAGCGGCACCTACCTCTGTGGGGCCATCTCCCTGGCCCCCAA
GGCGCAGATCAAAGAGAGCCTGCGGGCAGAGCTCAGGGTGACAGAGAGAAGGGCAGAAGTGCCCA
CAGCCCACCCCAGCCCCTCACCCAGGCCAGCCGGCCAGTTCCAAACCCTGGTGctcgagcagacgactccag
gagagagatcatcactccctgccttttaccctggcacttcaggctcttgttccggatgtgggtccctctctctgccgctcctggcaggcct
cgtggctgctgatgcggtggcatcgctgctcatcgtgggggcggtgttcctgtgcgcacgcccacgccgcagccccgcccaagaagat
ggcaaagtctacatcaacatgccaggcaggggctgatctagagaattc
SEQ ID NO: 15
PD1_{Ec}DAP12_{TM-IC}
GGATCCGCCACCATGCAGATCCCACAGGCGCCCTGGCCAGTCGTCTGGGCGGTGCTACAACTGGGCT
GGCGGCCAGGATGGTTCTTAGACTCCCCAGACAGGCCCTGGAACCCCCCCACCTTCTCCCCAGCCCTG
CTCGTGGTGACCGAAGGGGACAACGCCACCTTCACCTGCAGCTTCTCCAACACATCGGAGAGCTTCGT
GCTAAACTGGTACCGCATGAGCCCCAGCAACCAGACGGACAAGCTGGCCGCCTTCCCCGAGGACCGC
AGCCAGCCCGGCCAGGACTGCCGCTTCCGTGTCACACAACTGCCCAACGGGCGTGACTTCCACATGA
GCGTGGTCAGGGCCCGGCGCAATGACAGCGGCACCTACCTCTGTGGGGCCATCTCCCTGGCCCCCAA
GGCGCAGATCAAAGAGAGCCTGCGGGCAGAGCTCAGGGTGACAGAGAGAAGGGCAGAAGTGCCCA
CAGCCCACCCCAGCCCCTCACCCAGGCCAGCCGGCCAGTTCCAAACCCTGGTGctcgagctccgtcctgtcca
ggcccaggcccagagcgattgcagttgctctacggtgagcccgggcgtgctggcagggatcgtgatgggagacctggtgctgacagt
gctcattgccctggccgtgtacttcctgggccggctggtccctcgggggcgaggggctgcggaggcagcgacccggaaacagcgtat
cactgagaccgagtcgccttatcaggagctccagggtcagaggtcggatgtctacagcgacctcaacacacagaggccgtattacaa
atgatctagagaattc
SEQ ID NO: 16
PD1_{EcTM}DAP10_{IC}
Ggatccgccaccatgcagatcccacaggcgccctggccagtcgtctgggcggtgctacaactgggctggcggccaggatggttctta
gactccccagacaggccctggaacccccccaccttctccccagccctgctcgtggtgaccgaaggggacaacgccaccttcacctgc
agcttctccaacacatcggagagcttcgtgctaaactggtaccgcatgagccccagcaaccagacggacaagctggccgccttcccc
gaggaccgcagccagcccggccaggactgccgcttccgtgtcacacaactgcccaacgggcgtgacttccacatgagcgtggtcagg
gcccggcgcaatgacagcggcacctacctctgtggggccatctccctggcccccaaggcgcagatcaaagagagcctgcgggcaga
gctcagggtgacagagagaagggcagaagtgcccacagcccaccccagcccctcacccaggccagccggccagttccaaaccctg
gtggttggtgtcgtgggcggcctgctgggcagcctggtgctgctagtctgggtcctggccgtcatctgctcccgggccgcacgagggac
aataggagccaggcgcaccggccagcccctgaaggagtctagaagccccgcccaagaagatggcaaagtctacatcaacatgcca
ggcaggggctgagaattc
SEQ ID NO: 17
PD1_{EcTM}DAP12_{IC}
ggatccgccaccatgcagatcccacaggcgccctggccagtcgtctgggcggtgctacaactgggctggcggccaggatggttcttag
actccccagacaggccctggaacccccccaccttctccccagccctgctcgtggtgaccgaaggggacaacgccaccttcacctgca
gcttctccaacacatcggagagcttcgtgctaaactggtaccgcatgagccccagcaaccagacggacaagctggccgccttccccg
aggaccgcagccagcccggccaggactgccgcttccgtgtcacacaactgcccaacgggcgtgacttccacatgagcgtggtcaggg
cccggcgcaatgacagcggcacctacctctgtggggccatctccctggcccccaaggcgcagatcaaagagagcctgcgggcagag
ctcagggtgacagagagaagggcagaagtgcccacagcccaccccagcccctcacccaggccagccggccagttccaaaccctggt
ggttggtgtcgtgggcggcctgctgggcagcctggtgctgctagtctgggtcctggccgtcatctgctcccgggccgcacgagggaca
ataggagccaggcgcaccggccagcccctgaaggagtctagaggggctgcggaggcagcgacccggaaacagcgtatcactgaga
ccgagtcgccttatcaggagctccagggtcagaggtcggatgtctacagcgacctcaacacacagaggccgtattacaaatgagaat
tc
SEQ ID NO: 18
LeGoiG2 (Addgene plasmid 27341)
gtcgacggatcgggagatctcccgatcccctatggtgcactctcagtacaatctgctctgatgccgcatagttaagccagtatctgctcc
ctgcttgtgtgttggaggtcgctgagtagtgcgcgagcaaaatttaagctacaacaaggcaaggcttgaccgacaattgcatgaagaa
tctgcttagggttaggcgttttgcgctgcttcgcgatgtacgggccagatatacgcgttgacattgattattgactagttattaatagtaa
tcaattacggggtcattagttcatagcccatatatggagttccgcgttacataacttacggtaaatggcccgcctggctgaccgcccaa
cgacccccgcccattgacgtcaataatgacgtatgttcccatagtaacgccaatagggactttccattgacgtcaatgggtggagtatt
tacggtaaactgcccacttggcagtacatcaagtgtatcatatgccaagtacgccccctattgacgtcaatgacggtaaatggcccgcc
tggcattatgcccagtacatgaccttatgggactttcctacttggcagtacatctacgtattagtcatcgctattaccatggtgatgcggt
tttggcagtacatcaatgggcgtggatagcggtttgactcacggggatttccaagtctccaccccattgacgtcaatgggagtttgtttt
ggcaccaaaatcaacgggactttccaaaatgtcgtaacaactccgccccattgacgcaaatgggcggtaggcgtgtacggtgggagg
tctatataagcagcgcgttttgcctgtactgggtctctctggttagaccagatctgagcctgggagctctctggctaactagggaaccca
ctgcttaagcctcaataaagcttgccttgagtgcttcaagtagtgtgtgcccgtctgttgtgtgactctggtaactagagatccctcagac
ccttttagtcagtgtggaaaatctctagcagtggcgcccgaacagggacttgaaagcgaaagggaaaccagaggagctctctcgacg
caggactcggcttgctgaagcgcgcacggcaagaggcgaggggcggcgactggtgagtacgccaaaaattttgactagcggaggct
agaaggagagagatgggtgcgagagcgtcagtattaagcgggggagaattagatcgcgatgggaaaaaattcggttaaggccagg
gggaaagaaaaaatataaattaaaacatatagtatgggcaagcagggagctagaacgattcgcagttaatcctggcctgttagaaac
atcagaaggctgtagacaaatactgggacagctacaaccatcccttcagacaggatcagaagaacttagatcattatataatacagt
agcaaccctctattgtgtgcatcaaaggatagagataaaagacaccaaggaagctttagacaagatagaggaagagcaaaacaaa
agtaagaccaccgcacagcaagcggccggccgcgctgatcttcagacctggaggaggagatatgagggacaattggagaagtgaa
ttatataaatataaagtagtaaaaattgaaccattaggagtagcacccaccaaggcaaagagaagagtggtgcagagagaaaaaa
gagcagtgggaataggagctttgttccttgggttcttgggagcagcaggaagcactatgggcgcagcgtcaatgacgctgacggtac
aggccagacaattattgtctggtatagtgcagcagcagaacaatttgctgagggctattgaggcgcaacagcatctgttgcaactcac
agtctggggcatcaagcagctccaggcaagaatcctggctgtggaaagatacctaaaggatcaacagctcctggggatttggggttg
ctctggaaaactcatttgcaccactgctgtgccttggaatgctagttggagtaataaatctctggaacagatttggaatcacacgacct
ggatggagtgggacagagaaattaacaattacacaagcttaatacactccttaattgaagaatcgcaaaaccagcaagaaaagaat
gaacaagaattattggaattagataaatgggcaagtttgtggaattggtttaacataacaaattggctgtggtatataaaattattcata
atgatagtaggaggcttggtaggtttaagaatagtttttgctgtactttctatagtgaatagagttaggcagggatattcaccattatcgt
ttcagacccacctcccaaccccgaggggacccgacaggcccgaaggaatagaagaagaaggtggagagagagacagagacagat
ccattcgattagtgaacggatcggcactgcgtgcgccaattctgcagacaaatggcagtattcatccacaattttaaaagaaaagggg
ggattggggggtacagtgcaggggaaagaatagtagacataatagcaacagacatacaaactaaagaattacaaaaacaaattac
aaaaattcaaaattttcgggtttattacagggacagcagagatccagtttggttagtaccgggcccgctctagtcgaggtcgacggtat
cgataagctcgcttcacgagattccagcaggtcgagggacctaataacttcgtatagcatacattatacgaagttatattaagggttcc
aagcttaagcggccggccgctgaaagaccccacctgtaggtttggcaagctagctgcagtaacgccattttgcaaggcatggaaaaa
taccaaaccaagaatagagaagttcagatcaagggcgggtacatgaaaatagctaacgttgggccaaacaggatatctgcggtgag
cagtttcggccccggcccggggccaagaacagatggtcaccgcagtttcggccccggcccgaggccaagaacagatggtccccaga
tatggcccaaccctcagcagtttcttaagacccatcagatgtttccaggctcccccaaggacctgaaatgaccctgcgccttatttgaat
taaccaatcagcctgcttctcgcttctgttcgcgcgcttctgcttcccgagctctataaaagagctcacaacccctcactcggcgcgcca
gtcctccgattgactgagtcgcccggatcccagtgtggtggtacgggaattcctgcaggcctcgacgagggccggcgcgccgcggcc
gctacgtaaattccgccccccccccccctctccctcccccccccctaacgttactggccgaagccgcttggaataaggccggtgtgcgt
ttgtctatatgttattttccaccatattgccgtcttttggcaatgtgagggcccggaaacctggccctgtcttcttgacgagcattcctagg
ggtctttcccctctcgccaaaggaatgcaaggtctgttgaatgtcgtgaaggaagcagttcctctggaagcttcttgaagacaaacaac
gtctgtagcgaccctttgcaggcagcggaaccccccacctggcgacaggtgcctctgcggccaaaagccacgtgtataagatacacc
tgcaaaggcggcacaaccccagtgccacgttgtgagttggatagttgtggaaagagtcaaatggctctcctcaagcgtattcaacaag
gggctgaaggatgcccagaaggtaccccattgtatgggatctgatctggggcctcggtgcacatgctttacatgtgtttagtcgaggtt
aaaaaaacgtctaggccccccgaaccacggggacgtggttttcctttgaaaaacacgatgataatatggccacaaccatggtgagca
agggcgaggagctgttcaccggggtggtgcccatcctggtcgagctggacggcgacgtaaacggccacaagttcagcgtgtccggcg
agggcgagggcgatgccacctacggcaagctgaccctgaagttcatctgcaccaccggcaagctgcccgtgccctggcccaccctcg
tgaccaccctgacctacggcgtgcagtgcttcagccgctaccccgaccacatgaagcagcacgacttcttcaagtccgccatgcccga
aggctacgtccaggagcgcaccatcttcttcaaggacgacggcaactacaagacccgcgccgaggtgaagttcgagggcgacaccc
tggtgaaccgcatcgagctgaagggcatcgacttcaaggaggacggcaacatcctggggcacaagctggagtacaactacaacagc
cacaacgtctatatcatggccgacaagcagaagaacggcatcaaggtgaacttcaagatccgccacaacatcgaggacggcagcgt
gcagctcgccgaccactaccagcagaacacccccatcggcgacggccccgtgctgctgcccgacaaccactacctgagcacccagtc
cgccctgagcaaagaccccaacgagaagcgcgatcacatggtcctgctggagttcgtgaccgccgccgggatcactctcggcatgga
cgagctgtacaagtaaagcggccggccgccagcacagtggtcgaaattcgtcgagggacctaataacttcgtatagcatacattatac
gaagttatacatgtttaagggttccggttccactaggtacaattcgatatcaagcttatcgataatcaacctctggattacaaaatttgtg
aaagattgactggtattcttaactatgttgctccttttacgctatgtggatacgctgctttaatgcctttgtatcatgctattgcttcccgtat
ggctttcattttctcctccttgtataaatcctggttgctgtctctttatgaggagttgtggcccgttgtcaggcaacgtggcgtggtgtgca
ctgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcgggga
aatcatcgtcctttccttggctgctcgcctgtgttgccacctggattctgcgcgggacgtccttctgctacgtcccttcggccctcaatcca
gcggaccttccttcccgcggcctgctgccggctctgcggcctcttccgcgtcttcgccttcgccctcagacgagtcggatctccctttggg
ccgcctccccgcatcgataccgtcgacctcgatcgagacctagaaaaacatggagcaatcacaagtagcaatacagcagctaccaat
gctgattgtgcctggctagaagcacaagaggaggaggaggtgggttttccagtcacacctcaggtacctttaagaccaatgacttaca
aggcagctgtagatcttagccactttttaaaagaaaaggggggactggaagggctaattcactcccaacgaagacaagatatccttg
atctgtggatctaccacacacaaggctacttccctgattggcagaactacacaccagggccagggatcagatatccactgacctttgg
atggtgctacaagctagtaccagttgagcaagagaaggtagaagaagccaatgaaggagagaacacccgcttgttacaccctgtga
gcctgcatgggatggatgacccggagagagaagtattagagtggaggtttgacagccgcctagcatttcatcacatggcccgagagc
tgcatccggactgtactgggtctctctggttagaccagatctgagcctgggagctctctggctaactagggaacccactgcttaagcct
caataaagcttgccttgagtgcttcaagtagtgtgtgcccgtctgttgtgtgactctggtaactagagatccctcagacccttttagtcag
tgtggaaaatctctagcagcatgtgagcaaaaggccagcaaaaggccaggaaccgtaaaaaggccgcgttgctggcgtttttccata
ggctccgcccccctgacgagcatcacaaaaatcgacgctcaagtcagaggtggcgaaacccgacaggactataaagataccaggcg
tttccccctggaagctccctcgtgcgctctcctgttccgaccctgccgcttaccggatacctgtccgcctttctcccttcgggaagcgtgg
cgctttctcatagctcacgctgtaggtatctcagttcggtgtaggtcgttcgctccaagctgggctgtgtgcacgaaccccccgttcagc
ccgaccgctgcgccttatccggtaactatcgtcttgagtccaacccggtaagacacgacttatcgccactggcagcagccactggtaa
caggattagcagagcgaggtatgtaggcggtgctacagagttcttgaagtggtggcctaactacggctacactagaagaacagtattt
ggtatctgcgctctgctgaagccagttaccttcggaaaaagagttggtagctcttgatccggcaaacaaaccaccgctggtagcggtg
gtttttttgtttgcaagcagcagattacgcgcagaaaaaaaggatctcaagaagatcctttgatcttttctacggggtctgacgctcagt
ggaacgaaaactcacgttaagggattttggtcatgagattatcaaaaaggatcttcacctagatccttttaaattaaaaatgaagtttta
aatcaatctaaagtatatatgagtaaacttggtctgacagttaccaatgcttaatcagtgaggcacctatctcagcgatctgtctatttc
gttcatccatagttgcctgactccccgtcgtgtagataactacgatacgggagggcttaccatctggccccagtgctgcaatgataccg
cgagacccacgctcaccggctccagatttatcagcaataaaccagccagccggaagggccgagcgcagaagtggtcctgcaacttta
tccgcctccatccagtctattaattgttgccgggaagctagagtaagtagttcgccagttaatagtttgcgcaacgttgttgccattgcta
caggcatcgtggtgtcacgctcgtcgtttggtatggcttcattcagctccggttcccaacgatcaaggcgagttacatgatcccccatgt
tgtgcaaaaaagcggttagctccttcggtcctccgatcgttgtcagaagtaagttggccgcagtgttatcactcatggttatggcagcac
tgcataattctcttactgtcatgccatccgtaagatgcttttctgtgactggtgagtactcaaccaagtcattctgagaatagtgtatgcg
gcgaccgagttgctcttgcccggcgtcaatacgggataataccgcgccacatagcagaactttaaaagtgctcatcattggaaaacgt
tcttcggggcgaaaactctcaaggatcttaccgctgttgagatccagttcgatgtaacccactcgtgcacccaactgatcttcagcatct
tttactttcaccagcgtttctgggtgagcaaaaacaggaaggcaaaatgccgcaaaaaagggaataagggcgacacggaaatgttg
aatactcatactcttcctttttcaatattattgaagcatttatcagggttattgtctcatgagcggatacatatttgaatgtatttagaaaa
ataaacaaataggggttccgcgcacatttccccgaaaagtgccacctgac (SEQ ID NO: 18)
SEQ ID NO: 19:
LeGO-MCS-GSG-T2A-eGFP
gtcgacggatcgggagatctcccgatcccctatggtgcactctcagtacaatctgctctgatgccgcatagttaagccagtatctgctccc
tgcttgtgtgttggaggtcgctgagtagtgcgcgagcaaaatttaagctacaacaaggcaaggcttgaccgacaattgcatgaagaatct
gcttagggttaggcgttttgcgctgcttcgcgatgtacgggccagatatacgcgttgacattgattattgactagttattaatagtaatcaatt
acggggtcattagttcatagcccatatatggagttccgcgttacataacttacggtaaatggcccgcctggctgaccgcccaacgacccc
cgcccattgacgtcaataatgacgtatgttcccatagtaacgccaatagggactttccattgacgtcaatgggtggagtatttacggtaaa
ctgcccacttggcagtacatcaagtgtatcatatgccaagtacgccccctattgacgtcaatgacggtaaatggcccgcctggcattatg
cccagtacatgaccttatgggactttcctacttggcagtacatctacgtattagtcatcgctattaccatggtgatgcggttttggcagtacat
caatgggcgtggatagcggtttgactcacggggatttccaagtctccaccccattgacgtcaatgggagtttgttttggcaccaaaatcaa
cgggactttccaaaatgtcgtaacaactccgccccattgacgcaaatgggcggtaggcgtgtacggtgggaggtctatataagcagcg
cgttttgcctgtactgggtctctctggttagaccagatctgagcctgggagctctctggctaactagggaacccactgcttaagcctcaata
aagcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatataa
attaaaacatatagtatgggcaagcagggagctagaacgattcgcagttaatcctggcctgttagaaacatcagaaggctgtagacaaa
tactgggacagctacaaccatcccttcagacaggatcagaagaacttagatcattatataatacagtagcaaccctctattgtgtgcatca
aaggatagagataaaagacaccaaggaagctttagacaagatagaggaagagcaaaacaaaagtaagaccaccgcacagcaagcg
gccggccgcgctgatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagctttgttccttg
ggttcttgggagcagcaggaagcactatgggcgcagcgtcaatgacgctgacggtacaggccagacaattattgtctggtatagtgca
gcagcagaacaatttgctgagggctattgaggcgcaacagcatctgttgcaactcacagtctggggcatcaagcagctccaggcaaga
atcctggctgtggaaagatacctaaaggatcaacagctcctggggatttggggttgctctggaaaactcatttgcaccactgctgtgcctt
ggaatgctagttggagtaataaatctctggaacagatttggaatcacacgacctggatggagtgggacagagaaattaacaattacaca
agcttaatacactccttaattgaagaatcgcaaaaccagcaagaaaagaatgaacaagaattattggaattagataaatgggcaagtttgt
ggaattggtttaacataacaaattggctgtggtatataaaattattcataatgatagtaggaggcttggtaggtttaagaatagtttttgctgta
ctttctatagtgaatagagttaggcagggatattcaccattatcgtttcagacccacctcccaaccccgaggggacccgacaggcccga
aggaatagaagaagaaggtggagagagagacagagacagatccattcgattagtgaacggatcggcactgcgtgcgccaattctgc
agacaaatggcagtattcatccacaattttaaaagaaaaggggggattggggggtacagtgcaggggaaagaatagtagacataatag
caacagacatacaaactaaagaattacaaaaacaaattacaaaaattcaaaattttcgggtttattacagggacagcagagatccagtttg
gttagtaccgggcccgctctagtcgaggtcgacggtatcgataagctcgcttcacgagattccagcaggtcgagggacctaataacttc
gtatagcatacattatacgaagttatattaagggttccaagcttaagcggccgctgaaagaccccacctgtaggtttggcaagctagctgc
agtaacgccattttgcaaggcatggaaaaataccaaaccaagaatagagaagttcagatcaagggcgggtacatgaaaatagctaacg
ttgggccaaacaggatatctgcggtgagcagtttcggccccggcccggggccaagaacagatggtcaccgcagtttcggccccggc
ccgaggccaagaacagatggtccccagatatggcccaaccctcagcagtttcttaagacccatcagatgtttccaggctcccccaagg
acctgaaatgaccctgcgccttatttgaattaaccaatcagcctgcttctcgcttctgttcgcgcgcttctgcttcccgagctctataaaaga
gctcacaacccctcactcggcgcgccagtcctccgattgactgagtcgcccGGATCCGCCACCATGGCTAAGCT
AGGAAGCGGAGAGGGCAGAGGCAGTCTGCTGACATGCGGTGACGTGGAAGAGA
ATCCCGGCCCTgtgagcaagggcgaggagctgttcaccggggtggtgcccatcctggtcgagctggacggcgacgtaaac
ggccacaagttcagcgtgtccggcgagggcgagggcgatgccacctacggcaagctgaccctgaagttcatctgcaccaccggcaa
gctgcccgtgccctggcccaccctcgtgaccaccctgacctacggcgtgcagtgcttcagccgctaccccgaccacatgaagcagca
cgacttcttcaagtccgccatgcccgaaggctacgtccaggagcgcaccatcttcttcaaggacgacggcaactacaagacccgcgc
cgaggtgaagttcgagggcgacaccctggtgaaccgcatcgagctgaagggcatcgacttcaaggaggacggcaacatcctgggg
cacaagctggagtacaactacaacagccacaacgtctatatcatggccgacaagcagaagaacggcatcaaggtgaacttcaagatc
cgccacaacatcgaggacggcagcgtgcagctcgccgaccactaccagcagaacacccccatcggcgacggccccgtgctgctgc
ccgacaaccactacctgagcacccagtccgccctgagcaaagaccccaacgagaagcgcgatcacatggtcctgctggagttcgtga
ccgccgccgggatcactctcggcatggacgagctgtacaagTAAGAATTCgtcgagggacctaataacttcgtatagcataca
ttatacgaagttatacatgtttaagggttccggttccactaggtacaattcgatatcaagcttatcgataatcaacctctggattacaaaatttg
tgaaagattgactggtattcttaactatgttgctccttttacgctatgtggatacgctgctttaatgcctttgtatcatgctattgcttcccgtatg
gctttcattttctcctccttgtataaatcctggttgctgtctctttatgaggagttgtggcccgttgtcaggcaacgtggcgtggtgtgcactgt
gtttgctgacgcaacccccactggttggggcattgccaccacctgtcagctcctttccgggactttcgctttccccctccctattgccacgg
cggaactcatcgccgcctgccttgcccgctgctggacaggggctcggctgttgggcactgacaattccgtggtgttgtcggggaaatc
atcgtcctttccttggctgctcgcctgtgttgccacctggattctgcgcgggacgtccttctgctacgtcccttcggccctcaatccagcgg
accttccttcccgcggcctgctgccggctctgcggcctcttccgcgtcttcgccttcgccctcagacgagtcggatctccctttgggccg
cctccccgcatcgataccgtcgacctcgatcgagacctagaaaaacatggagcaatcacaagtagcaatacagcagctaccaatgctg
attgtgcctggctagaagcacaagaggaggaggaggtgggttttccagtcacacctcaggtacctttaagaccaatgacttacaaggca
gctgtagatcttagccactttttaaaagaaaaggggggactggaagggctaattcactcccaacgaagacaagatatccttgatctgtgg
atctaccacacacaaggctacttccctgattggcagaactacacaccagggccagggatcagatatccactgacctttggatggtgcta
caagctagtaccagttgagcaagagaaggtagaagaagccaatgaaggagagaacacccgcttgttacaccctgtgagcctgcatgg
gatggatgacccggagagagaagtattagagtggaggtttgacagccgcctagcatttcatcacatggcccgagagctgcatccggac
tgtactgggtctctctggttagaccagatctgagcctgggagctctctggctaactagggaacccactgcttaagcctcaataaagcttgc
cttgagtgcttcaagtagtgtgtgcccgtctgttgtgtgactctggtaactagagatccctcagacccttttagtcagtgtggaaaatctcta
gcagcatgtgagcaaaaggccagcaaaaggccaggaaccgtaaaaaggccgcgttgctggcgtttttccataggctccgcccccctg
acgagcatcacaaaaatcgacgctcaagtcagaggtggcgaaacccgacaggactataaagataccaggcgtttccccctggaagct
ccctcgtgcgctctcctgttccgaccctgccgcttaccggatacctgtccgcctttctcccttcgggaagcgtggcgctttctcatagctca
cgctgtaggtatctcagttcggtgtaggtcgttcgctccaagctgggctgtgtgcacgaaccccccgttcagcccgaccgctgcgcctta
tccggtaactatcgtcttgagtccaacccggtaagacacgacttatcgccactggcagcagccactggtaacaggattagcagagcga
ggtatgtaggcggtgctacagagttcttgaagtggtggcctaactacggctacactagaagaacagtatttggtatctgcgctctgctgaa
gccagttaccttcggaaaaagagttggtagctcttgatccggcaaacaaaccaccgctggtagcggtggtttttttgtttgcaagcagcag
attacgcgcagaaaaaaaggatctcaagaagatcctttgatcttttctacggggtctgacgctcagtggaacgaaaactcacgttaaggg
attttggtcatgagattatcaaaaaggatcttcacctagatccttttaaattaaaaatgaagttttaaatcaatctaaagtatatatgagtaaact
tggtctgacagttaccaatgcttaatcagtgaggcacctatctcagcgatctgtctatttcgttcatccatagttgcctgactccccgtcgtgt
agataactacgatacgggagggcttaccatctggccccagtgctgcaatgataccgcgagacccacgctcaccggctccagatttatc
agcaataaaccagccagccggaagggccgagcgcagaagtggtcctgcaactttatccgcctccatccagtctattaattgttgccggg
aagctagagtaagtagttcgccagttaatagtttgcgcaacgttgttgccattgctacaggcatcgtggtgtcacgctcgtcgtttggtatg
gcttcattcagctccggttcccaacgatcaaggcgagttacatgatcccccatgttgtgcaaaaaagcggttagctccttcggtcctccga
tcgttgtcagaagtaagttggccgcagtgttatcactcatggttatggcagcactgcataattctcttactgtcatgccatccgtaagatgctt
ttctgtgactggtgagtactcaaccaagtcattctgagaatagtgtatgcggcgaccgagttgctcttgcccggcgtcaatacgggataat
accgcgccacatagcagaactttaaaagtgctcatcattggaaaacgttcttcggggcgaaaactctcaaggatcttaccgctgttgaga
tccagttcgatgtaacccactcgtgcacccaactgatcttcagcatcttttactttcaccagcgtttctgggtgagcaaaaacaggaaggc
aaaatgccgcaaaaaagggaataagggcgacacggaaatgttgaatactcatactcttcctttttcaatattattgaagcatttatcagggtt
attgtctcatgagcggatacatatttgaatgtatttagaaaaataaacaaataggggttccgcgcacatttccccgaaaagtgccacctga
c
SEQ ID NO: 20
KO1 guide RNA sequence
TACCGCTGCATGATCAGCTATGG
SEQ ID NO: 21
KO2 guide RNA sequence:
TACCATACTCTACCACATATAGG
SEQ ID NO: 22 Sequence inserted into Vector MAP for vector LeGO_MCS-GSG-T2A-eGFP
GGATCCGCCACCATGGCTAAGCTAGGAAGCGGAGAGGGCAGAGGCAGTCTGCTGACATGCGGTGAC
GTGGAAGAGAATCCCGGCCCTgtgagcaagggcgaggagctgttcaccggggtggtgcccatcctggtcgagctggacgg
cgacgtaaacggccacaagttcagcgtgtccggcgagggcgagggcgatgccacctacggcaagctgaccctgaagttcatctgcac
caccggcaagctgcccgtgccctggcccaccctcgtgaccaccctgacctacggcgtgcagtgcttcagccgctaccccgaccacatg
aagcagcacgacttcttcaagtccgccatgcccgaaggctacgtccaggagcgcaccatcttcttcaaggacgacggcaactacaag
acccgcgccgaggtgaagttcgagggcgacaccctggtgaaccgcatcgagctgaagggcatcgacttcaaggaggacggcaacat
cctggggcacaagctggagtacaactacaacagccacaacgtctatatcatggccgacaagcagaagaacggcatcaaggtgaact
tcaagatccgccacaacatcgaggacggcagcgtgcagctcgccgaccactaccagcagaacacccccatcggcgacggccccgtg
ctgctgcccgacaaccactacctgagcacccagtccgccctgagcaaagaccccaacgagaagcgcgatcacatggtcctgctggag
ttcgtgaccgccgccgggatcactctcggcatggacgagctgtacaagTAAGAATTC

### REFERENCES

1. Ren, D., et al., Predictive biomarkers and mechanisms underlying resistance to PD1 PD-L1 blockade cancer immunotherapy. Mol Cancer, 2020. 19 (1): p. 19.
2. Kumpers, C., et al., Immune Cell Infiltration of the Primary Tumor, Not PD-L1 Status, Is Associated With Improved Response to Checkpoint Inhibition in Metastatic Melanoma. Front Med (Lausanne), 2019. 6: p. 27.
3. Zhu, H., et al., EGFRvIII-CAR-T Cells with PD-1 Knockout Have Improved Anti-Glioma Activity. Pathol Oncol Res, 2020. 26 (4): p. 2135-2141.
4. Rupp, L. J., et al., CRISPR Cas9-mediated PD-1 disruption enhances anti-tumor efficacy of human chimeric antigen receptor T cells. Sci Rep, 2017. 7 (1): p. 737.
5. McGowan, E., et al., PD-1 disrupted CAR-T cells in the treatment of solid tumors: Promises and challenges. Biomed Pharmacother, 2020. 121: p. 109625.
6. Wei, J., et al., PD-1 silencing impairs the anti-tumor function of chimeric antigen receptor modified T cells by inhibiting proliferation activity. J Immunother Cancer, 2019. 7 (1): p. 209.
7. Malmberg, K. J., et al., Natural killer cell-mediated immunosurveillance of human cancer. Semin Immunol, 2017. 31: p. 20-29.
8. Fauriat, C., et al., Regulation of human NK-cell cytokine and chemokine production by target cell recognition. Blood, 2010. 115 (11): p. 2167-76.
9. Lanier, L. L., Up on the tightrope: natural killer cell activation and inhibition. Nat Immunol, 2008. 9 (5): p. 495-502.
10. Wu, J., et al., DAP 10 and DAP12 form distinct, but functionally cooperative, receptor complexes in natural killer cells. J Exp Med, 2000. 192 (7): p. 1059-68.
11. Lanier, L. L., DAP 10-and DAP12-associated receptors in innate immunity. Immunol Rev, 2009. 227 (1): p. 150-60.
12. Liu, E., et al., Use of CAR-Transduced Natural Killer Cells in CD19-Positive Lymphoid Tumors. N Engl J Med, 2020. 382 (6): p. 545-553.
13. Xie, G., et al., CAR-NK cells: A promising cellular immunotherapy for cancer. EBioMedicine, 2020. 59: p. 102975.
14. Liu, Y., et al., Increased expression of programmed cell death protein 1 on NK cells inhibits NK-cell-mediated anti-tumor function and indicates poor prognosis in digestive cancers. Oncogene, 2017. 36 (44): p. 6143-6153.
15. Benson, D. M., Jr., et al., The PD-1 PD-L1 axis modulates the natural killer cell versus multiple myeloma effect: a therapeutic target for CT-011, a novel monoclonal anti-PD-1 antibody. Blood, 2010. 116 (13): p. 2286-94.
16. Concha-Benavente, F., et al., PD-L1 Mediates Dysfunction in Activated PD-1(+) NK Cells in Head and Neck Cancer Patients. Cancer Immunol Res, 2018. 6 (12): p. 1548-1560.
17. Davis, Z., et al., Low-density PD-1 expression on resting human natural killer cells is functional and upregulated after transplantation. Blood Adv, 2021. 5 (4): p. 1069-1080.
18. Wagner, A. K., et al., PD-1 expression on mouse intratumoral NK cells and its effects on NK cell phenotype. iScience, 2022. 25 (10): p. 105137.
19. Pessino, A., et al., Molecular cloning of NKp46: a novel member of the immunoglobulin superfamily involved in triggering of natural cytotoxicity. J Exp Med, 1998. 188 (5): p. 953-60.
20. Weber, K., et al., A multicolor panel of novel lentiviral “gene ontology” (LeGO) vectors for functional gene analysis. Mol Ther, 2008. 16 (4): p. 698-706.
21. Ran, F. A., et al., Genome engineering using the CRISPR-Cas9 system. Nat Protoc, 2013. 8 (11): p. 2281-2308.
22. Stewart, S. A., et al., Lentivirus-delivered stable gene silencing by RNAi in primary cells. RNA, 2003. 9 (4): p. 493-501.
23. Dull, T., et al., A third-generation lentivirus vector with a conditional packaging system. J Virol, 1998. 72 (11): p. 8463-71.
24. Sutlu, T., et al., Inhibition of intracellular antiviral defense mechanisms augments lentiviral transduction of human natural killer cells: implications for gene therapy. Hum Gene Ther, 2012. 23 (10): p. 1090-100.
25. Schwietzer, Y. A., et al., A tractable microscopy-and flow cytometry-based method to measure natural killer cell-mediated killing and infiltration of tumor spheroids, in Methods in Cell Biology. 2022, Academic Press.
26. Martins, F., et al., Adverse effects of immune-checkpoint inhibitors: epidemiology, management and surveillance. Nat Rev Clin Oncol, 2019. 16 (9): p. 563-580.
27. Fabian, K. P., et al., PD-L1 targeting high-affinity NK (t-haNK) cells induce direct antitumor effects and target suppressive MDSC populations. J Immunother Cancer, 2020. 8 (1).
28. Yearley, J. H., et al., PD-L2 Expression in Human Tumors: Relevance to Anti-PD-1 Therapy in Cancer. Clin Cancer Res, 2017. 23 (12): p. 3158-3167.
29. Brito, A. B. C., M. P. G. Camandaroba, and V. C. C. de Lima, Anti-PD1 versus anti-PD-L1 immunotherapy in first-line therapy for advanced non-small cell lung cancer: A systematic review and meta-analysis. Thorac Cancer, 2021. 12 (7): p. 1058-1066.
30. Andre, P., et al., Anti-NKG2A mAb Is a Checkpoint Inhibitor that Promotes Anti-tumor Immunity by Unleashing Both T and NK Cells. Cell, 2018. 175 (7): p. 1731-1743 e13.
31. Cao, Y., et al., Immune checkpoint molecules in natural killer cells as potential targets for cancer immunotherapy. Signal Transduct Target Ther, 2020. 5 (1): p. 250.
32. Bexte, T., et al., CRISPR-Cas9 based gene editing of the immune checkpoint NKG2A enhances NK cell mediated cytotoxicity against multiple myeloma. Oncoimmunology, 2022. 11 (1): p. 2081415.
33. Zhang, Q., et al., Blockade of the checkpoint receptor TIGIT prevents NK cell exhaustion and elicits potent anti-tumor immunity. Nat Immunol, 2018. 19 (7): p. 723-732.
34. Julia, E. P., et al., Avelumab, an IgG1 anti-PD-L1 Immune Checkpoint Inhibitor, Triggers NK Cell-Mediated Cytotoxicity and Cytokine Production Against Triple Negative Breast Cancer Cells. Front Immunol, 2018. 9: p. 2140.
35. Guo, C., et al., Structure-based rational design of a novel chimeric PD1-NKG2D receptor for natural killer cells. Mol Immunol, 2019. 114: p. 108-113.
36. Garrity, D., et al., The activating NKG2D receptor assembles in the membrane with two signaling dimers into a hexameric structure. Proc Natl Acad Sci USA, 2005. 102 (21): p. 7641-6.
37. Topfer, K., et al., DAP 12-based activating chimeric antigen receptor for NK cell tumor immunotherapy. J Immunol, 2015. 194 (7): p. 3201-12.
38. Nijhof, I. S., et al., Preclinical Evidence for the Therapeutic Potential of CD38-Targeted Immuno-Chemotherapy in Multiple Myeloma Patients Refractory to Lenalidomide and Bortezomib. Clin Cancer Res, 2015. 21 (12): p. 2802-10.
39. Mateos, M. V., et al., Daratumumab plus Bortezomib, Melphalan, and Prednisone for Untreated Myeloma. N Engl J Med, 2018. 378 (6): p. 518-528.
40. Alici, E., et al., Autologous antitumor activity by NK cells expanded from myeloma patients using GMP-compliant components. Blood, 2008. 111 (6): p. 3155-62.
41. Alici, E., et al., Anti-myeloma activity of endogenous and adoptively transferred activated natural killer cells in experimental multiple myeloma model. Exp Hematol, 2007. 35 (12): p. 1839-46.
42. Lesokhin, A. M., et al., Nivolumab in Patients With Relapsed or Refractory Hematologic Malignancy: Preliminary Results of a Phase Ib Study. J Clin Oncol, 2016. 34 (23): p. 2698-704.
43. Mateos, M. V., et al., Pembrolizumab plus pomalidomide and dexamethasone for patients with relapsed or refractory multiple myeloma (KEYNOTE-183): a randomised, open-label, phase 3 trial. Lancet Haematol, 2019. 6 (9): p. e459-e469.
44. Usmani, S. Z., et al., Pembrolizumab plus lenalidomide and dexamethasone for patients with treatment-naive multiple myeloma (KEYNOTE-185): a randomised, open-label, phase 3 trial. Lancet Haematol, 2019. 6 (9): p. e448-e458.
45. Kazandjian, D., et al., Avelumab, a PD-L1 Inhibitor, in Combination with Hypofractionated Radiotherapy and the Abscopal Effect in Relapsed Refractory Multiple Myeloma. Oncologist, 2021. 26 (4): p. 288-e541.

We claim:

1. An NK cell specific PD1-based chimeric switch receptor.

2. An NK cell comprising the chimeric switch receptor of claim 1, which chimeric switch receptor optionally contains at least one signaling domain from DAP10, DAP12, NKp46 or CD3ζ.

3. The NK cell of claim 2 wherein the chimeric switch receptor comprises amino acids 1-170 of PD1 (SEQ ID NO: 2), an 85 amino acid long hinge region (SEQ ID NO 3), amino acids 153-220 of CD28 (SEQ ID NO: 4) and amino acids 52-164 of the CD3ζ protein (SEQ ID NO: 5).

4. The NK cell of claim 2 wherein the chimeric switch receptor comprises amino acids 1-170 of PD1 (SEQ ID NO 2) fused together with amino acids 239-304 of the NKp46 protein (SEQ ID NO 7).

5. The NK cell of claim 2 wherein the chimeric switch receptor comprises amino acids 1 to 170 of PD1 (SEQ ID NO: 2) fused to full length DAP10 (AA 19-93) (SEQ ID NO: 6).

6. The NK cell of claim 2 wherein the chimeric switch receptor comprises amino acids 1 to 170 of PD1 (SEQ ID NO: 2) fused to the full length DAP12 (AA 22-113) (SEQ ID NO: 7) protein.

7. The NK cell of claim 2 wherein the chimeric switch receptor comprises amino acids 1 to 212 of PD1 (SEQ ID NO: 11) fused together with AA 77-93 of DAP10 (SEQ ID NO: 12)

8. The NK cell of claim 2 wherein the chimeric switch receptor comprises amino acids 1 to 212 of PD1 (SEQ ID NO: 11) fused together with amino acids 73-113 of DAP12 (SEQ ID NO 13).

9. The NK cell of claim 2 wherein the chimeric switch receptor comprises a signaling domain and the signaling domain is truncated, preferably wherein the signaling domain is truncated to omit the transmembrane region.

10. The NK cell of claim 2 wherein the chimeric switch receptor comprises amino acids 52-164 of the CD3ζ protein (SEQ ID NO: 5).

11. The NK Cell of claim 2 wherein the chimeric switch receptor comprises amino acids 239-304 of the NKp46 protein (SEQ ID NO 7).

12. The NK cell of claim 2 wherein the NK cell comprises the DAP 10 construct of SEQ ID NO 14.

13. The NK cell of claim 2 wherein the NK cell comprises the DAP 12 construct of SEQ ID NO 15.

14. The NK cell of claim 2 wherein the NK cell comprises the DAP 10 construct of SEQ ID NO 16.

15. The NK cell of claim 2 wherein the NK cell comprises the DAP 12 construct of SEQ ID NO 17.

16. A method of treating multiple myeloma in a patient in need thereof comprising administering an NK cell specific PD1-based chimeric switch receptor.

17. The method of claim 16 wherein the chimeric switch receptor comprises at least one signaling domain from DAP10, DAP12, NKp46 or CD3ζ.

18. The method of claim 17 wherein the chimeric switch receptor comprises amino acids 1-170 of PD1 (SEQ ID NO: 2), an 85 amino acid long hinge region (SEQ ID NO 3), amino acids 153-220 of CD28 (SEQ ID NO: 4) and amino acids 52-164 of the CD3ζ protein (SEQ ID NO: 5).

19. The method of claim 17 wherein the chimeric switch receptor comprises amino acids 1-170 of PD1 (SEQ ID NO 2) fused together with amino acids 239-304 of the NKp46 protein (SEQ ID NO 7).

20. The method of claim 17 wherein the chimeric switch receptor comprises amino acids 1 to 170 of PD1 (SEQ ID NO: 2) fused to the full length DAP10 (AA 19-93) (SEQ ID NO: 6).

21. The method of claim 17 wherein the chimeric switch receptor comprises amino acids 1 to 170 of PD1 (SEQ ID NO: 2) fused to the full length DAP12 (AA 22-113) (SEQ ID NO: 7) protein.

22. The method of claim 17 wherein the chimeric switch receptor comprises amino acids 1 to 212 of PD1 (SEQ ID NO: 11) fused together with amino acids 77-93 of DAP10 (SEQ ID NO: 12).

23. The method of claim 17 wherein the chimeric switch receptor comprises amino acids 1 to 212 of PD1 (SEQ ID NO: 11) fused together with amino acids 73-113 of DAP12 (SEQ ID NO 13).

24. The method of claim 17 wherein the signaling domain is truncated to omit the transmembrane region.

25. The method of claim 17 wherein the chimeric switch receptor comprises amino acids 52-164 of the CD3ζ protein (SEQ ID NO: 5).

26. The method of claim 17 wherein the chimeric switch receptor comprises amino acids 239-304 of the NKp46 protein (SEQ ID NO 7).

27. The method of claim 17 wherein the chimeric switch receptor comprises the DAP 10 construct of SEQ ID NO 14.

28. The method of claim 17 wherein the chimeric switch receptor comprises the DAP 12 construct of SEQ ID NO 15.

29. The method of claim 17 wherein the chimeric switch receptor comprises the DAP 10 construct of SEQ ID NO 16.

30. The method of claim 17 wherein the chimeric switch receptor comprises the DAP 12 construct of SEQ ID NO 17.

31. A genetic engineering construct comprising SEQ ID NO 5, SEQ ID NO 7, SEQ ID NO 14, SEQ ID NO 15, SEQ ID NO 16, or SEQ ID NO 17.

32. A nucleic acid encoding an NK cell specific PD1-based chimeric switch receptor.

33. A cell comprising a nucleic acid encoding an exogenous chimeric switch receptor.

34. The cell of claim 33, wherein the chimeric switch receptor comprises at least one signaling domain from DAP10, DAP12, NKp46 or CD3ζ.
